(19) United States
(12) Patent Application Publication
(10) Pub. No.: US 2024/0343747 A1
(43) Pub. Date: Oct. 17, 2024

(54) Heteroaryl compounds as inhibitors of TYK2, composition and application thereof

(71) Applicant: Accro Bioscience (HK) Limited, Wan Chai (HK)

(72) Inventors: Xiaohu Zhang, Suzhou (CN); Sudan HE, Suzhou (CN); Haikuo Ma, Suzhou (CN)

(21) Appl. No.: 18/568,840

(22) PCT Filed: Jun. 11, 2022
(86) PCT No.: PCT/US2022/033159

§ 371 Date: Dec. 10, 2023

(30) Foreign Application Priority Data
Jun. 11, 2021 (CN) 202110653751.3
Feb. 28, 2022 (CN) 202210187387.0

(51) Int. Cl. C07F 9/6558; A61K 31/675; A61K 45/06; A61P 17/06; C07F 9/58

(52) U.S. Cl. CPC C07F 9/65583; A61K 31/675; A61K 45/06; A61P 17/06; C07F 9/58; C07F 9/65586

(57) ABSTRACT

The present disclosure provides phosphonate-containing heterocycle compounds with TYK2 kinase inhibitory activity, pharmaceutical compositions comprising the same, and applications thereof. The present disclosure provides compounds of Formula (I), as inhibitors of TYK2 kinase. These compounds can be used for preventing and/or treating TYK2 kinase-related diseases and/or conditions.

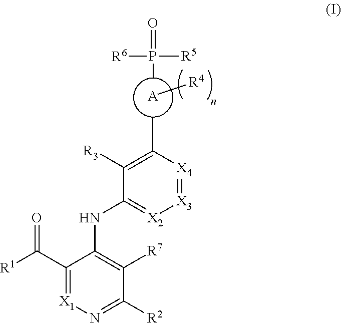

### CROSS REFERENCE TO RELATED APPLICATIONS

This application claims the benefit of Chinese Patent Applications 202110653751.3, filed on Jun. 11, 2021; and, 202210187387.0, filed on Feb. 28, 2022; all of which are hereby incorporated by reference in their entirety.

### FIELD OF INVENTION

The present invention is in the medical technology field, and relates to a compound with TYK2 inhibitory activity. The present invention also relates to compositions comprising the disclosed compounds, their method of making, and their applications in therapies targeting the prevention and/or treatment of diseases associated with TYK2, such as tumors, autoimmune diseases, neurodegenerative diseases, metabolic diseases, and genetic diseases.

### BACKGROUND OF THE INVENTION

Janus kinases or JAKs are non-receptor tyrosine kinases that bind to the intracellular portion of cell surface cytokine receptors. Currently there are four known human JAK family members: JAK1, JAK2, JAK3 and TYK2 (tyrosine kinases 2), each of which contains a kinase domain and a pseudokinase domain (Trends Pharmacol. Sci. 32 (2011), 25-34). JAK1, JAK2 and TYK2 are expressed in various human tissues, while JAK3 is mainly expressed in various hematopoietic cells. A common feature of the cell surface cytokine receptors is that the receptor itself does not have kinase activity, but the intracellular segment of the receptor has a binding site for the tyrosine kinases JAKs. Accordingly, binding of cytokines to receptors results in JAK activation and phosphorylation of JAKs and related receptors. Phosphorylation of the receptors in turn initiates the recruitment of STATs through their SH2 domains and subsequently the phosphorylation of signal transducers and activators of transcription (STAT) proteins. Phosphorylated STAT homodimers or heterodimers then translocate to the nucleus and bind to specific deoxyribonucleic acid (DNA) binding sites to regulate gene transcription, resulting in changes in cellular function (J. Med. Chem., 62 (2019), 8953-8972).

Different paired JAK family members are responsible for signal transduction between different cytokines and their respective receptors. For example, TYK2 modulates interleukin-12 (IL12) and IL23 mediated signal transductions when paired with JAK2; but modulates interferon alpha (IFN-α) mediated signal transduction when paired with JAK1. Since the JAK/STAT pathway is involved in the inflammatory response, it can be a target for the treatment of diseases related to immune disorders (J. Med. Chem., 57 (2014), 5023-5038). As a potential target for treatment of autoimmune disease, TYK2, in particular, garnered support in the research field. For example, mice deficient in TYK2 can survive and develop normally. But deficiency of JAK1 (Cell, 93 (1998), 373-383) or JAK2 (Cell, 93 (1998), 397-409) in mice is lethal. Further, JAK3-deficient mice exhibit severe B- and T-cell depletion (Science, 270 (1995), 800-802). In addition, TYK2 has been shown to be protective in multiple autoimmune deficiency disease models (multiple sclerosis, Crohn's disease, ulcerative colitis, ankylosing spondylitis, and psoriasis, among others) (Brain, 134 (2011), 693-703; Inflammation (London, U. K.) 7 (2010), 41; Nat. Rev. Rheumatol. 12 (2016), 25-36). TYK2 is also associated with some cancers, e.g., T-lineage acute lymphoblastic leukemia (Cancer Disc. 3 (2013), 564-567).

The value of inhibiting pathways involving TYK2 in the treatment of autoimmune diseases has been clinically demonstrated by a variety of antibodies. The antibody Ustekinumab targeting the p40 subunit of both IL-12 and IL-23 is currently marketed for the treatment of psoriasis, psoriatic arthritis and Crohn's disease (Drugs, 71 (2011), 1733-1753; N. Engl. J. Med., 375 (2016), 1946-1960). This antibody recently showed efficacy in patients with systemic lupus erythematosus (SLE) (Lancet, 392 (2018), 1330-1339). The antibody Guselkumab that blocks IL-23 but not IL-12 signaling by targeting the p19 subunit of IL-23 has also been shown to be an effective treatment for psoriasis (J. Am. Acad. Dermatol., 76 (2017), 405-417). Several studies have shown that type 1 interferon has a pathogenic role in systemic lupus erythematosus (SLE), which led to the success of Sifalimumab and Anifrolumab in phase II clinical trials for the treatment of SLE (Ann. Rheum. Dis., 75 (2016), 1909-1916; Arthritis Rheumatol., 69 (2017), 376-386.).

Given that TYK2 may be a therapeutic target, obtaining highly selective TYK2 inhibitors may have a good therapeutic effect on the above diseases. Currently, the selective TYK2 inhibitor BMS-986165 (J. Med. Chem., 62 (2019), 8973-8995) and the JAK1/TYK2 dual inhibitor PF-06700841 (J. Med. Chem., 61 (2018), 8597-8612) have entered clinical trials. Accordingly, obtaining highly druggable, highly active, and highly selective TYK2 or TYK2/JAK1 inhibitors may have promising clinical applications.

### SUMMARY OF THE INVENTION

The present disclosure provides phosphonate-containing heterocycles as selective TYK2 or TYK2/JAK1 inhibitors, and compositions and applications thereof. These disclosed phosphonate-containing heterocycles, and compositions and applications thereof, may effectively prevent or treat diseases and disorders responsive to TYK2 inhibition, including, for example, autoimmune or inflammatory diseases, cancer/tumor, allergy, transplant rejection, neurodegenerative diseases, asthma and other obstructive airway diseases, etc.

One goal of the present disclosure is to provide TYK2 inhibitors, and compositions and applications thereof

An aspect of the present disclosure provides a compound of Formula (I):

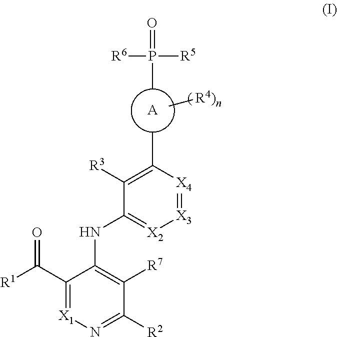

or a pharmaceutically acceptable salt, ester, solvate, prodrug, isotope-labeled derivative, or isomer thereof, wherein:
n is 0, 1, 2 or 3;
X₁ is N or CH;
each of X₂, X₃ and X₄ is independently N or CR⁸;
ring A is C₆₋₁₀ aryl or 5-10 membered heteroaryl;
R¹ is C₁₋₆ alkyl, C₁₋₆ deuterated alkyl, C₂₋₆ alkenyl, C₂₋₆ alkynyl, C₃₋₆ cycloalkyl, —NH(deuterated C₁₋₆ alkyl), or —NH(C₁₋₆ alkyl), wherein C₁₋₆ alkyl, C₁₋₆ deuterated alkyl, C₂₋₆ alkenyl, C₂₋₆ alkynyl, and C₃₋₆ cycloalkyl) are unsubstituted or substituted with one or more groups independently selected from R^{aa}; preferably, R¹ is independently C₁₋₆ alkyl, C₁₋₆ deuterated alkyl, C₂₋₆ alkenyl, C₂₋₆ alkynyl, or C₃₋₆ cycloalkyl, wherein C₁₋₆ alkyl, C₁₋₆ deuterated alkyl, C₂₋₆ alkenyl, C₂₋₆ alkynyl, or C₃₋₆ cycloalkyl, are unsubstituted or substituted with one or more groups independently selected from R^{aa}; R^{aa} is hydrogen, deuterium, halide, amino, —NO₂, —CN, —OH, or C₁₋₃ alkyl;
R² is alkyl, deuterated alkyl, halo-alkyl, alkoxy, halo-alkoxy, alkenyl, alkynyl, cycloalkyl, heterocycloalkyl, aryl, heteroaryl, —NR^{b}R^{c}, —C(O)R^{a}, —C(O)NR^{b}R^{c}, —S(O)R^{a}, —S(O)₂R^{a}, —C(O)OR^{a}, —NR^{d}C(O)R^{a}, —NR^{d}C(O)NR^{b}R^{c}, —NR^{d}S(O)R^{a}, —NR'S(O)₂R^{a}, —NR^{d}S(O)NR^{b}R^{c}, —NR^{d}S(O)₂NR^{b}R^{c}, or —NR^{d}C(O)OR₂, wherein alkyl, deuterated alkyl, halo-alkyl, alkoxy, halo-alkoxy, alkenyl, alkynyl, cycloalkyl, heterocycloalkyl, aryl, and heteroaryl are unsubstituted or substituted with one or more groups independently selected from hydrogen, deuterium, halide, amino, —NO₂, —CN, —OH, C₁₋₃ alkyl, deuterated C₁₋₃ alkyl, C₁₋₃ halo-alkyl, C₁₋₃ alkoxy, deuterated C₁₋₃ alkoxy, C₁₋₃ halo-alkoxy, C₂₋₆ alkenyl, C₂₋₆ alkynyl, substituted or unsubstituted cycloalkyl, substituted and unsubstituted heterocycloalkyl, and substituted or unsubstituted aryl;
R³ is hydrogen, halide, —OH, amino, —SH, —NO₂, —CN, C₁₋₆ alkyl, —C(O)NH₂, C₁₋₆ deuterated alkyl, —O(C₁₋₆ alkyl), —O(C₁₋₆ deuterated alkyl), C₂₋₆ alkenyl, C₂₋₆ alkynyl, C₃₋₆ cycloalkyl, C₃₋₆ heterocycloalkyl, C₆₋₁₀ aryl or 5-10 membered heteroaryl, wherein C₁₋₆ alkyl, C₁₋₆ deuterated alkyl, —O(C₁₋₆ alkyl), —O(C₁₋₆ deuterated alkyl), C₂₋₆ alkenyl, C₂₋₆ alkynyl, C₃₋₆ cycloalkyl, C₃₋₆ heterocycloalkyl, C₆₋₁₀ aryl and 5-10 membered heteroaryl are unsubstituted or substituted with one or more groups independently selected from R^{aa};
if present, each R⁴ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R^{bb} is hydrogen, deuterium, halide, amino, —NO₂, —CN, and —OH;
each of R⁵ and R⁶ is independently C₁₋₆ alkyl, C₁₋₆ deuterated alkyl, C₂₋₆ alkenyl, C₂₋₆ alkynyl, or C₃₋₆ cycloalkyl, wherein C₁₋₆ alkyl, C₁₋₆ deuterated alkyl, C₂₋₆ alkenyl, C₂₋₆ alkynyl, and C₃₋₆ cycloalkyl are unsubstituted or substituted with one or more groups selected from R^{bb}; preferably each of R⁵ and R⁶ is independently C₁₋₃ alkyl, wherein C₁₋₃ alkyl is unsubstituted or substituted with one or more groups selected from R^{bb}; or R⁵ and R⁶, together with P attached thereto, form a 5-6 membered heterocycloalkyl, wherein 5-6 membered heterocycloalkyl is unsubstituted or substituted with one or more groups selected from R^{bb};
R⁷ is hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
if present, each R⁸ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb}; and
each of R^{a}, R^{b}, R^{c} and R^{d} is independently hydrogen, deuterium, halide, amino, —NO₂, —CN, —OH, alkyl, deuterated alkyl, halo-alkyl, alkoxy, halo-alkoxy, alkenyl, alkynyl, cycloalkyl, heterocycloalkyl, aryl, or heteroaryl, wherein alkyl, deuterated alkyl, halo-alkyl, alkoxy, halo-alkoxy, alkenyl, alkynyl, cycloalkyl, heterocycloalkyl, aryl, and heteroaryl are unsubstituted or substituted with one or more groups independently selected from hydrogen, deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted cycloalkyl, and substituted and unsubstituted heterocycloalkyl, substituted or unsubstituted aryl, and substituted or unsubstituted heteroaryl; or any two of adjacent or non-adjacent R^{a}, R^{b}, R^{c} and R^{d} form a cycloalkyl, heterocycloalkyl, aryl or heteroaryl, wherein cycloalkyl, heterocycloalkyl, aryl and heteroaryl are unsubstituted or substituted with one or more groups selected from hydrogen, deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted 6-10 membered aryl, and substituted or unsubstituted 5-10 membered heteroaryl.

In some embodiments of aspects provided herein, including any one of the hitherto described embodiments, R² is C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, 5-10 membered heteroaryl, —NR^{b}R^{c}, —C(O)R^{a}, —C(O)NR^{b}R^{c}, —S(O)R^{a}, —S(O)₂R^{a}, —C(O)OR^{a}, —NR^{d}C(O)R^{a}, —NR^{d}C(O)NR^{b}R^{c}, —NR^{d}S(O)R^{a}, —NR^{d}S(O)₂R^{a}, —NR^{d}S(O)NR^{b}R^{c}, —NR^{d}S(O)₂NR^{b}R^{c}, or —NR^{d}C(O)OR₂, wherein C₁₋₆ alkyl, deuterated C₁₋₆ alkyl, C₁₋₆ halo-alkyl, C₁₋₆ alkoxy, C₁₋₆ halo-alkoxy, C₂₋₆ alkynyl, C₃₋₆ cycloalkyl, 3-6 membered heterocycloalkyl, C₆₋₁₀ aryl, and 5-10 membered heteroaryl are unsubstituted or substituted with one or more groups independently selected from hydrogen, deuterium, halide, amino, —NO₂, —CN, —OH, C₁₋₃ alkyl, deuterated C₁₋₃ alkyl, C₁₋₃ halo-alkyl, C₁₋₃ alkoxy, deuterated C₁₋₃ alkoxy, C₁₋₃ halo-alkoxy, C₂₋₃ alkenyl, C₂₋₃ alkynyl, substituted or unsubstituted C₃₋₆ cycloalkyl, substituted and unsubstituted 3-6 membered heterocycloalkyl, substituted or unsubstituted C₆₋₁₀ aryl, and substituted or unsubstituted 5-10 membered heteroaryl; and
each of R^{a}, R^{b}, R^{c} and R^{d} is independently hydrogen, deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl, wherein C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, and 5-10 membered heteroaryl are unsubstituted or substituted with one or more groups independently selected from hydrogen, deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, and substituted and unsubstituted 3-6 membered heterocycloalkyl, substituted or unsubstituted C₆₋₁₀ aryl, and substituted or unsubstituted 5-10 membered heteroaryl; or any two of adjacent or non-adjacent R^{a}, R^{b}, R^{c} and R^{d} form a C₃₋₆ cycloalkyl, C₃₋₆ heterocycloalkyl, C₆₋₁₀ aryl or 5-10 membered heteroaryl, wherein C₃₋₆ cycloalkyl, C₃₋₆ heterocycloalkyl, C₆₋₁₀ aryl and 5-10 membered heteroaryl are unsubstituted or substituted with one or more groups selected from hydrogen, deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted C₆₋₁₀ aryl, and substituted or unsubstituted 5-10 membered heteroaryl.

In some embodiments, including any one of the hitherto described embodiments, ring A is phenyl or 5-6 membered heteroaryl;
R¹ is C₁-3 alkyl, C₁₋₃ deuterated alkyl, C₂₋₆ alkenyl, C₂₋₆ alkynyl, C₃₋₆ cycloalkyl, —NH(deuterated C₁₋₃ alkyl), or —NH(C₁₋₆ alkyl), wherein C₁₋₃ alkyl, C₁₋₃ deuterated alkyl, C₂₋₆ alkenyl, C₂₋₆ alkynyl, and C₃₋₆ cycloalkyl are unsubstituted or substituted with one or more groups independently selected from R^{aa}; preferably, R¹ is independently C₁₋₃ alkyl, C₁₋₃ deuterated alkyl, C₂₋₆ alkenyl, C₂₋₆ alkynyl, or C₃₋₆ cycloalkyl;
R³ is-CN, C₁₋₆ alkyl, C₁₋₆ deuterated alkyl, —O(C₁₋₆ alkyl), —O(C₁₋₆ deuterated alkyl), C₃₋₆ cycloalkyl, or —C(O)NH₂;
R⁵ is C₁₋₆ alkyl or C₃₋₆ cycloalkyl; preferably C₁₋₃ alkyl or cyclopropyl; and
R⁶ is C₁₋₆ alkyl or C₃₋₆ cycloalkyl; preferably C₁₋₃ alkyl or cyclopropyl.

In some embodiments, the compound is of Formula (II):

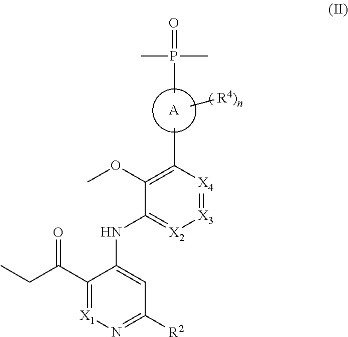

or a pharmaceutically acceptable salt, ester, solvate, prodrug, isotope-labeled derivative, or isomer thereof, wherein:
n is 0, 1, 2 or 3;
X₁ is N or CH;
each of X₂, X₃ and X₄ is independently N or CR⁸;
ring A is C₆₋₁₀ aryl or 5-10 membered heteroaryl;
R² is alkyl, deuterated alkyl, halo-alkyl, alkoxy, halo-alkoxy, alkenyl, alkynyl, cycloalkyl, heterocycloalkyl, aryl, heteroaryl, —NR^{b}R^{c}, —C(O)R^{a}, —C(O)NR^{b}R^{c}, —S(O)R^{a}, —S(O)₂R^{a}, —C(O)OR^{a}, —NR^{d}C(O)R^{a}, —NR^{d}C(O)NR^{b}R^{c}, —NR^{d}S(O)R^{a}, —NR^{d}S(O)₂R^{a}, —NR^{d}S(O)NR^{b}R^{c}, —NR^{d}S(O)₂NR^{b}R^{c}, or —NR^{d}C(O)OR₂, wherein alkyl, deuterated alkyl, halo-alkyl, alkoxy, halo-alkoxy, alkenyl, alkynyl, cycloalkyl, heterocycloalkyl, aryl, and heteroaryl are unsubstituted or substituted with one or more groups independently selected from hydrogen, deuterium, halide, amino, —NO₂, —CN, —OH, C₁₋₃ alkyl, deuterated C₁₋₃ alkyl, C₁₋₃ halo-alkyl, C₁₋₃ alkoxy, deuterated C₁₋₃ alkoxy, C₁₋₃ halo-alkoxy, C₂₋₆ alkenyl, C₂₋₆ alkynyl, substituted or unsubstituted cycloalkyl, substituted and unsubstituted heterocycloalkyl, and substituted or unsubstituted aryl; if present, each R⁴ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R^{bb} is hydrogen, deuterium, halide, amino, —NO₂, —CN, and —OH;
if present, each R⁸ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb}; and
each of R^{a}, R^{b}, R^{c} and R^{d} is independently hydrogen, deuterium, halide, amino, —NO₂, —CN, —OH, alkyl, deuterated alkyl, halo-alkyl, alkoxy, halo-alkoxy, alkenyl, alkynyl, cycloalkyl, heterocycloalkyl, aryl, or heteroaryl, wherein alkyl, deuterated alkyl, halo-alkyl, alkoxy, halo-alkoxy, alkenyl, alkynyl, cycloalkyl, heterocycloalkyl, aryl, and heteroaryl are unsubstituted or substituted with one or more groups independently selected from hydrogen, deuterium, halide, amino, —NO₂, —CN, —OH, C₁₋₃ alkyl, deuterated C₁₋₃ alkyl, C₁₋₃ halo-alkyl, C₁₋₃ alkoxy, C₁₋₃ halo-alkoxy, C₂₋₆ alkenyl, C₂₋₆ alkynyl, substituted or unsubstituted cycloalkyl, and substituted and unsubstituted heterocycloalkyl, substituted or unsubstituted aryl, and substituted or unsubstituted heteroaryl; or any two of adjacent or non-adjacent R^{a}, R^{b}, R^{c} and R^{d} form a cycloalkyl, heterocycloalkyl, aryl or heteroaryl, wherein cycloalkyl, heterocycloalkyl, aryl and heteroaryl are unsubstituted or substituted with one or more groups selected from hydrogen, deuterium, halide, amino, —NO₂, —CN, —OH, C₁₋₃ alkyl, deuterated C₁₋₃ alkyl, C₁₋₃ halo-alkyl, C₁₋₃ alkoxy, C₁₋₃ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted 6-10 membered aryl, and substituted or unsubstituted 5-10 membered heteroaryl.

In some embodiments, including any one of the hitherto described embodiments,

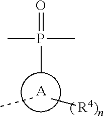

is a moiety A substituted with 0 to 3 R¹⁰,
wherein, if present, each R¹⁰ is independently hydrogen, deuterium, halide, amino, —CN, —OH, C₁₋₃ alkyl or C₁₋₃ alkoxy; and
wherein the moiety A is selected from the group consisting of:

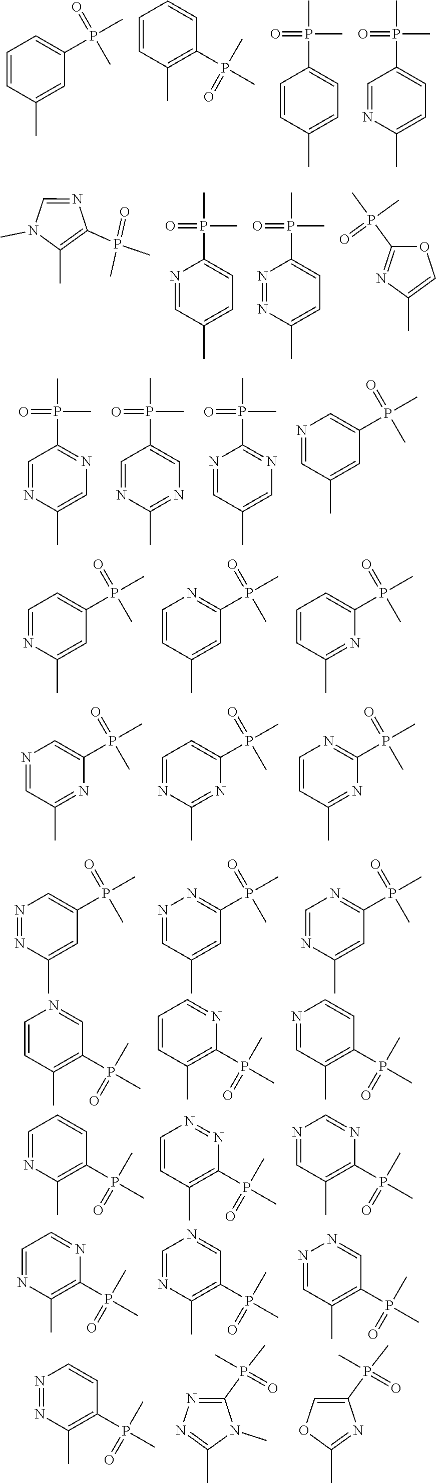

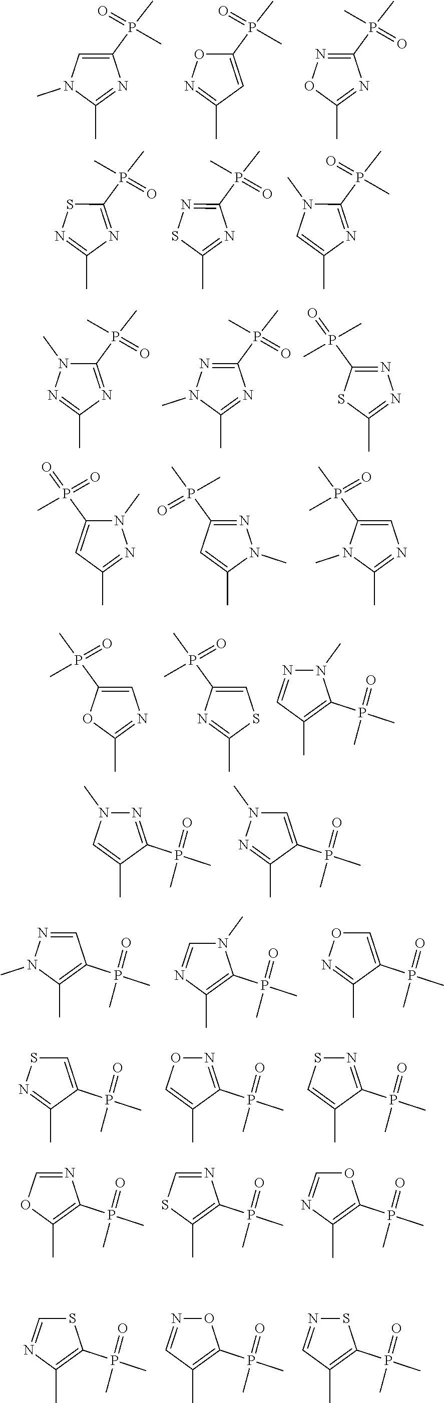

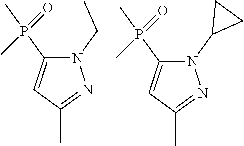

In some embodiments, including any one of the hitherto described embodiments, R² is a moiety B substituted with 0 to 3 R¹¹; wherein, if present, each R¹¹ is independently hydrogen, deuterium, halide, amino, —NO₂, —CN, —OH, C₁₋₃ alkyl or C₁₋₃ alkoxy; and wherein the moiety B is selected from the group consisting of:

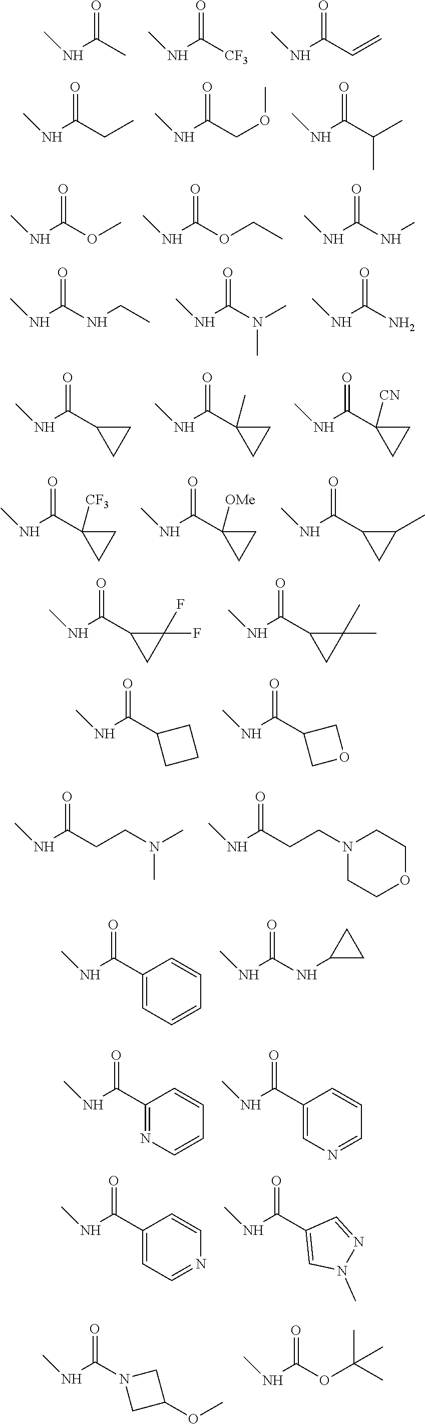

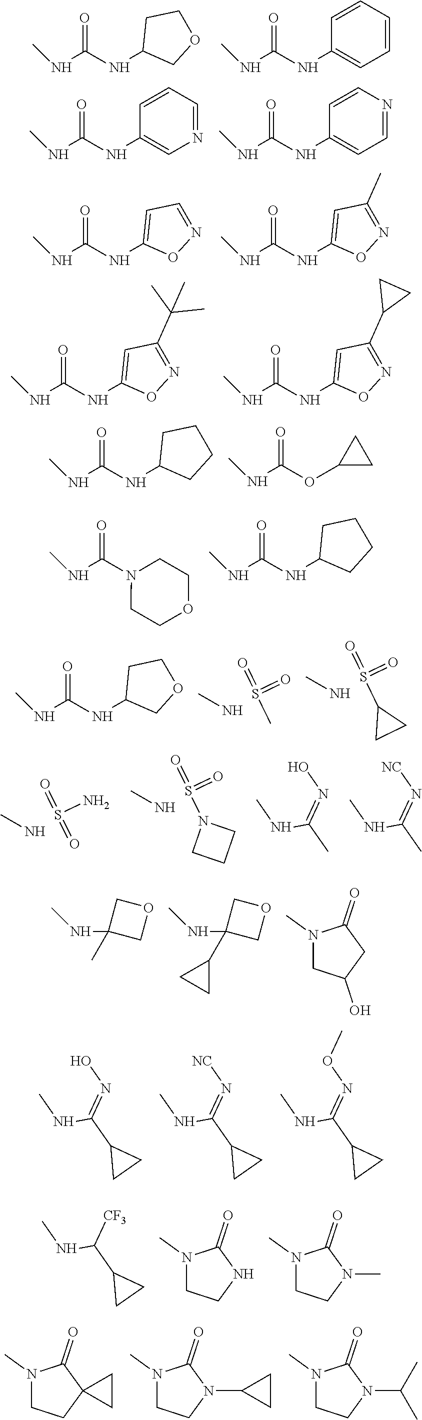

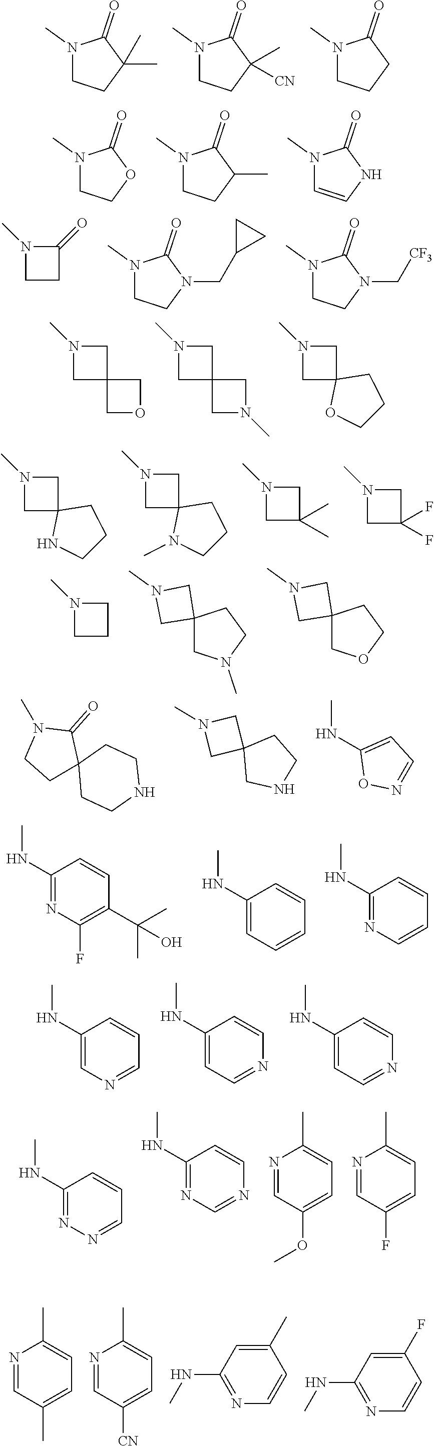

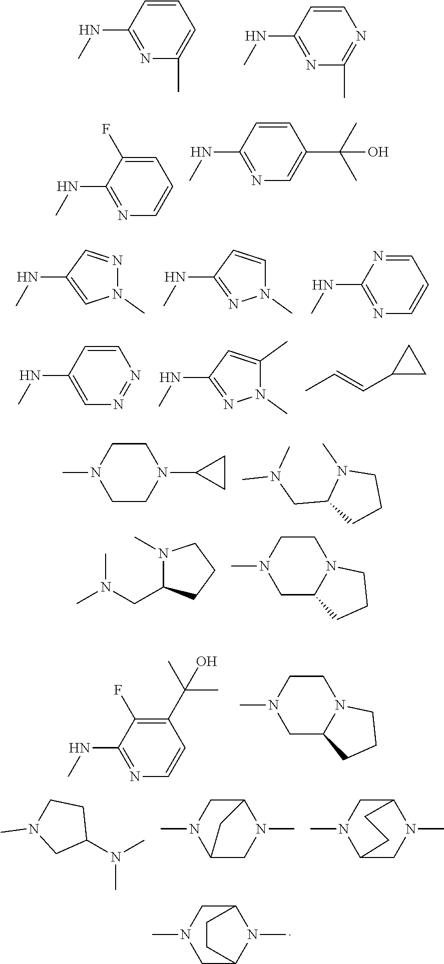

In some embodiments, the compound is of Formula (III):

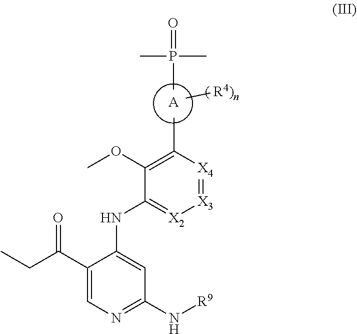

or a pharmaceutically acceptable salt, ester, solvate, prodrug, isotope-labeled derivative, or isomer thereof, wherein:
n is 0, 1, 2 or 3;
each of X₂, X₃ and X₄ is independently N or CR⁸;
ring A is C₆₋₁₀ aryl or 5-10 membered heteroaryl;
if present, each R⁴ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R^{bb} is hydrogen, deuterium, halide, amino, —NO₂, —CN, and —OH;
if present, each R⁸ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R⁹ is C₆₋₁₀ aryl, 5-10 membered heteroaryl, —C(O)R^{a}, —C(O)NR^{b}R^{c}, —S(O)R^{a}, —S(O)₂R³, —S(O)NR^{b}R^{c}, —S(O)₂NR^{b}R^{c}, or —C(O)OR₂, wherein C₆₋₁₀ aryl and 5-10 membered heteroaryl are substituted by one or more groups selected from hydrogen, deuterium, halide, amino, —NO₂, —CN, —OH, C₁₋₃ alkyl, C₁₋₃ deuterated alkyl, C₁₋₃ halo-alkyl C₁₋₃ alkoxy, C₁₋₃ halo-alkoxy, C₂₋₃ alkenyl, and C₂₋₃ alkynyl; and
each of R^{a}, R^{b}, and R^{c} is independently hydrogen, deuterium, halide, amino, —NO₂, —CN, —OH, C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl, wherein C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl are unsubstituted or substituted with one or more groups independently selected from hydrogen, deuterium, halide, amino, —NO₂, —CN, —OH, C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted C₆₋₁₀ aryl, and substituted or unsubstituted 5-10 membered heteroaryl.

In some embodiments, the compound is of Formula (IV):

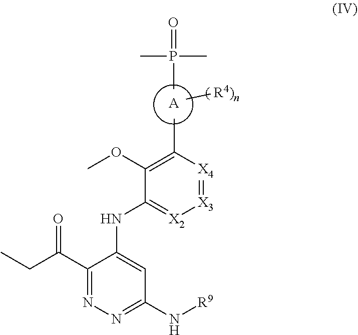

or a pharmaceutically acceptable salt, ester, solvate, prodrug, isotope-labeled derivative, or isomer thereof, wherein:
n is 0, 1, 2 or 3;
each of X₂, X₃ and X₄ is independently N or CR⁸;
ring A is C₆₋₁₀ aryl or 5-10 membered heteroaryl;
if present, each R⁴ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R^{bb} is hydrogen, deuterium, halide, amino, —NO₂, —CN, and —OH;
if present, each R⁸ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R⁹ is C₆₋₁₀ aryl, 5-10 membered heteroaryl, —C(O)R^{a}, —C(O)NR^{b}R^{c}, —S(O)R^{a}, —S(O)₂R^{a}, —S(O)NR^{b}R^{c}, —S(O)₂NR^{b}R^{c}, or —C(O)OR^{a}, wherein C₆₋₁₀ aryl and 5-10 membered heteroaryl are substituted by one or more groups selected from hydrogen, deuterium, halide, amino, —NO₂, —CN, —OH, C₁₋₃ alkyl, C₁₋₃ deuterated alkyl, C₁₋₃ halo-alkyl C₁₋₃ alkoxy, C₁₋₃ halo-alkoxy, C₂₋₃ alkenyl, and C₂₋₃ alkynyl; and
each of R^{a}, R^{b}, and R^{c} is independently hydrogen, deuterium, halide, amino, —NO₂, —CN, —OH, C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl, wherein C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl are unsubstituted or substituted with one or more groups independently selected from hydrogen, deuterium, halide, amino, —NO₂, —CN, —OH, C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted C₆₋₁₀ aryl, and substituted or unsubstituted 5-10 membered heteroaryl.

In some embodiments, including any one of the hitherto described embodiments,

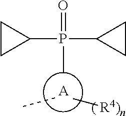

is a moiety C substituted with 0 to 3 R¹⁰,
wherein, if present, each R¹⁰ is independently hydrogen, deuterium, halide, amino, —CN, —OH, C₁₋₃ alkyl or C₁₋₃ alkoxy; and
wherein the moiety C is selected from the group consisting of:

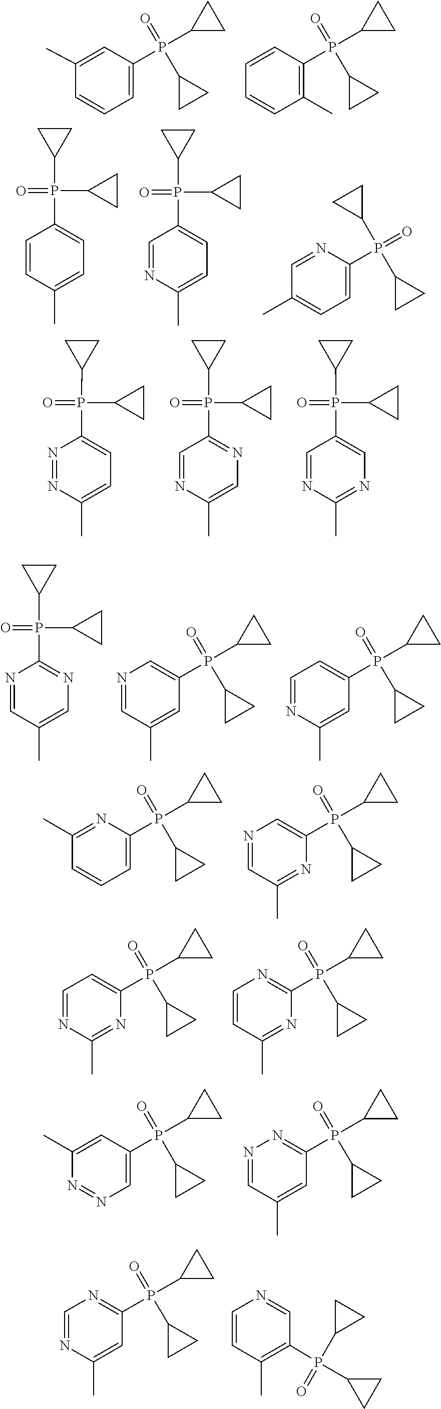

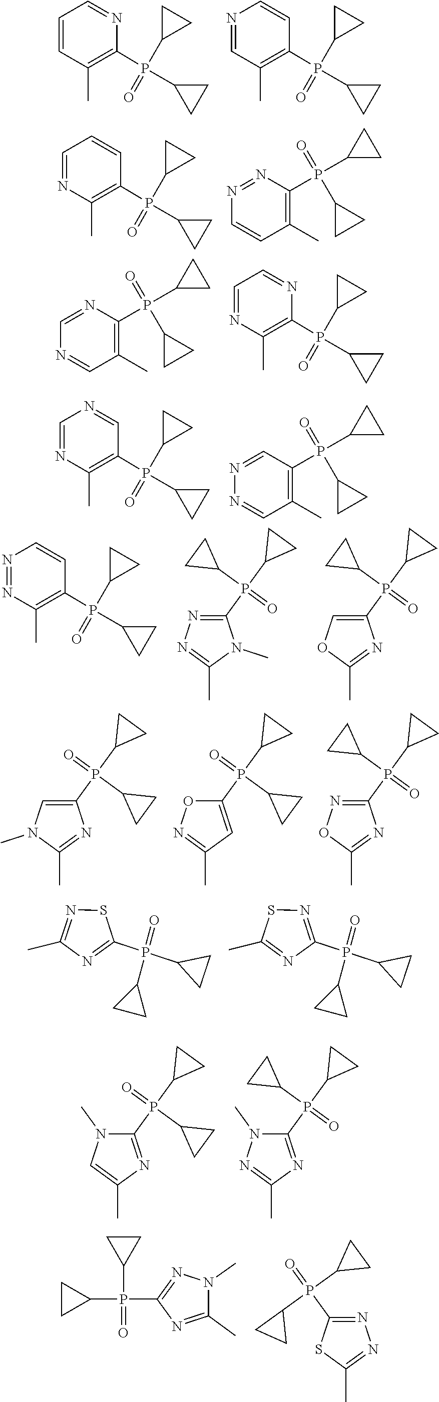

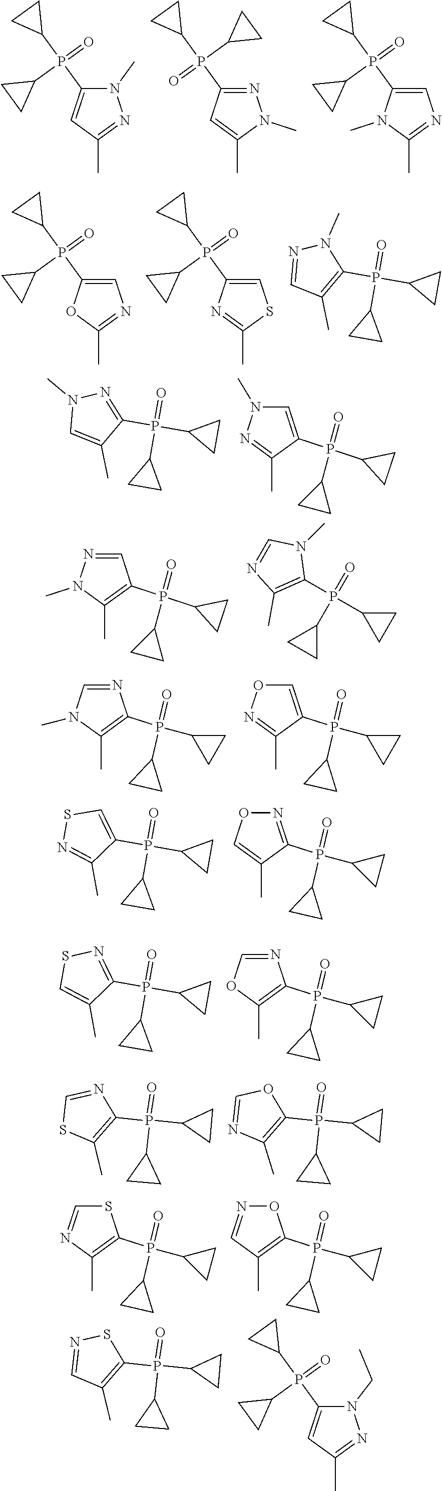

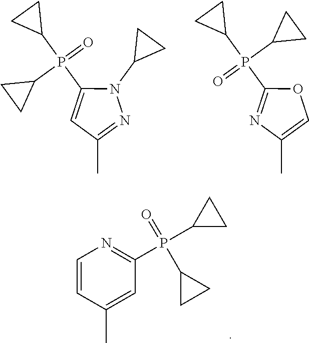

In some embodiments, including any one of the hitherto described embodiments, R² is a moiety D substituted with 0 to 3 R¹¹;
wherein, if present, each R¹¹ is independently hydrogen, deuterium, halide, amino, —NO₂, —CN, —OH, C₁₋₃ alkyl or C₁₋₃ alkoxy; and
wherein the moiety D is selected from the group consisting of:

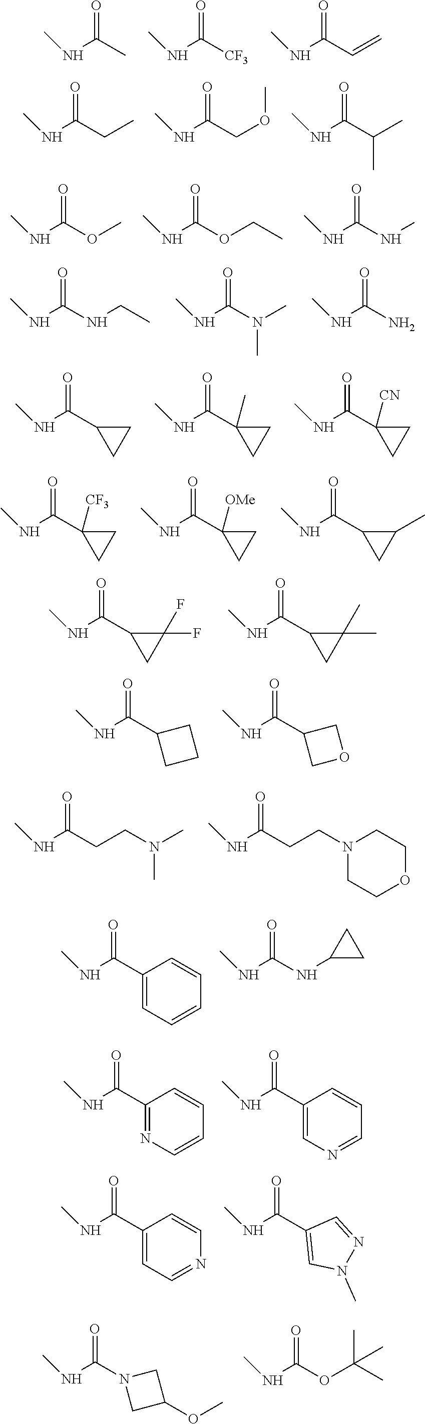

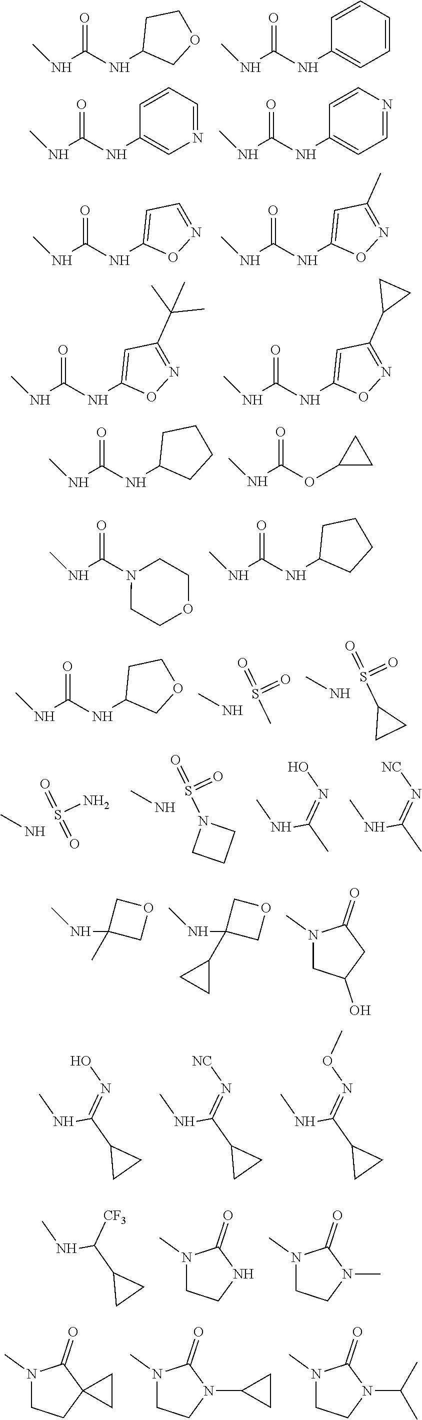

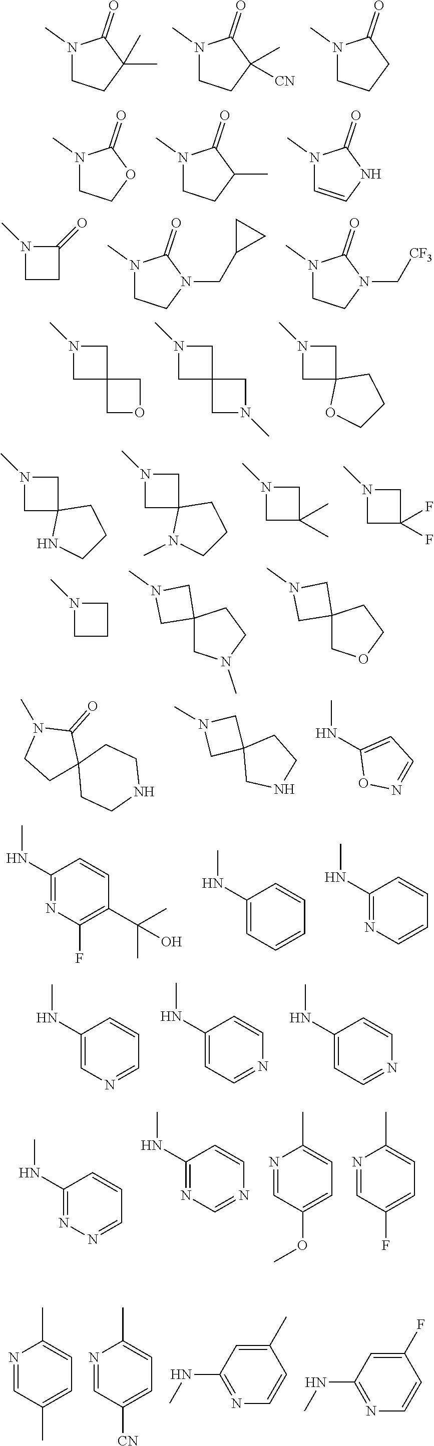

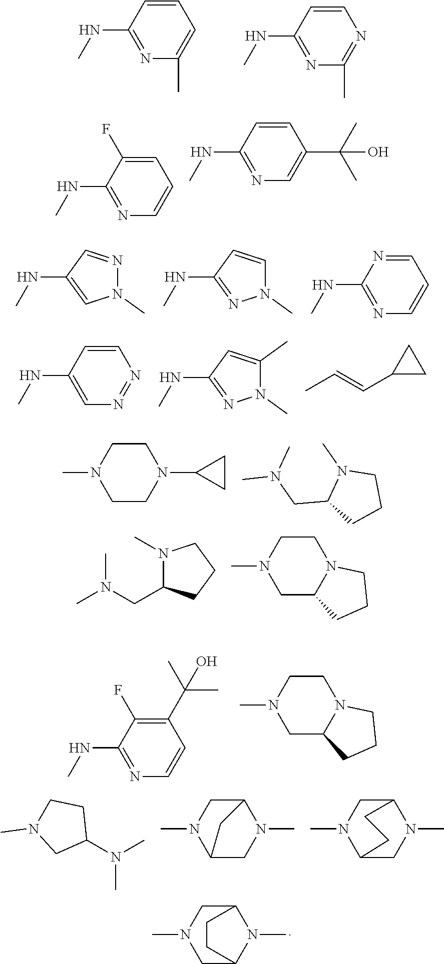

In some embodiments, the compound is of Formula (VI):

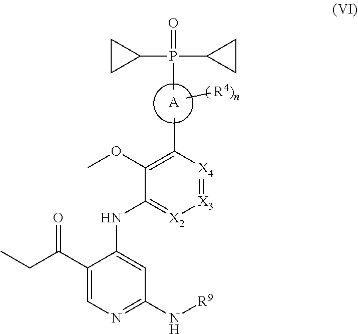

or a pharmaceutically acceptable salt, ester, solvate, prodrug, isotope-labeled derivative, or isomer thereof, wherein:
n is 0, 1, 2 or 3;
each of X₂, X₃ and X₄ is independently N or CR⁸;
ring A is C₆₋₁₀ aryl or 5-10 membered heteroaryl;
if present, each R⁴ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R^{bb} is hydrogen, deuterium, halide, amino, —NO₂, —CN, and —OH;
if present, each R⁸ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R⁹ is C₆₋₁₀ aryl, 5-10 membered heteroaryl, —C(O)R^{a}, —C(O)NR^{b}R^{c}, —S(O)R^{a}, —S(O)₂R^{a}, —S(O)NR^{b}R^{c}, —S(O)₂NR^{b}R^{c}, or —C(O)OR^{a}, wherein C₆₋₁₀ aryl and 5-10 membered heteroaryl are substituted by one or more groups selected from hydrogen, deuterium, halide, amino, —NO₂, —CN, —OH, C₁₋₃ alkyl, C₁₋₃ deuterated alkyl, C₁₋₃ halo-alkyl C₁₋₃ alkoxy, C₁₋₃ halo-alkoxy, C₂₋₃ alkenyl, and C₂₋₃ alkynyl; and
each of R^{a}, R^{b}, and R^{c} is independently hydrogen, deuterium, halide, amino, —NO₂, —CN, —OH, C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl, wherein C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl are unsubstituted or substituted with one or more groups independently selected from hydrogen, deuterium, halide, amino, —NO₂, —CN, —OH, C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted C₆₋₁₀ aryl, and substituted or unsubstituted 5-10 membered heteroaryl.

In some embodiments, the compound is of Formula (VII):

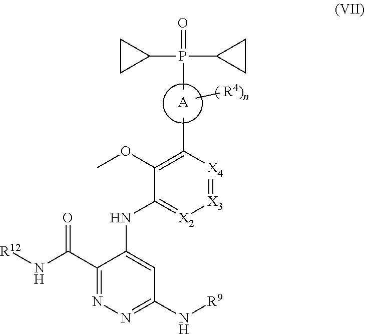

or a pharmaceutically acceptable salt, ester, solvate, prodrug, isotope-labeled derivative, or isomer thereof, wherein:
n is 0, 1, 2 or 3;
each of X₂, X₃ and X₄ is independently N or CR⁸;
ring A is C₆₋₁₀ aryl or 5-10 membered heteroaryl;
if present, each R⁴ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R^{bb} is hydrogen, deuterium, halide, amino, —NO₂, —CN, and —OH;
if present, each R⁸ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R⁹ is C₆₋₁₀ aryl, 5-10 membered heteroaryl, —C(O)R^{a}, —C(O)NR^{b}R^{c}, —S(O)R^{a}, —S(O)₂R³, —S(O)NR^{b}R^{c}, —S(O)₂NR^{b}R^{c}, or —C(O)OR^{a}, wherein C₆₋₁₀ aryl and 5-10 membered heteroaryl are substituted by one or more groups selected from hydrogen, deuterium, halide, amino, —NO₂, —CN, —OH, C₁₋₃ alkyl, C₁₋₃ deuterated alkyl, C₁₋₃ halo-alkyl C₁₋₃ alkoxy, C₁₋₃ halo-alkoxy, C₂₋₃ alkenyl, and C₂₋₃ alkynyl;
R¹² is methyl, —CH₂D, —CHD₂, or —CD₃; and
each of R^{a}, R^{b}, and R^{c} is independently hydrogen, deuterium, halide, amino, —NO₂, —CN, —OH, C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl, wherein C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl are unsubstituted or substituted with one or more groups independently selected from hydrogen, deuterium, halide, amino, —NO₂, —CN, —OH, C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted C₆₋₁₀ aryl, and substituted or unsubstituted 5-10 membered heteroaryl.

Another aspect of the present disclosure provides a compound or a pharmaceutically acceptable salt, ester, solvate, prodrug, isotope-labeled derivative, or isomer thereof, wherein the compound is selected from the group consisting of:

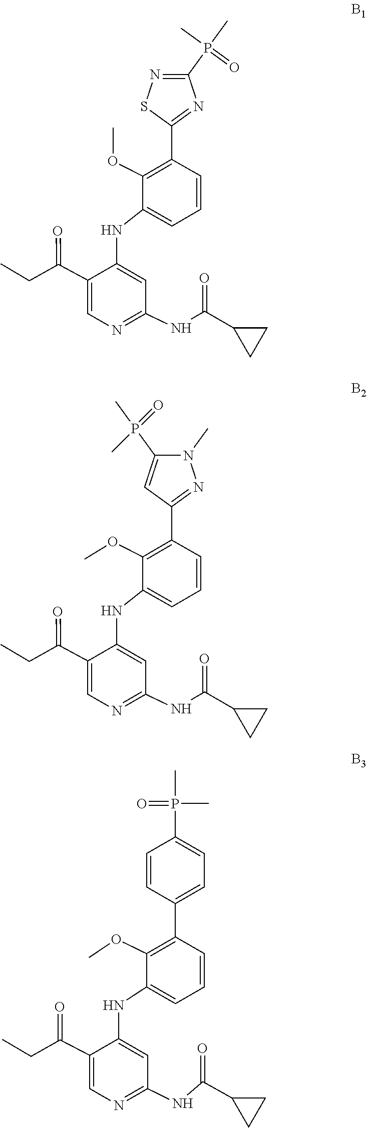 [SMILES: CCC(=O)c1cnc(NC(=O)C2CC2)cc1Nc1cccc(-c2nc(P(C)(C)=O)ns2)c1OC ; CCC(=O)c1cnc(NC(=O)C2CC2)cc1Nc1cccc(-c2cc(P(C)(C)=O)n(C)n2)c1OC ; CCC(=O)c1cnc(NC(=O)C2CC2)cc1Nc1cccc(-c2ccc(P(C)(C)=O)cc2)c1OC]

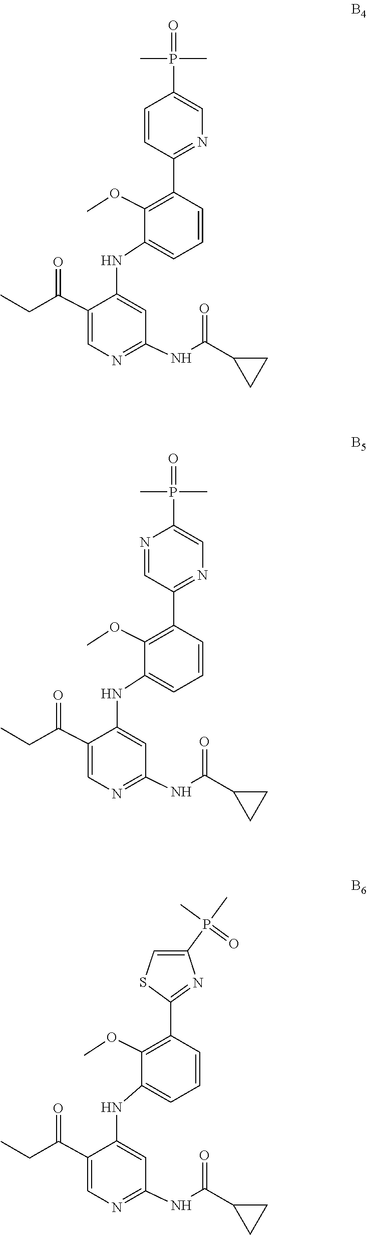 [SMILES: CCC(=O)c1cnc(NC(=O)C2CC2)cc1Nc1cccc(-c2ccc(P(C)(C)=O)cn2)c1OC ; CCC(=O)c1cnc(NC(=O)C2CC2)cc1Nc1cccc(-c2cnc(P(C)(C)=O)cn2)c1OC ; CCC(=O)c1cnc(NC(=O)C2CC2)cc1Nc1cccc(-c2nc(P(C)(C)=O)cs2)c1OC]

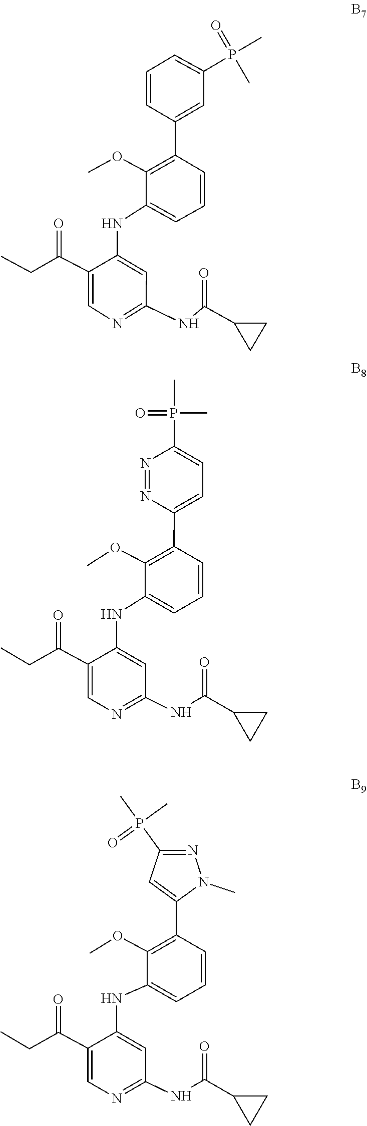 [SMILES: CCC(=O)c1cnc(NC(=O)C2CC2)cc1Nc1cccc(-c2cccc(P(C)(C)=O)c2)c1OC ; CCC(=O)c1cnc(NC(=O)C2CC2)cc1Nc1cccc(-c2ccc(P(C)(C)=O)nn2)c1OC ; CCC(=O)c1cnc(NC(=O)C2CC2)cc1Nc1cccc(-c2cc(P(C)(C)=O)nn2C)c1OC]

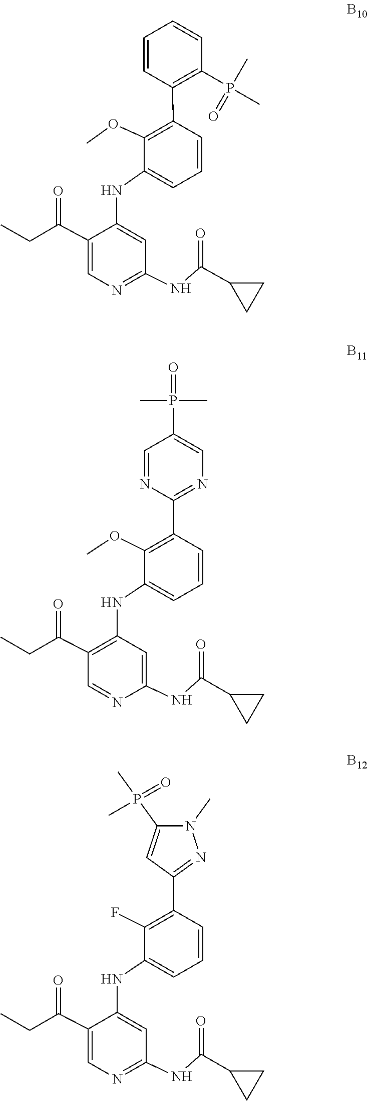 [SMILES: CCC(=O)c1cnc(NC(=O)C2CC2)cc1Nc1cccc(-c2ccccc2P(C)(C)=O)c1OC ; CCC(=O)c1cnc(NC(=O)C2CC2)cc1Nc1cccc(-c2ncc(P(C)(C)=O)cn2)c1OC ; CCC(=O)c1cnc(NC(=O)C2CC2)cc1Nc1cccc(-c2cc(P(C)(C)=O)n(C)n2)c1F]

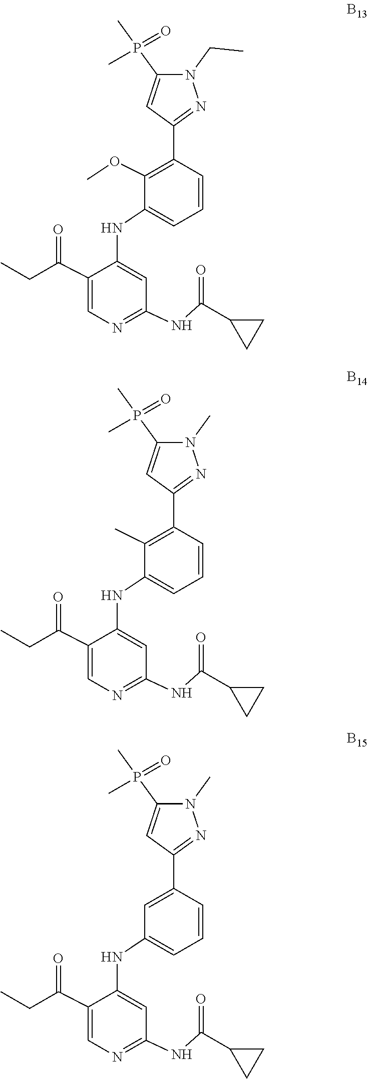 [SMILES: CCC(=O)c1cnc(NC(=O)C2CC2)cc1Nc1cccc(-c2cc(P(C)(C)=O)n(CC)n2)c1OC ; CCC(=O)c1cnc(NC(=O)C2CC2)cc1Nc1cccc(-c2cc(P(C)(C)=O)n(C)n2)c1C ; CCC(=O)c1cnc(NC(=O)C2CC2)cc1Nc1cccc(-c2cc(P(C)(C)=O)n(C)n2)c1]

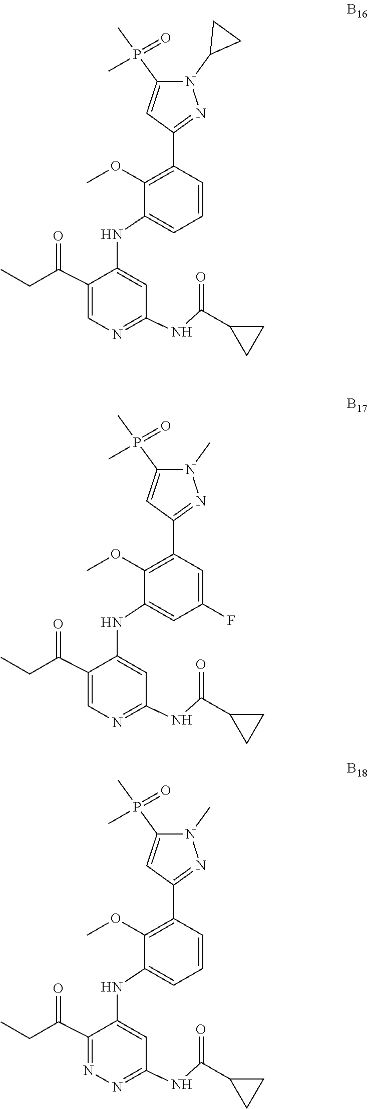 [SMILES: CCC(=O)c1cnc(NC(=O)C2CC2)cc1Nc1cccc(-c2cc(P(C)(C)=O)n(C3CC3)n2)c1OC ; CCC(=O)c1cnc(NC(=O)C2CC2)cc1Nc1cc(F)cc(-c2cc(P(C)(C)=O)n(C)n2)c1OC ; CCC(=O)c1nnc(NC(=O)C2CC2)cc1Nc1cccc(-c2cc(P(C)(C)=O)n(C)n2)c1OC]

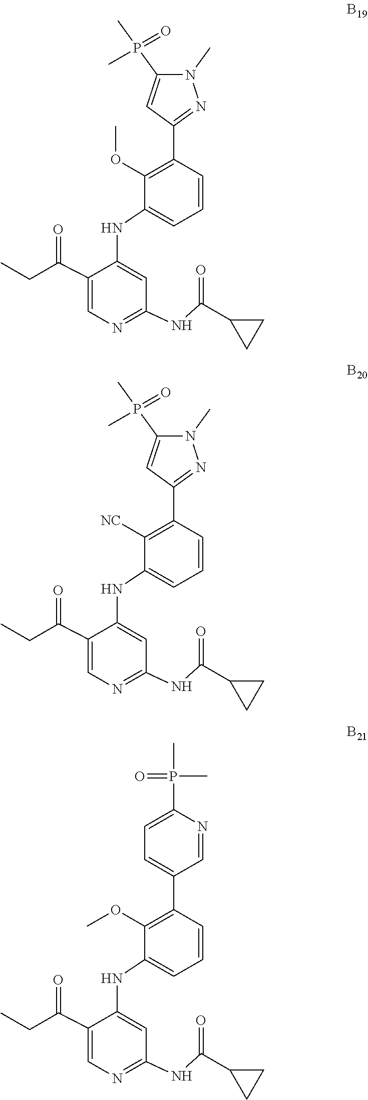 [SMILES: CCC(=O)c1cnc(NC(=O)C2CC2)cc1Nc1cccc(-c2cc(P(C)(C)=O)n(C)n2)c1OC ; CCC(=O)c1cnc(NC(=O)C2CC2)cc1Nc1cccc(-c2cc(P(C)(C)=O)n(C)n2)c1C#N ; CCC(=O)c1cnc(NC(=O)C2CC2)cc1Nc1cccc(-c2ccc(P(C)(C)=O)nc2)c1OC]

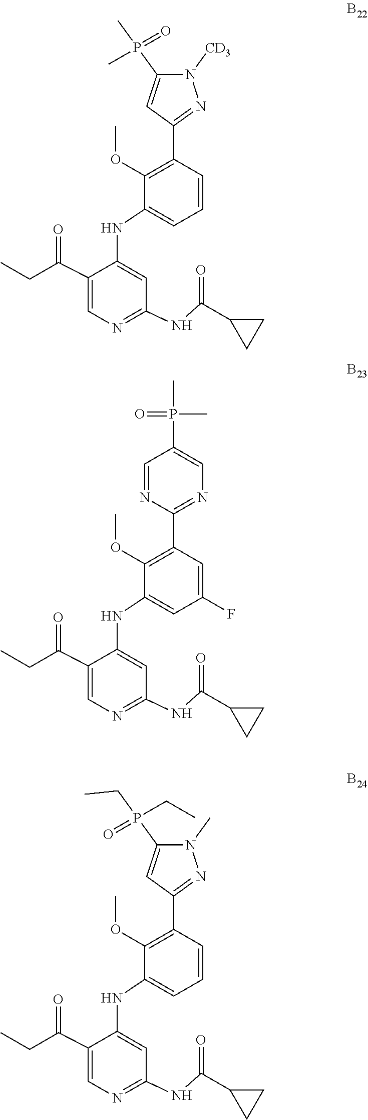 [SMILES: [2H]C([2H])([2H])n1nc(-c2cccc(Nc3cc(NC(=O)C4CC4)ncc3C(=O)CC)c2OC)cc1P(C)(C)=O ; CCC(=O)c1cnc(NC(=O)C2CC2)cc1Nc1cc(F)cc(-c2ncc(P(C)(C)=O)cn2)c1OC ; CCC(=O)c1cnc(NC(=O)C2CC2)cc1Nc1cccc(-c2cc(P(=O)(CC)CC)n(C)n2)c1OC]

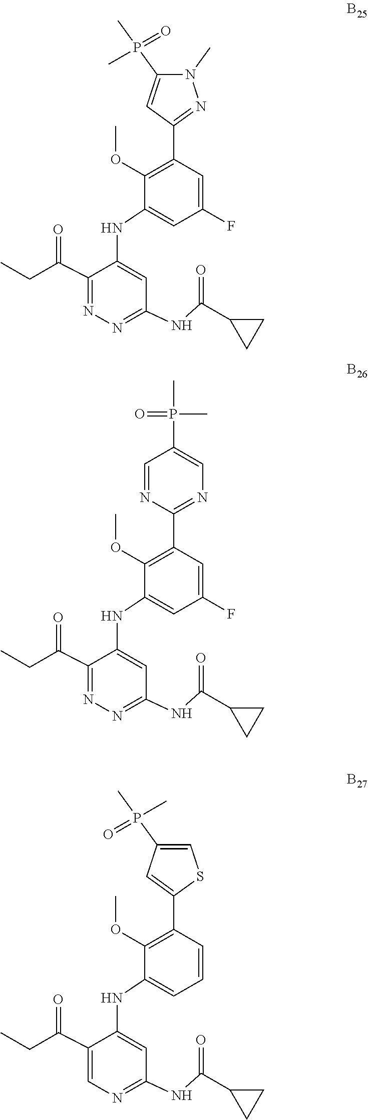 [SMILES: CCC(=O)c1nnc(NC(=O)C2CC2)cc1Nc1cc(F)cc(-c2cc(P(C)(C)=O)n(C)n2)c1OC ; CCC(=O)c1nnc(NC(=O)C2CC2)cc1Nc1cc(F)cc(-c2ncc(P(C)(C)=O)cn2)c1OC ; CCC(=O)c1cnc(NC(=O)C2CC2)cc1Nc1cccc(-c2cc(P(C)(C)=O)cs2)c1OC]

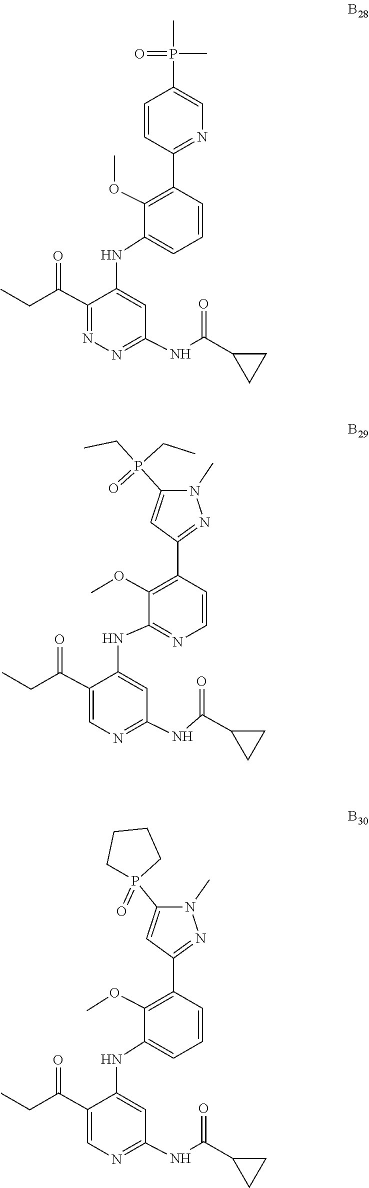 [SMILES: CCC(=O)c1nnc(NC(=O)C2CC2)cc1Nc1cccc(-c2ccc(P(C)(C)=O)cn2)c1OC ; CCC(=O)c1cnc(NC(=O)C2CC2)cc1Nc1nccc(-c2cc(P(=O)(CC)CC)n(C)n2)c1OC ; CCC(=O)c1cnc(NC(=O)C2CC2)cc1Nc1cccc(-c2cc(P3(=O)CCCC3)n(C)n2)c1OC]

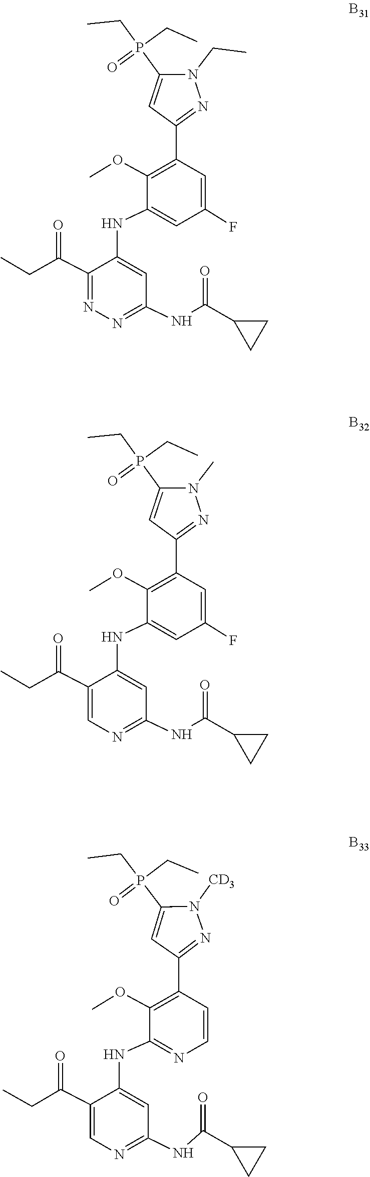 [SMILES: CCC(=O)c1nnc(NC(=O)C2CC2)cc1Nc1cc(F)cc(-c2cc(P(=O)(CC)CC)n(CC)n2)c1OC ; CCC(=O)c1cnc(NC(=O)C2CC2)cc1Nc1cc(F)cc(-c2cc(P(=O)(CC)CC)n(C)n2)c1OC ; [2H]C([2H])([2H])n1nc(-c2ccnc(Nc3cc(NC(=O)C4CC4)ncc3C(=O)CC)c2OC)cc1P(=O)(CC)CC]

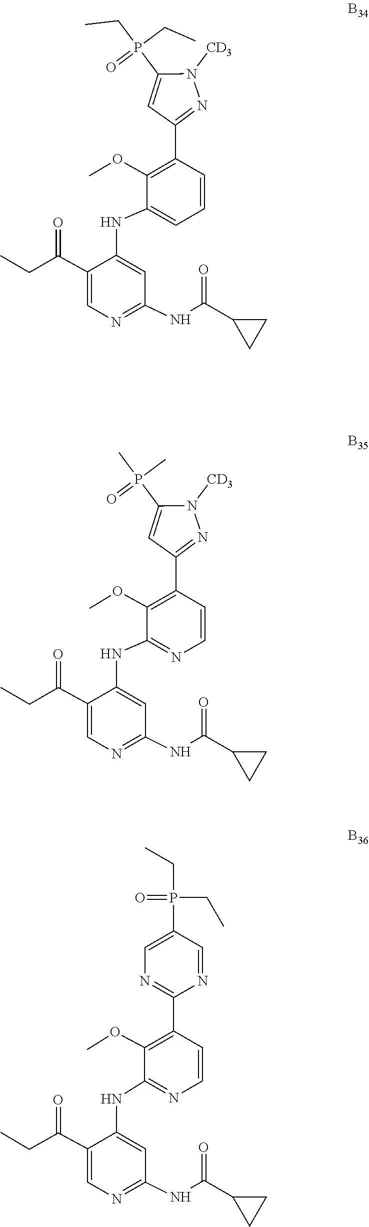 [SMILES: [2H]C([2H])([2H])n1nc(-c2cccc(Nc3cc(NC(=O)C4CC4)ncc3C(=O)CC)c2OC)cc1P(=O)(CC)CC ; [2H]C([2H])([2H])n1nc(-c2ccnc(Nc3cc(NC(=O)C4CC4)ncc3C(=O)CC)c2OC)cc1P(C)(C)=O ; CCC(=O)c1cnc(NC(=O)C2CC2)cc1Nc1nccc(-c2ncc(P(=O)(CC)CC)cn2)c1OC]

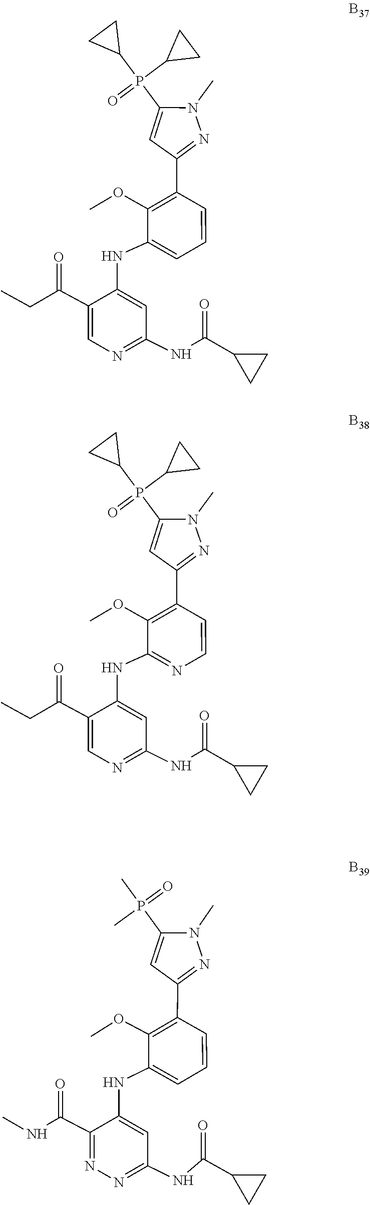 [SMILES: CCC(=O)c1cnc(NC(=O)C2CC2)cc1Nc1cccc(-c2cc(P(=O)(C3CC3)C3CC3)n(C)n2)c1OC ; CCC(=O)c1cnc(NC(=O)C2CC2)cc1Nc1nccc(-c2cc(P(=O)(C3CC3)C3CC3)n(C)n2)c1OC ; CNC(=O)c1nnc(NC(=O)C2CC2)cc1Nc1cccc(-c2cc(P(C)(C)=O)n(C)n2)c1OC]

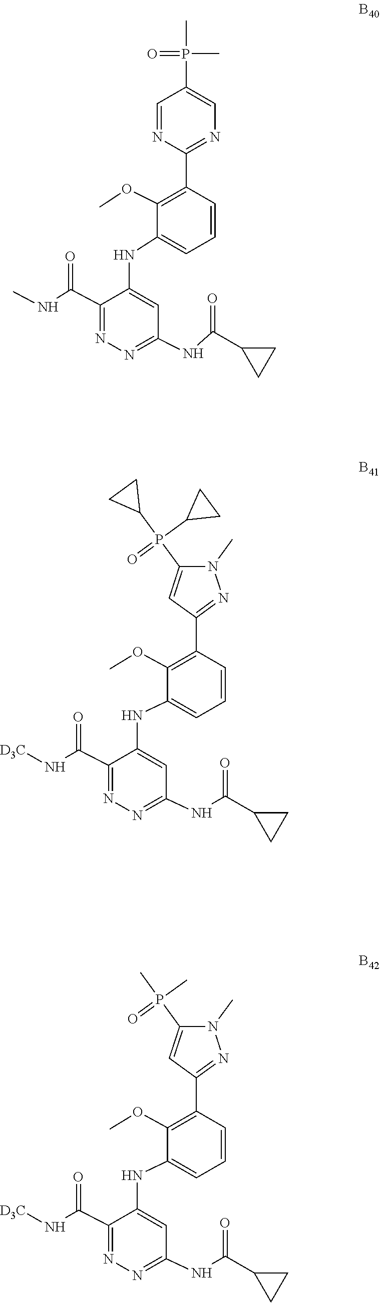 [SMILES: CNC(=O)c1nnc(NC(=O)C2CC2)cc1Nc1cccc(-c2ncc(P(C)(C)=O)cn2)c1OC ; [2H]C([2H])([2H])NC(=O)c1nnc(NC(=O)C2CC2)cc1Nc1cccc(-c2cc(P(=O)(C3CC3)C3CC3)n(C)n2)c1OC ; [2H]C([2H])([2H])NC(=O)c1nnc(NC(=O)C2CC2)cc1Nc1cccc(-c2cc(P(C)(C)=O)n(C)n2)c1OC]

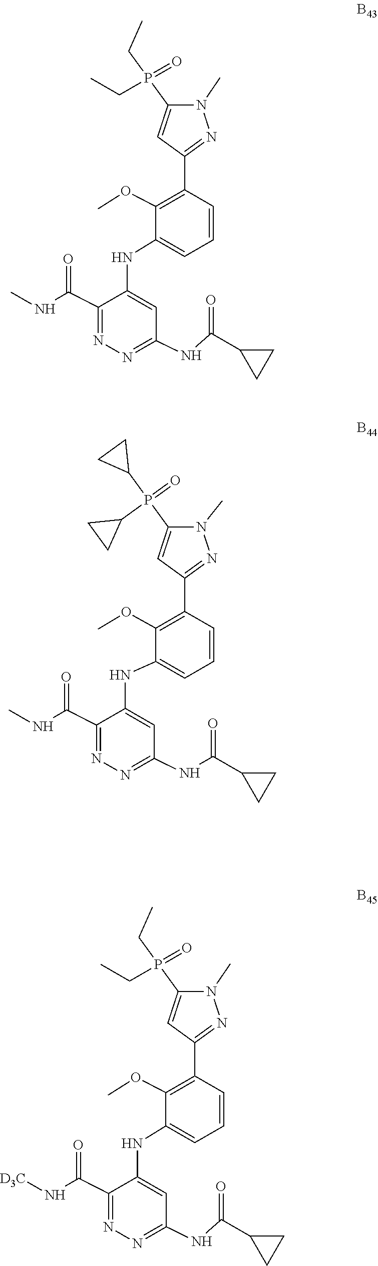 [SMILES: CCP(=O)(CC)c1cc(-c2cccc(Nc3cc(NC(=O)C4CC4)nnc3C(=O)NC)c2OC)nn1C ; CNC(=O)c1nnc(NC(=O)C2CC2)cc1Nc1cccc(-c2cc(P(=O)(C3CC3)C3CC3)n(C)n2)c1OC ; [2H]C([2H])([2H])NC(=O)c1nnc(NC(=O)C2CC2)cc1Nc1cccc(-c2cc(P(=O)(CC)CC)n(C)n2)c1OC]

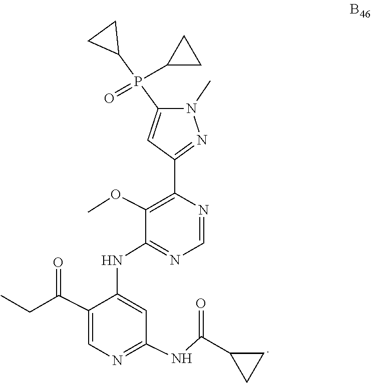 [SMILES: CCC(=O)c1cnc(NC(=O)C2CC2)cc1Nc1ncnc(-c2cc(P(=O)(C3CC3)C3CC3)n(C)n2)c1OC]

Another aspect of the present disclosure provides a pharmaceutical composition comprising a therapeutically effective amount of the compound of any one of the hitherto described embodiments, or a pharmaceutically acceptable salt, ester, solvate, prodrug, isotope-labeled derivative, or isomer thereof, and a pharmaceutically acceptable carrier.

Another aspect of the present disclosure provides a composition comprising the compound of any one of the hitherto described embodiments, or a pharmaceutically acceptable salt, ester, solvate, prodrug, isotope-labeled derivative or isomer thereof, and one or more additional therapeutic agent selected from the group consisting of anti-autoimmune/anti-inflammatory agent, anti-tumor/anti-cancer agent, anti-allergic agent, anti-transplant rejection agent, anti-neurodegenerative agent, anti-asthma agent and other anti-obstructive airway disease agent.

Another aspect of the present disclosure provides a method for treating a disease or disorder by inhibiting TYK2 mediated signal transduction in a subject suffering therefrom, comprising administering to the subject a therapeutically effective amount of the compound of any one of the hitherto described embodiments, or a pharmaceutically acceptable salt, ester, solvate, prodrug, isotope-labeled derivative or isomer thereof, or the pharmaceutical composition of any one of the hitherto described embodiments, or the composition of any one of the hitherto described embodiments, wherein the disease or disorder is autoimmune disease or inflammation disease, cancer or tumor, allergy, transplant rejection, neurodegenerative disease, asthma or other obstructive airway diseases;
wherein the autoimmune disease or inflammation disease is enteritis, skin disease, eye disease, arthritis, Hashimoto's thyroiditis, autoimmune hemolytic anemia, autoimmune atrophic gastritis, autoimmune encephalomyelitis, Goodpasture syndrome, autoimmune thrombocytopenia, sympathetic ophthalmitis, myositis, primary biliary cirrhosis, hepatitis, primary sclerosing cholangitis, chronic invasive hepatitis, nonalcoholic fatty liver disease, nonalcoholic steatohepatitis, ulcerative colitis, membranous glomerulopathy, systemic lupus erythematosus, rheumatoid arthritis, psoriatic arthritis, polyarthritis dermatomyositis, type I interferonopathies (including Aicardi-Goutières syndrome) and other systemic sclerosis caused by overexpression of type I interferon, Mendelian disease, multiple arteritis nodosa, multiple sclerosis, relapsing multiple sclerosis, primary progressive multiple sclerosis, secondary progressive multiple sclerosis and bullous pemphigus, Cogan's syndrome, ankylosing spondylitis, Wegener's granulomatosis, autoimmune alopecia, diabetes, or thyroid inflammation;
wherein the enteritis is Crohn's disease, ulcerative colitis, inflammatory bowel disease, celiac disease, proctitis, eosinophilic gastroenteritis, or mastocytosis;
wherein the skin disease is atopic dermatitis, eczema, psoriasis, scleroderma, pruritus or other symptoms of itching, vitiligo, or alopecia;
wherein the eye disease is keratoconjunctivitis, uveitis (including uveitis associated with Behçet's disease and uveitis caused by the lens), keratitis, herpetic keratitis, keratoconus, muscular dystrophic epithelial keratitis inflammation, corneal leukopenia, anterior uveitis, scleritis, Mooren's ulcer, Graves ophthalmopathy, Vogt-Koyanagi-Harada syndrome, keratoconjunctivitis sicca, vesicular, iridocyclitis iridosarcoidosis, endocrine ophthalmopathy, sympathetic ophthalmitis, allergic conjunctivitis, or ocular neovascularization;
wherein the diabetes is type 1 diabetes or diabetic complications;
wherein the cancer or tumor is digestive/gastrointestinal cancers, colon cancers, liver cancers, skin cancers (including mast cell and squamous cell carcinomas), breast cancers, ovarian cancers, prostate cancers, lymphomas, leukemia (including acute myeloid leukemia and chronic myeloid leukemia), kidney cancer, lung cancer, muscle cancer, bone cancer, bladder cancer, brain cancer, melanoma (including oral and metastatic melanoma), Kaposi's sarcoma (including multiple myeloma), myeloproliferative disorders, proliferative diabetic retinopathy, or diseases/tumors associated with vascular hyperplasia;
wherein the neurodegenerative disease is motor neuron disease, Alzheimer's disease, Parkinson's disease, amyotrophic lateral sclerosis, Huntington's disease, cerebral ischemia, neurodegenerative diseases caused by trauma, injury, glutamate neurotoxicity or hypoxia, stroke, myocardial ischemia, renal ischemia, heart disease, cardiac hypertrophy, atherosclerosis, arteriosclerosis, ischemia/reperfusion injury of organ hypoxia or platelet aggregation;
wherein the allergy is allergic dermatitis in subjects (including allergic diseases in horses, such as allergy to bites), summer eczema, itchy horseshoes, cramps, airway inflammation, recurrent airway obstruction, airway hyperresponsiveness, and chronic obstructive pulmonary disease; wherein the asthma or other obstructive airway diseases are chronic or excessive asthma, delayed asthma, bronchitis, bronchial asthma, allergic asthma, intrinsic asthma, extrinsic asthma or dusty asthma; and
wherein the transplant rejection is islet transplant rejection, bone marrow transplant rejection, graft-versus-host disease, organ and cell transplant rejection (the organ and cell are bone marrow, cartilage, cornea, heart, intervertebral disc, islet, kidney, extremity, liver, lung, muscle, myoblasts, nerves, pancreas, skin, small intestine or trachea), or xenograft rejection.
Another aspect of the present disclosure provides the compound of any one of the hitherto described embodiments, or a pharmaceutically acceptable salt, ester, solvate, prodrug, isotope-labeled derivative or isomer thereof, or the pharmaceutical composition of any one of the hitherto described embodiments, or the composition of any one of the hitherto described embodiments for use in the treatment of a disease or disorder by inhibiting TYK2 mediated signal transduction in a subject suffering therefrom,
wherein the disease or disorder is autoimmune disease or inflammation disease, cancer or tumor, allergy, transplant rejection, neurodegenerative disease, asthma or other obstructive airway diseases;
wherein the autoimmune disease or inflammation disease is enteritis, skin disease, eye disease, arthritis, Hashimoto's thyroiditis, autoimmune hemolytic anemia, autoimmune atrophic gastritis, autoimmune encephalomyelitis, Goodpasture syndrome, autoimmune thrombocytopenia, sympathetic ophthalmitis, myositis, primary biliary cirrhosis, hepatitis, primary sclerosing cholangitis, chronic invasive hepatitis, nonalcoholic fatty liver disease, nonalcoholic steatohepatitis, ulcerative colitis, membranous glomerulopathy, systemic lupus erythematosus, rheumatoid arthritis, psoriatic arthritis, polyarthritis dermatomyositis, type I Interferonopathies (including Aicardi-Goutières syndrome) and other systemic sclerosis caused by overexpression of type I interferon, Mendelian disease, multiple arteritis nodosa, multiple sclerosis, relapsing multiple sclerosis, primary progressive multiple sclerosis, secondary progressive multiple sclerosis and bullous pemphigus, Cogan's syndrome, ankylosing spondylitis, Wegener's granulomatosis, autoimmune alopecia, diabetes, or thyroid inflammation;
wherein the enteritis is Crohn's disease, ulcerative colitis, inflammatory bowel disease, celiac disease, proctitis, eosinophilic gastroenteritis, or mastocytosis;
wherein the skin disease is atopic dermatitis, eczema, psoriasis, scleroderma, pruritus or other symptoms of itching, vitiligo, or alopecia;
wherein the eye disease is keratoconjunctivitis, uveitis (including uveitis associated with Behçet's disease and uveitis caused by the lens), keratitis, herpetic keratitis, keratoconus, muscular dystrophic epithelial keratitis inflammation, corneal leukopenia, anterior uveitis, scleritis, Mooren's ulcer, Graves ophthalmopathy, Vogt-Koyanagi-Harada syndrome, keratoconjunctivitis sicca, vesicular, iridocyclitis iridosarcoidosis, endocrine ophthalmopathy, sympathetic ophthalmitis, allergic conjunctivitis, or ocular neovascularization;
wherein the diabetes is type 1 diabetes or diabetic complications;
wherein the cancer or tumor is digestive/gastrointestinal cancers, colon cancers, liver cancers, skin cancers (including mast cell and squamous cell carcinomas), breast cancers, ovarian cancers, prostate cancers, lymphomas, leukemia (including acute myeloid leukemia and chronic myeloid leukemia), kidney cancer, lung cancer, muscle cancer, bone cancer, bladder cancer, brain cancer, melanoma (including oral and metastatic melanoma), Kaposi's sarcoma (including multiple myeloma), myeloproliferative disorders, proliferative diabetic retinopathy, or diseases/tumors associated with vascular hyperplasia;
wherein the neurodegenerative disease is motor neuron disease, Alzheimer's disease, Parkinson's disease, amyotrophic lateral sclerosis, Huntington's disease, cerebral ischemia, neurodegenerative diseases caused by trauma, injury, glutamate neurotoxicity or hypoxia, stroke, myocardial ischemia, renal ischemia, heart disease, cardiac hypertrophy, atherosclerosis, arteriosclerosis, ischemia/reperfusion injury of organ hypoxia or platelet aggregation;
wherein the allergy is allergic dermatitis in subjects (including allergic diseases in horses, such as allergy to bites), summer eczema, itchy horseshoes, cramps, airway inflammation, recurrent airway obstruction, airway hyperresponsiveness, and chronic obstructive pulmonary disease; wherein the asthma or other obstructive airway diseases are chronic or excessive asthma, delayed asthma, bronchitis, bronchial asthma, allergic asthma, intrinsic asthma, extrinsic asthma or dusty asthma; and
wherein the transplant rejection is islet transplant rejection, bone marrow transplant rejection, graft-versus-host disease, organ and cell transplant rejection (the organ and cell are bone marrow, cartilage, cornea, heart, intervertebral disc, islet, kidney, extremity, liver, lung, muscle, myoblasts, nerves, pancreas, skin, small intestine or trachea), or xenograft rejection.

The present disclosure also provides a formulation of the compound disclosed herein including all embodiments, the pharmaceutical composition disclosed herein including all embodiments, or the composition disclosed herein including all embodiments, wherein the formulation is tablet, capsule, injection agent, granule, powder, suppository, pill, gel, powder, oral solution, inhalation agent, suspension, or dry suspension.

In some embodiments, including any one of the hitherto described embodiments, the disclosed compound inhibits TYK2. In some embodiments, including any one of the hitherto described embodiments, the disclosed compound inhibits both TYK2 and JAK1. In some embodiments, including any one of the hitherto described embodiments, the disclosed compound inhibits TYK2, but does not significantly inhibit JAK2 or JAK3. In some embodiments, including any one of the hitherto described embodiments, the disclosed compound inhibits TYK2 and JAK1, but does not significantly inhibit JAK2 or JAK3. In some embodiments, including any one of the hitherto described embodiments, the disclosed compound selectively inhibits TYK2, but does not significantly inhibit JAK2 or JAK3. In some embodiments, including any one of the hitherto described embodiments, the disclosed compound selectively inhibits TYK2 and JAK1, but does not significantly inhibit JAK2 or JAK3.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIG. 1 depicts the experimental results of the inhibition of TYK2 by Compound B2 according to the luciferase assay in Example 16;

FIG. 2 depicts the experimental results of the inhibition of TYK2 by Compound B2 according to ELISA in Example 15;

FIG. 3 depicts the experimental results of the selectivity assays of Compound B37 in Example 17;

FIG. 4 depicts the experimental results of the inhibition of JAK1/JAK2 pathway by compounds in Example 18;

FIG. 5 depicts the experimental results of the inhibition of TYK2/JAK2 pathway by compounds in Example 18;

FIG. 6 depicts the experimental results of the inhibition of TYK2/JAK1 pathway by compounds in Example 18;

FIG. 7 depicts the experimental results of the inhibition of JAK1/JAK2 pathway by compounds in Example 18;

FIG. 8 depicts the experimental results of the inhibition of JAK1/JAK3 pathway by compounds in Example 18;

FIG. 9 depicts the experimental results of efficacy of the compound in IL-23 induced psoriasis by Compound 41 in Example 24.

Before proceeding with the detailed description, it is to be appreciated that the following detailed description is merely exemplary in nature and is not intended to limit the invention or the application and uses thereof. Hence, although the present disclosure is, for convenience of explanation, depicted and described as shown in certain illustrative embodiments, it will be appreciated that it can be implemented in various other types of embodiments and equivalents, and in various other systems and environments. Furthermore, there is no intention to be bound by any theory presented in the preceding background or the following detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The disclosed compounds having TYK2 inhibitory activity of the present disclosure can be used as potent inhibitors of TYK2, and can be used to prevent and/or treat diseases and/or disorders related to or responsive to TYK2. More unexpectedly, some of the compounds of the present disclosure also have JAK1 inhibitory activity, can be used as effective JAK1 inhibitors, and can be used to prevent and/or treat diseases and/or conditions responsive to JAK1. In some embodiments, including any one of the hitherto described embodiments, the disclosed compound inhibits TYK2, but does not significantly inhibit JAK2 or JAK3. In some embodiments, including any one of the hitherto described embodiments, the disclosed compound inhibits TYK2 and JAK1, but does not significantly inhibit JAK2 or JAK3.

### Definitions

Compounds are generally described herein using standard nomenclature. For compounds having asymmetric centers, it should be understood that (unless otherwise specified) all of the optical isomers and mixtures thereof are encompassed. In addition, compounds with carbon-carbon double bonds may occur in Z- and E-forms, with all isomeric forms of the compounds being included in the present invention unless otherwise specified. Where a compound exists in various tautomeric forms, a recited compound is not limited to any one specific tautomer, but rather is intended to encompass all tautomeric forms.

As used herein, the singular forms “a,” “an” and “the” include plural referents unless the context clearly dictates otherwise. Thus, for example, reference to “a molecule” includes a plurality of such molecules, and the like.

The term “about” or “nearly” as used herein generally refers to within +/−15%, 10%, 9%, 8%, 7%, 6%, 5%, 4%, 3%, 2%, or 1% of the designated amount.

The term “halogen” or “halide” as used herein generally refers to fluorine, chlorine, bromine, and iodine. The term “haloalkyl” or “halo-alkyl” as used herein generally refers to an alkyl group that is substituted with one or more independently chosen halogens (e.g., “C₁-C₆ haloalkyl” groups have from 1 to 6 carbon atoms and at least one halogen). Examples of haloalkyl groups include, but are not limited to, mono-, di- or tri-fluoromethyl; mono-, di- or tri-chloromethyl; mono-, di-, tri-, tetra- or penta-fluoroethyl; mono-, di-, tri-, tetra- or penta-chloroethyl; and 1,2,2,2-tetrafluoro-1-trifluoromethyl-ethyl. The term “haloalkoxy” or “halo-alkoxy” as used herein generally refers to an alkoxy group that is substituted with one or more independently chosen halogens (e.g., “C₁-C₆ haloalkoxy” or “C₁-C₆ halo-alkoxy” groups have from 1 to 6 carbon atoms and at least one halogen attached to one of the carbon atoms). Examples of haloalkoxy groups include, but are not limited to, mono- or di-fluoromethoxy; mono- or di-chloromethoxy; mono-, di-, tri-, or tetra-fluoroethoxy; and mono-, di-, tri-, or tetra-chloroethoxy.

The term “alkyl” as used herein generally refers to a straight or branched chain saturated aliphatic hydrocarbon. Alkyl groups include groups having from 1 to 8 carbon atoms (C₁₋₈ alkyl), from 1 to 6 carbon atoms (C₁₋₆ alkyl) and from 1 to 4 carbon atoms (C₁-C₄ alkyl), including, for example, methyl, ethyl, propyl, isopropyl, n-butyl, sec-butyl, isobutyl, tert-butyl, pentyl, 2-pentyl, 3-pentyl, 2-methyl-2-butyl, 3-methyl-2-butyl, 3-methyl-1-butyl, 2-methyl-1-butyl, n-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. Similarly, C₁₋₃ alkyl refers to an alkyl group having from 1 to 3 carbon atoms in a straight or branched chain, including, for example, methyl, ethyl, propyl, and isopropyl. In some instances, a substituent of an alkyl group is specifically indicated. For example, “cyanoalkyl” refers to an alkyl group substituted with at least one cyano substituent. In some embodiments, C₁₋₆ alkyl is, preferably, methyl, ethyl, n-propyl, isopropyl or tert-butyl.

The term “alkenyl” as used herein generally refers to straight or branched chain alkene groups, which comprise at least one unsaturated carbon-carbon double bond. Alkenyl groups include C₂₋₈ alkenyl, C₂₋₆ alkenyl and C₂₋₄ alkenyl groups, which have from 2 to 8, 2 to 6 or 2 to 4 carbon atoms, respectively, including, for example, ethenyl, allyl or isopropenyl. The term “alkynyl” as used herein generally refers to straight or branched chain alkyne groups, which have one or more unsaturated carbon-carbon bonds, at least one of which is a triple bond. Alkynyl groups include C₂₋₈ alkynyl, C₂₋₆ alkynyl and C₂₋₄ alkynyl groups, which have from 2 to 8, 2 to 6 or 2 to 4 carbon atoms, respectively.

The term “alkoxy” as used herein generally refers to an alkyl group as described above attached via an oxygen bridge to another chemical moiety. Alkoxy groups include different length of the alkyl groups, such as, for example, C₁₋₆ alkoxy and C₁₄ alkoxy groups, which have from 1 to 6 or from 1 to 4 carbon atoms, respectively. The term “OC₁₋₆ alkyl” as used herein generally refers to alkoxy groups include an alkyl group (with 1 to 6 carbon atoms) attached to an oxygen atom. Methoxy, ethoxy, n-propoxy, isopropoxy, n-butoxy, sec-butoxy, isobutoxy, tert-butoxy, n-pentoxy, 2-pentoxy, 3-pentoxy, isopentoxy, neopentoxy, hexoxy, 2-hexoxy, 3-hexoxy, and 3-methylpentoxy are representative alkoxy groups.

The term “cycloalkyl” as used herein generally refers to a group that comprises one or more saturated rings in which all ring members are carbon. For example, certain cycloalkyl groups are C₃₋₈ cycloalkyl, in which the cycloalkyl group contains one or more rings having from 3 to 8 ring members, all of which are carbon, including, for example, cyclopropyl, cyclobutyl, cyclopentyl, cyclohexyl, cycloheptyl, and cyclooctyl. Other example of cycloalkyl group includes adamantyl. Cycloalkyl groups do not comprise an aromatic ring or a heterocyclic ring. The term “cycloalkenyl” as used herein generally refers to a group that comprises one or more unsaturated rings in which all ring members are carbon.

The terms “heterocyclic” or “heterocycle” or “heterocyclyl” or “cycloheteroalkyl” as used herein generally refer to a ring structure (monocycle or polycycle) containing 3-12 ring atoms (3-12 membered heterocycle), 3-8 ring atoms (3-8 membered heterocycle or 3-8 membered cycloheteroalkyl), 3-6 ring atoms (3-6 membered heterocycle or 3-6 membered cycloheteroalkyl), or 5-6 ring atoms (5-6 membered heterocycle or 5-6 membered cycloheteroalkyl), in which at least one ring atom is carbon, and at least one ring atom is heteroatom selected from N, O, and S or a heteroatom group is selected from C(═O), S(═O), and S(═O)₂. A heterocyclic group may be aromatic or non-aromatic. Piperidine and oxetane are non-limiting examples of non-aromatic heterocycles. Thiazole and pyridine are non-limiting examples of aromatic heterocycles. Other examples of heterocycle include: aziridinyl, azetidinyl, oxetanyl, pyrrolidinyl, tetrahydrofuranyl, tetrahydrothienyl, piperidinyl, morpholinyl, piperazinyl, thiomorpholinyl, tetrahydropyranyl, 1,1-dioxothiomorpholinyl, butyrolactam, valerolactam, caprolactam, butyrolactone, valerolactone and caprolactone. Similarly, the term “cycloheteroalkenyl” refers to a monocycle or polycycle ring structure comprising carbon atom(s) and heteroatom(s)/heteroatom group(s), wherein the cycloheteroalkenyl comprises at least one C═C double bond, at least one ring atom that is carbon and at least one ring atom that is heteroatom selected from N, O, and S or a heteroatom group selected from C(═O), S(═O), and S(═O)₂.

“Aryl” refers to an all-carbon monocyclic or fused-ring polycyclic groups of 6 to 12 (C₆₋₁₂ aryl) or 6 to 10 carbon atoms (C₆₋₁₀ aryl) having a completely conjugated pi-electron system. Examples, without limitation, of aryl groups are phenyl, naphthalenyl, tetrahydronaphthyl, indanyl, biphenyl, and anthracenyl. The aryl group may be substituted or unsubstituted. Typical substituents include halo, trihalomethyl, alkyl, hydroxy, alkoxy, aryloxy, mercapto, alkylthio, arylthio, cyano, nitro, carbonyl, thiocarbonyl, C-carboxy, O-carboxy, O-carbamyl, N-carbamyl, O-thiocarbamyl, N-thiocarbamyl, C-amido, N-amido, sulfinyl, sulfonyl, amino and —NR^{X}R^{Y}, wherein R^{X} and R^{Y} are independently selected from the group consisting of hydrogen, alkyl, cycloalkyl, aryl, carbonyl, acetyl, sulfonyl, trifluoromethanesulfonyl and, combined, a five- or six-membered heteroalicyclic ring. Illustrative substituted alkyl group include, but are not limited to, fluoromethyl, difluoromethyl, trifluoromethyl, aminomethyl, aminoethyl, hydoxymethyl, methoxymethyl, 2-fluoroethyl, and 2-methoxyethyl, etc.

The term “heteroaryl” as used herein generally refers to an aromatic group in which at least one aromatic ring comprises at least one heteroatom selected from N, O and S. Heteroaryls include, for example, 5-12 membered heteroaryls, 5-10 membered heteroaryls, 5-7 membered monocyclic structures or 7-12 membered bicyclic structures. The number of heteroatoms in a heteroaryl can be 1, 2, 3, 4, or more. Examples included but are not limited to thienyl, pyridyl, pyrimidinyl, pyrazinyl, pyrazolyl, pyridazinyl, pyridine-2(1H)-keto, pyridine-4(1H)-keto, pyrrolyl, pyrazolyl, thiazolyl, 1,2,3-triazolyl, 1,2,4-triazolyl, 1,2,5-oxadiazolyl, imidazolyl, furanyl, tetrazolyl, isothiazolyl, oxazolyl, isoxazolyl, thiadiazolyl, oxadiazolyl, naphthyl, benzothienyl, indolyl, benzimidazolyl, benzothiazolyl, benzofuranyl, quinolinyl, isoquinolinyl, and quinazolinyl. The heteroaryl group may be substituted or unsubstituted. Typical substituents include halo, trihalomethyl, alkyl, hydroxy, alkoxy, aryloxy, mercapto, alkylthio, arylthio, cyano, nitro, carbonyl, thiocarbonyl, C-carboxy, O-carboxy, O-carbamyl, N-carbamyl, O-thiocarbamyl, N-thiocarbamyl, C-amido, N-amido, sulfinyl, sulfonyl, amino and —NR^{X}R^{Y}, with R^{X} and R^{Y} as defined above.

The term “amino” as used herein generally refers to primary amino group (—NH₂), secondary amino group (—NH—), and tertiary amino group

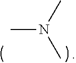

The term “alkylamino” as used herein generally refers to a secondary or tertiary amine that has the general structure —NH—R¹ or —N(R¹)(R²), respectively, wherein R¹ and R² are selected independently from alkyl, cycloalkyl and (cycloalkyl)alkyl groups. Such groups include, but are not limited to, for example, mono- and di-(C₁₋₆alkyl)amino groups, in which each C₁₋₆ alkyl may be the same or different. It will be apparent that the definition of “alkyl” as used in the term “alkylamino” differs from the definition of “alkyl” used for all other alkyl-containing groups, in the inclusion of cycloalkyl and (cycloalkyl)alkyl groups.

The term “alkylthio” as used herein generally refers to an alkyl-substituted thio group, wherein the term alkyl is as defined above.

The terms “substituent” and “substituted,” as used herein, generally denote that a molecular moiety is covalently bonded to an atom within a molecule of interest. For example, a ring substituent may be a moiety such as a halogen, alkyl group, haloalkyl group or other group that is covalently bonded to an atom (preferably a carbon or nitrogen atom) that is a ring member. Substituents of aromatic groups are generally covalently bonded to a ring carbon atom. A straight chain substituent may be a moiety such as a halogen, alkyl group, haloalkyl group or other group that is covalently bonded to an atom (preferably a carbon or nitrogen atom) that is a member of a straight chain.

The term “pharmaceutically acceptable” as used herein generally refers to a form of the compound that is safe for administration to a subject. For example, a free base, a salt form, a solvate, a hydrate, a prodrug or derivative form of a compound described herein, which has been approved for mammalian use, via oral ingestion or any other route of administration, by a governing authority or regulatory agency, such as the Food and Drug Administration (FDA) of the United States, is pharmaceutically acceptable.

Included in the compounds of Formulas (I), (II), (III), (IV), (V), (VI) and (VII) are the pharmaceutically acceptable salt forms of the free-base compounds. The term “pharmaceutically-acceptable salts” as used herein generally refers to salts, commonly used to form alkali metal salts and to form addition salts of free acids or free bases, which have been approved by a regulatory agency. Salts are formed from ionic associations, charge-charge interactions, covalent bonding, complexation, coordination, etc. The nature of the salt is not critical, provided that it is pharmaceutically acceptable.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. For example, Berge et al. describes pharmaceutically acceptable salts in detail in Pharmaceutical Sciences (1977) 66: 1-19. Pharmaceutically acceptable salts of the compounds provided herein include those derived from suitable inorganic and organic acids and bases. Inorganic acids from which salts can be derived include, but are not limited to, hydrochloric acid, hydrobromic acid, sulfuric acid, nitric acid, phosphoric acid, and the like. Organic acids from which salts can be derived include, but are not limited to, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium and other amine salt. Inorganic bases from which salts can be derived include, but are not limited to, sodium, potassium, lithium, ammonium, calcium, magnesium, iron, zinc, copper, manganese, aluminum, and the like. Organic bases from which salts can be derived include, but are not limited to, primary, secondary, and tertiary amines, substituted amines, including naturally occurring substituted amines, cyclic amines, basic ion exchange resins, and the like, examples include, but are not limited to, isopropylamine, trimethylamine, diethylamine, triethylamine, tripropylamine, and ethanolamine. In some embodiments, the pharmaceutically acceptable base addition salt is ammonium, potassium, sodium, calcium, or magnesium salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. Bis salts (i.e., two counterions) and higher salts (e.g., three or more counterions) are encompassed within the meaning of pharmaceutically acceptable salts.

As used herein, the term “ester” refers to organic compounds comprising an ester bond, including monoester, diester, trimester, and polyester.

As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Other solvates include, but are not limited to, methanol, ethanol, isopropanol, ethyl acetate, tetrahydrofuran, dimethyl sulfoxide, and N,N-dimethylformamide. Pharmaceutically acceptable solvates and hydrates are complexes that, for example, can include 1 to about 100, or 1 to about 10, or one to about 2, 3 or 4, solvent or water molecules.

As used herein, and unless otherwise specified, “prodrug” refers to a compound that can be converted under physiological conditions or by solvolysis to a biologically active compound described herein. Thus, the term “prodrug” refers to a precursor of a biologically active compound that is pharmaceutically acceptable. A prodrug can be inactive when administered to a subject, but is converted in vivo to an active compound, for example, by hydrolysis. A discussion of prodrugs is provided in Higuchi, T., et al, “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active Formulas (I), (II), (III), (IV), (V), (VI) or (VII) in vivo when such prodrug is administered to a mammalian subject. Prodrugs of an active compound, as described herein, can be prepared by modifying functional groups present in the active Formulas (I), (II), (III), (IV), (V), (VI) or (VII) in such a way that the modifications are cleaved, either in routine manipulation or in vivo, to the parent active compound. Prodrugs include compounds wherein a hydroxy, amino or mercapto group is bonded to any group that, when the prodrug of the active Formulas (I), (II), (III), (IV), (V), (VI) or (VII) is administered to a mammalian subject, cleaves to form a free hydroxy, free amino or free mercapto group, respectively.

The terms “isotope-labeled”, “isotope label”, “isotope-labeled derivative” and “isotopically labeled” refer to unnatural proportions of atomic isotopes at one or more of atoms that constitute such compounds. For example, the compounds can be radio labeled with radioactive isotopes, such as, for example, tritium (³H), iodine-125 (¹²⁵I), carbon-14 (¹⁴C). The compounds can also be isotope-labeled with ²H, ¹¹C, ¹³C, ¹⁵N, ¹⁷O, ¹⁸O, ¹⁸F, ³²P, ³⁵S, and ³⁶Cl. Certain isotope-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes can allow for ease of preparation and detectability. Further, substitution with heavier isotopes such as deuterium (i.e., H) can afford certain therapeutic advantages resulting from greater metabolic stability (e.g., increased in vivo half-life or reduced dosage requirements). Isotopically labeled disclosed compounds can generally be prepared by substituting an isotopically labeled reagent for a non-isotopically labeled reagent. In some embodiments, provided herein are compounds that can also contain unnatural proportions of atomic isotopes at one or more of atoms that constitute such compounds. All isotopic variations of compounds of the present disclosure, whether radioactive or not, are encompassed within the scope of the present disclosure.

The term “isomers” as used herein generally refers to different compounds that have the same molecular formula, including any and all geometric isomers, tautomers and stereoisomers. “Stereoisomers” are isomers that differ only in the way the atoms are arranged in space. For example, “isomers” include geometric double bond cis- and trans-isomers, also termed E- and Z-isomers; R- and S-enantiomers; diastereomers, (d)-isomers and (1)-isomers, racemic mixtures thereof; and other mixtures thereof, as falling within the scope of this disclosure, unless specified otherwise. As used herein, the term “tautomer” is a type of isomer that includes two or more interconvertible compounds resulting from at least one formal migration of a hydrogen atom and at least one change in valency (e.g., a single bond to a double bond, a triple bond to a single bond, or vice versa).

The term “each independently”, as used herein, means that at least two groups (or ring systems) present in a structure with the same or similar value ranges may have the same or different meanings under certain circumstances. For example, if substituent X and substituent Y are each independently hydrogen, halogen, hydroxyl, cyano, alkyl or aryl, then when substituent X is hydrogen, substituent Y can be hydrogen, halogen, hydroxyl, cyano, alkyl or aryl. Similarly, when the substituent Y is hydrogen, the substituent X can be hydrogen, halogen, hydroxyl, cyano, alkyl or aryl.

The terms “optional” or “optionally”, as used herein, mean that the subsequently described event or circumstance may or may not occur, and that the description includes both the occurrence and the non-occurrence of the subsequent event or circumstance.

In some embodiments, the compound(s) of Formulas (I), (II), (III), (IV), (V), (VI) or (VII) is used to treat a subject by administering the compound(s) as a pharmaceutical composition. To this end, the compound(s), in one embodiment, is combined with one or more pharmaceutically acceptable excipients, including carriers, diluents or adjuvants, to form a suitable composition, which is described in more detail herein.

The term “excipient” as used herein generally refers to any pharmaceutically acceptable additive, carrier, adjuvant, or other suitable ingredient, other than the active pharmaceutical ingredient (API), which is typically included for formulation and/or administration purposes.

The term “diluent” as used herein generally refers to an agent used as filler in order to achieve the desired composition volume or weight. The diluent may be present in the pharmaceutical composition within granules in the form of a single compound or in the form of a mixture of compounds. Non-limiting examples of diluent include lactose, starch, pregelatinized starch, microcrystalline cellulose, silicified microcrystalline cellulose, cellulose acetate, dextrose, mannitol, sodium phosphate, potassium phosphate, calcium phosphate, fructose, maltose, sorbitol, or sucrose.

The term “adjuvant,” as used herein generally refers to any substance or mixture of substances that increases the efficacy or potency of a compound disclosed herein on a target where the adjuvant is used together with the compound disclosed herein. However, when the adjuvant is used alone, no pharmacological effect is observed on the same target.

The terms “treat”, “treating,” “treatment,” and “therapy” as used herein generally refer to therapy, including without limitation, curative therapy, prophylactic therapy, and preventative therapy. Prophylactic treatment generally constitutes either preventing the onset of disorders altogether or delaying the onset of a pre-clinically evident stage of disorders in individuals. Treatment includes the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “effective amount” or “therapeutically effective amount”, as used herein, refers to a sufficient amount of an agent or a compound being administered which will relieve one or more of the symptoms of the disease or condition being treated to some extent; achieve the goal of improvement in disorder severity and the frequency of incidence over treatment of each agent by itself, the result thereof can be reduction and/or alleviation of the signs, symptoms, or causes of a disease, or any other desired alteration of a biological system; while avoiding adverse side effects typically associated with alternative therapies. For example, an “effective amount” for therapeutic uses is the amount of the composition as disclosed herein required to provide a clinically significant decrease in disease symptoms. An appropriate “effective” amount in any individual case may be determined using techniques, such as a dose escalation study. The effective amount, in one embodiment, is administered in a single dosage form or in multiple dosage forms.

Regardless of the route of administration selected, the compounds of the present invention, which may be used in a suitable hydrated form, and/or the pharmaceutical compositions of the present invention, are formulated into pharmaceutically acceptable dosage forms or by other conventional methods known to those of skill in the art.

Actual dosage levels of the active ingredients in the pharmaceutical compositions of the present invention may be varied so as to obtain an effective amount of the active ingredient to achieve the desired therapeutic response for a particular patient, composition, and mode of administration, without being toxic to the patient.

The selected dosage level will depend upon a variety of factors including the activity of the particular compound of the present invention employed, the route of administration, the time of administration, the rate of excretion of the particular compound being employed, the duration of the treatment, other drugs, compounds and/or materials used in combination with the particular hedgehog inhibitor employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient will range from about 0.0001 to about 100 mg per kilogram of body weight per day. The mode of administration can have a large effect on dosage. Higher doses may be used for localized routes of delivery.

If desired, the effective daily dose of the active compound may be administered as two, three, four, five, six or more sub-doses administered separately at appropriate intervals throughout the day, optionally, in unit dosage forms. Those of skill in the art will readily appreciate that dose levels can vary as a function of the specific compound, the severity of the symptoms and the susceptibility of the subject to side effects. Dosages for a given compound disclosed herein are readily determinable by those of skill in the art by a variety of means.

### Pharmaceutical Compositions/Formulations

One embodiment provides a pharmaceutical composition comprising a compound of Formulas (I), (II), (III), (IV), (V), (VI) or (VII), or a stereoisomer, tautomer, hydrate, solvate or pharmaceutically acceptable salt thereof, and at least one pharmaceutically acceptable excipient.

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed., Easton, Pa.: Mack Publishing Company (1995); Hoover, John E., Remington's Pharmaceutical Sciences, Mack Publishing Co., Easton, Pennsylvania (1975); Liberman, H. A. and Lachman, L., Eds., Pharmaceutical Dosage Forms, Marcel Decker, New York, N.Y. (1980); and Pharmaceutical Dosage Forms and Drug Delivery Systems, Seventh Ed., Lippincott Williams & Wilkins (1999), herein incorporated by reference for such disclosure.

A pharmaceutical composition, as used herein, refers to a mixture of a compound of Formulas (I), (II), (III), (IV), (V), (VI) or (VII) with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

All formulations for oral administration are in dosages suitable for such administration. Examples of such dosage units are tablets or capsules. In some embodiments, these contain an amount of active ingredient from about 1 to 2000 mg, advantageously from about 1 to 500 mg, and typically from about 5 to 150 mg. A suitable daily dose for a human or other mammal vary widely depending on the condition of the patient and other factors, but, once again, can be determined using routine methods and practices.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

### Synthetic Methods

Methods of the present invention may include the use of at least one compound of Formulas (I), (II), (III), (IV), (V), (VI) or (VII), which inhibits programmed necrosis in the regulation of repair and/or functional performance of a wide range of cells, tissues and organs, and have therapeutic and cosmetic applications ranging from regulation of neural tissues, bone and cartilage formation and repair, regulation of spermatogenesis, regulation of smooth muscle, regulation of lung, liver and other organs arising from the primitive gut, regulation of hematopoietic function, regulation of skin and hair growth, etc. Accordingly, the methods and compositions of the present invention include the use of the subject inhibitors for all such uses as inhibitors of programmed necrosis may be implicated. Moreover, the subject methods can be performed on cells which are provided in culture (in vitro), or on cells in a whole animal (in vivo).

The examples and preparations provided below illustrated and exemplify the compounds described herein and methods of preparing such compounds. In general, the compounds described herein may be prepared by processes known in the general chemical arts.

The compounds of the present invention can be prepared using various synthetic routes, including those described below, starting from commercially available materials. Starting materials of the invention, are either known, commercially available, or can be synthesized in analogy to or according to methods that are known in the art. Many starting materials may be prepared according to known processes and, in particular, can be prepared using processes described in the examples. In synthesizing starting materials, functional groups in some cases are protected with suitable protecting groups when necessary. Functional groups may be removed according to known procedures in the art.

The protection of functional groups by protecting groups, the protecting groups themselves, and their removal reactions (commonly referred to as “deprotection”) are described, for example, in standard reference works, such as J. F. W. McOmie, Protective Groups in Organic Chemistry, Plenum Press, London and New York (1973), in T. W. Greene, Protective Groups in Organic Synthesis, Wiley, New York (1981), in The Peptides, Volume 3, E. Gross and J. Meienhofer editors, Academic Press, London and New York (1981).

All synthetic procedures described herein can be carried out under known reaction conditions, advantageously under those described herein, either in the absence or in the presence (usually) of solvents or diluents.

The invention further encompasses “intermediate” compounds, including structures produced from the synthetic procedures described, whether isolated or not, prior to obtaining the finally desired compound. Structures resulting from carrying out steps from a transient starting material, structures resulting from divergence from the described method(s) at any stage, and structures forming starting materials under the reaction conditions are all “intermediates” included in the invention. Further, structures produced by using starting materials in the form of a reactive derivative or salt, or produced by a compound obtainable by means of the process according to the invention and structures resulting from processing the compounds of the invention in situ are also within the scope of the invention.

New starting materials and/or intermediates, as well as processes for the preparation thereof, are likewise the subject of this invention. In select embodiments, such starting materials are used and reaction conditions so selected as to obtain the desired compound(s).

Starting materials of the invention, are either known, commercially available, or can be synthesized in analogy to or according to methods that are known in the art. Many starting materials may be prepared according to known processes and, in particular, can be prepared using processes described in the examples. In synthesizing starting materials, functional groups in some cases are protected with suitable protecting groups when necessary. Protecting groups, their introduction and removal are described above.

All reagents and solvents were obtained commercially unless stated otherwise. All commercial reagents and solvent were used without purification unless stated otherwise. When required, some reagents and solvents were purified by standard techniques. For example, tetrahydrofuran may be purified by distillation from sodium. All thin-layer chromatography (TLC, GF254) analyses and column purification (100-200 mesh) were performed on silica gel (Qingdao Haiyang Chemical Co. Ltd. or Yantai Chemical Co. Ltd.), using petroleum ether (b.p. 60-90° C.)/ethyl acetate (v/v) as eluent; and spots revealed by UV visualization at 254 nm and I₂ vapor or phosphomolybdic acid. All organic layers after extraction were dried over anhydrous Na₂SO₄ unless stated otherwise. All nuclear magnetic resonance spectra (¹H NMR) were recorded using a Varian-400 spectrometer at 400 MHz using TMS as an internal standard. LC-MS was run using an Agilent 1100 system with LC-MSDTrap recorder, diode array detector (DAD) with detecting wavelength at 214 nm and 254 nm, and ESI source. The HPCL column is an AgelaDurashell C18 3.5 μm 4.6×50 mm column. Gradients were run using 0.1 NH₄HCO₃ aqueous solution and acetonitrile with gradient 5/95 to 95/5 in the run time indicated (for example, 5 min), flow rate at 1.8 mL/min.

The size and scale of the synthetic methods will vary depending on the desired amount of end product. It is understood that while specific reactants and amounts are provided in the Examples, one of skill in the art knows other alternative and equally feasible sets of reactants that will also yield the same compounds. Thus, where general oxidizers, reducers, solvents of various nature (aprotic, apolar, polar, etc.) are utilized, equivalents will be known in the art and are herein contemplated for use in the present methods.

Many of the steps below indicate various work-ups following termination of the reaction. A work-up involves generally quenching of a reaction to terminate any remaining catalytic activity and starting reagents. This is generally followed by addition of an organic solvent and separation of the aqueous layer from the organic layer. The product is typically obtained from the organic layer and unused reactants and other spurious side products and unwanted chemicals are generally trapped in the aqueous layer and discarded. The work-up in standard organic synthetic procedures found throughout the literature is generally followed by drying the product by exposure to a drying agent, such as anhydrous Na₂SO₄, to remove any excess water or aqueous byproducts remaining partially dissolved in the organic layer and concentration of the remaining organic layer. Concentration of product dissolved in solvent may be achieved by any known means, such as evaporation under pressure, evaporation under increased temperature and pressure, and the like. Such concentrating may be achieved by use of standard laboratory equipment such as rotary-evaporator distillation, and the like. This is optionally followed by one or more purification steps which may include, but is not limited to, flash column chromatography, filtration through various media and/or other preparative methods known in the art and/or crystallization/recrystallization. (See, for instance, Addison Ault, “Techniques and Experiments for Organic Chemistry,” 6th Ed., University Science Books, Sausalito, Calif., 1998, Ann B. McGuire, Ed., pp. 45-59).

### Abbreviations

DCM means dichloromethane.
DCE means 1,2-dichloroethane.
DMF means N,N-dimethylformamide.
EtOAc or EA means ethyl acetate.
MeOH means methyl alcohol.
EtOH means ethyl alcohol.
Ph₂O means diphenylether.
Dioxane is 1,4-dioxane.
Xantphos is (9,9-Dimethyl-9H-xanthene-4,5-diyl)bis(diphenylphosphane).
Pd₂(dba)₃ is tris(dibenzylideneacetone)dipalladium(0).
DEAD is diethyl azodicarboxylate.
NBS is N-bromosuccinimide.
CDI is 1,1′-carbonyldiimidazole.
THF is tetrahydrofuran.
PMBNH₂ is 4-methoxybenzylamine.
Et₃N is triethylamine.
Con. HCl or conc. HCl means concentrated hydrochloric acid.
Sol. HCl means diluted hydrochloric acid.
TLC means thin layer chromatography.
HATU means 1-[bis(dimethylamino)methylene]-1H-1,2,3-triazolo[4,5-b]pyridinium 3-oxid hexafluorophosphate.
DIPEA means diisopropylethylamine.
HPLC means high-performance liquid chromatography.
LC-MS means liquid chromatography-mass spectrometry.
NMR means nuclear magnetic resonance.

### General Synthetic Routes

The following Methods AA-AN are embodiments for some general synthetic routes leading to compounds of Formulas (I), (II), (III), (IV), (V), (VI) or (VII). Detailed reaction conditions for each Method can be found in the examples shown vide infra.

### Method AA:

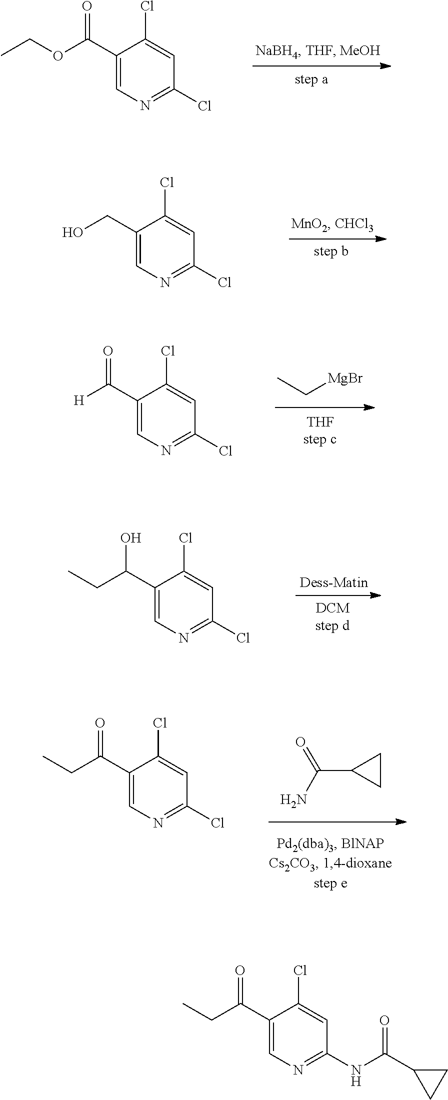

Reduction of ethyl 4,6-dichloronicotinate yielded corresponding alcohol which was oxidized by MnO₂ to give aldehyde (steps a, b). Nucleophilic substitution reaction on the aldehyde was carried out using ethylmagnesium bromide to give the alcohol, which was subsequently oxidized by Dess-Martin Reaction (steps c, d). Palladium-catalyzed Buchwald-Hartwig reaction yielded the desired compound (step e).

### Method AB

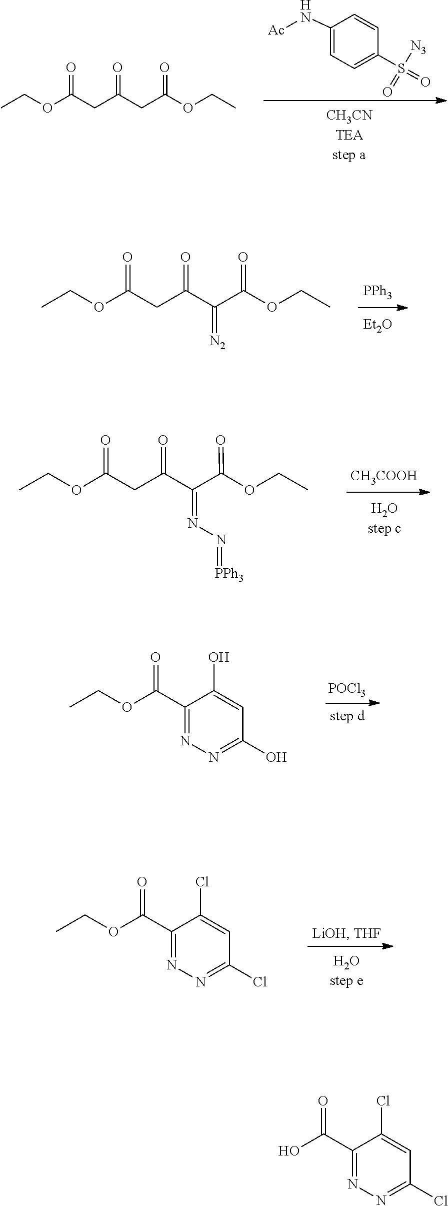

Regitz diazo transfer reaction of diethyl 3-oxopentanedioate was carried out with arylsulfonyl azides (step a). Treatment with triphenylphosphine followed by cyclization reaction yielded pyrimidine derivative (steps b, c). Chlorination of OH group with phosphorus oxychloride followed by ester hydrolysis provided the final compound (steps d, e).

### Method AC

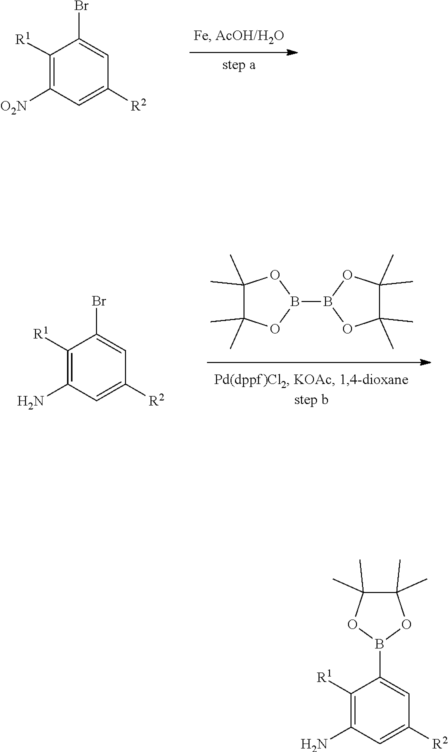

Reduction of the nitro group with iron powder followed by cross-coupling reaction gave the corresponding compound (steps a, b).

### Method AD

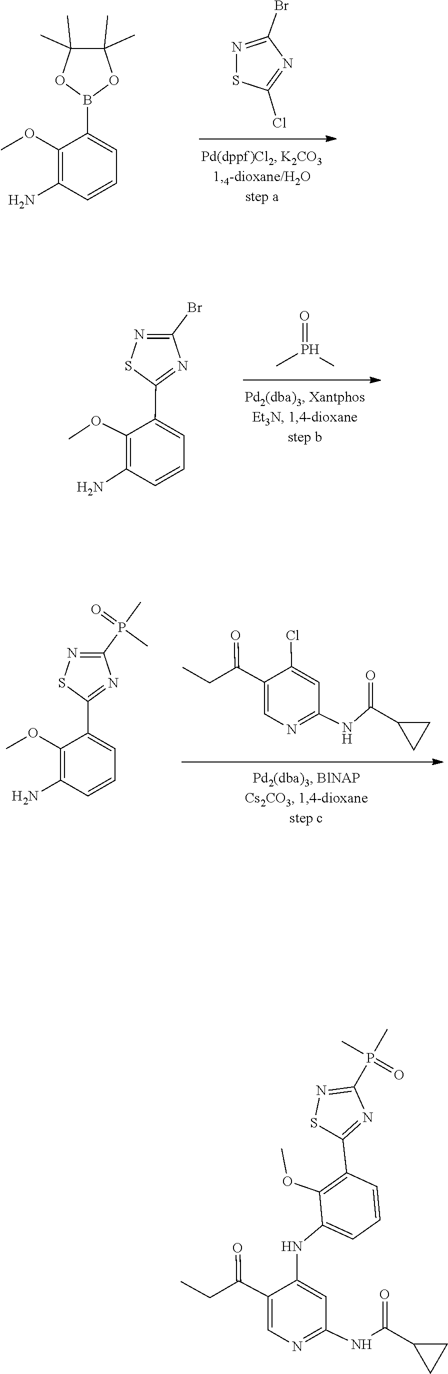

Palladium-catalyzed Suzuki reaction followed by cross-coupling reaction with dimethylphosphine oxide yielded the corresponding intermediate (steps a, b), which was coupled with N-(4-chloro-5-propionylpyridin-2-yl)cyclopropanecarboxamide to give the desired compound (step c).

### Method AE

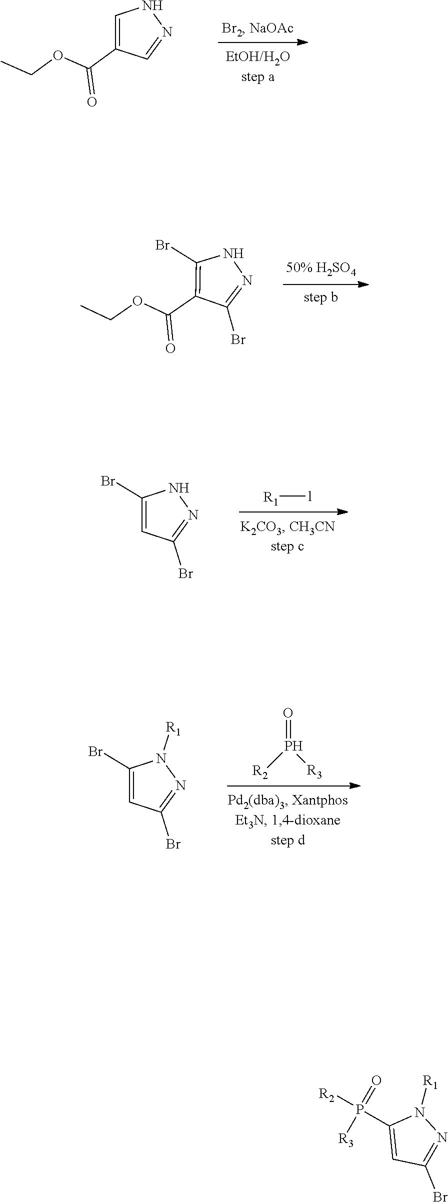

Bromination of ethyl 1H-pyrazole-4-carboxylate with bromine followed by decarboxylation with 50% H₂SO₄ yielded 3,5-dibromo-1H-pyrazole (steps a, b). After alkylation under basic conditions (step c), the intermediates were coupled with alkyl phosphorus oxide to give the desired products (step d)).

### Method AF

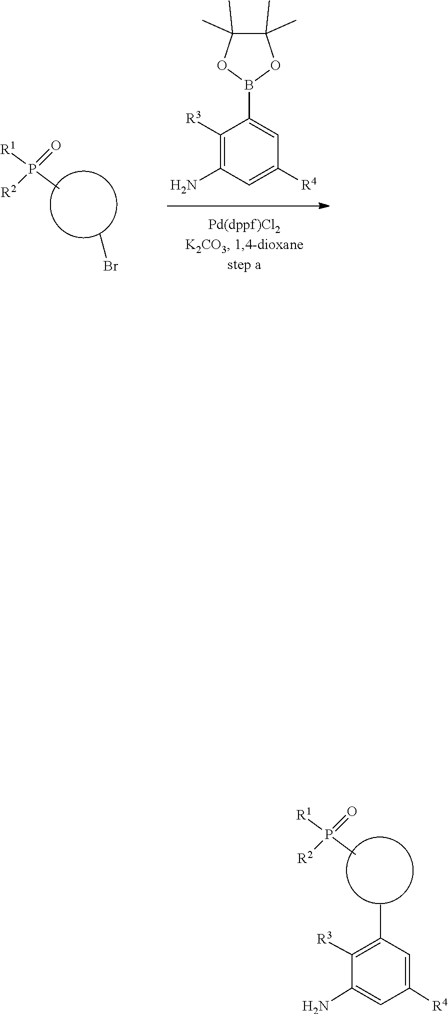

The desired products were obtained through Suzuki reaction (step a).

### Method AG

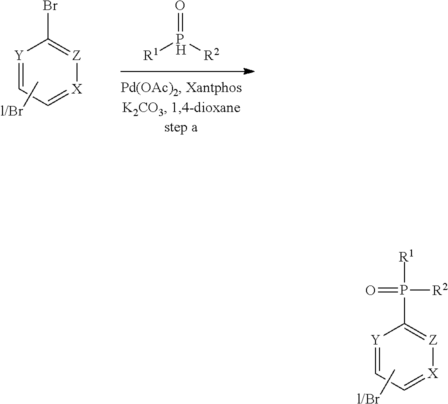

The various aryl halidesorheterocyclyl halides were coupled with alkyl phosphorus oxide to give the desired products (step a).

### Method AH

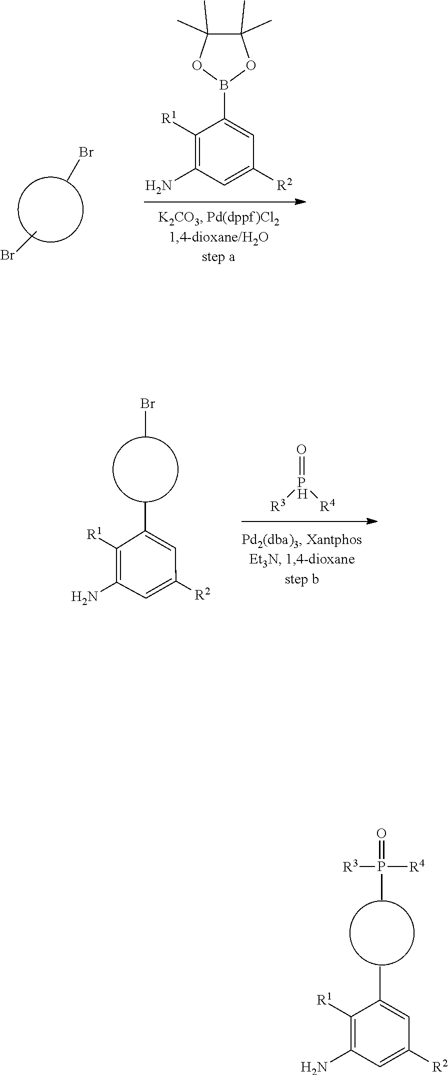

The desired compounds were obtained through Suzuki reaction and Buchwald-Hartwig reaction (steps a, b).

### Method AI

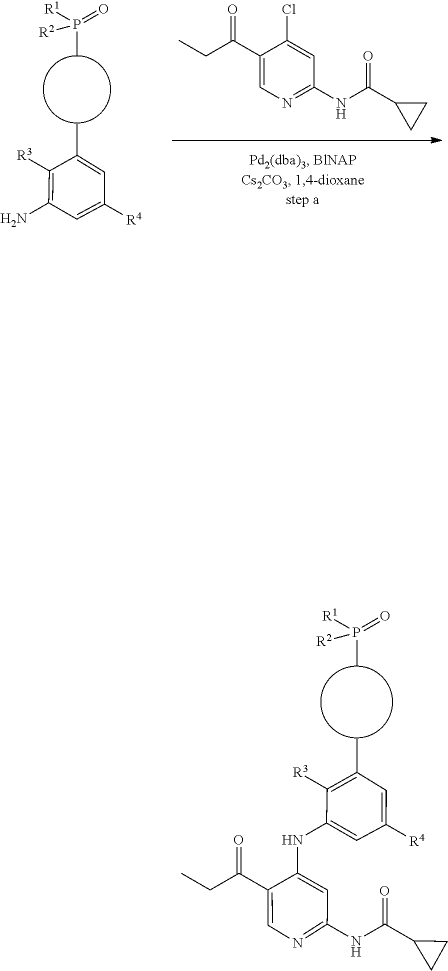

The final compounds were obtained through Buchwald-Hartwig reaction (step a).

### Method AJ

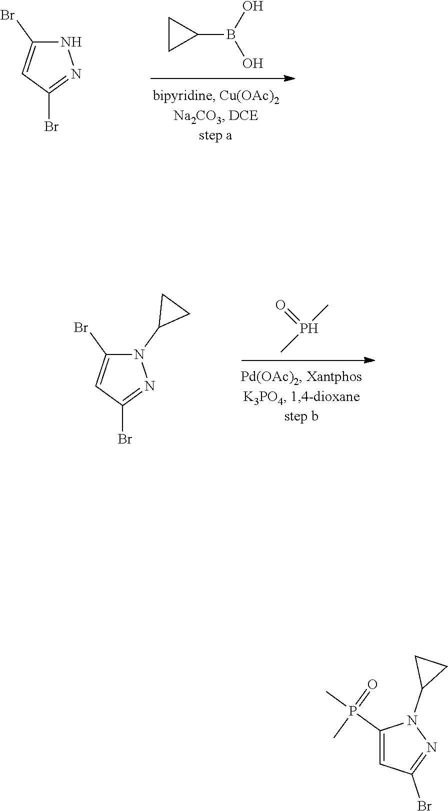

The desired compound was obtained through copper-mediated coupling followed by Buchwald-Hartwig reaction (step a, b).

### Method AK

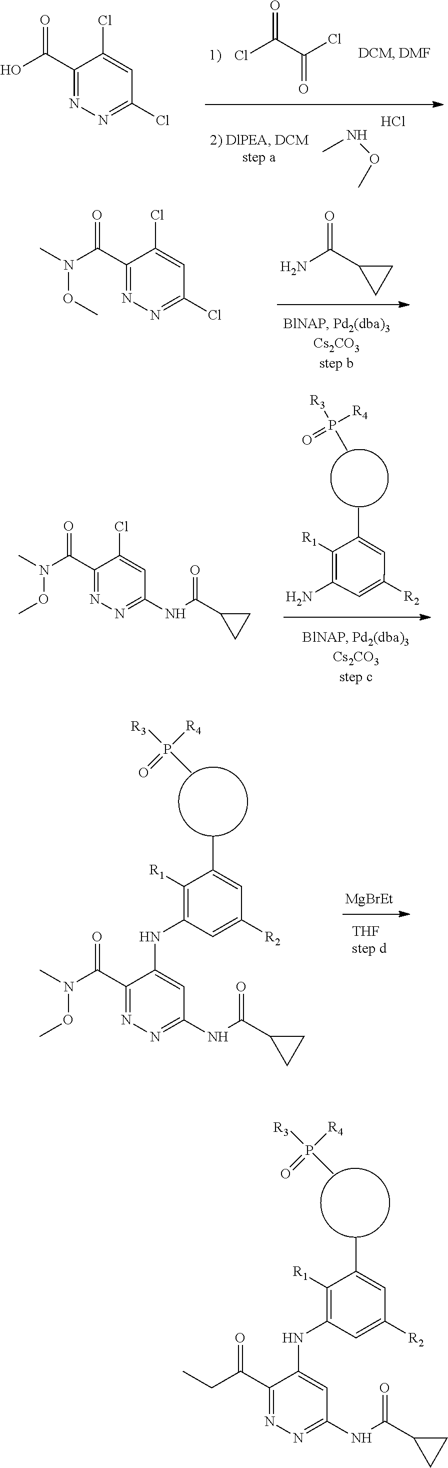

Activation of the carboxylic acid of 4,6-dichloropyridazine-3-carboxylic acid using oxalyl chloride facilitated substitution to form the corresponding Weinreb amide (step a). Palladium-catalyzed Buchwald-Hartwig reactions followed by nucleophilic reaction of amide with ethylmagnesium bromide produced the final compounds (steps b, c, d).

### Method AL

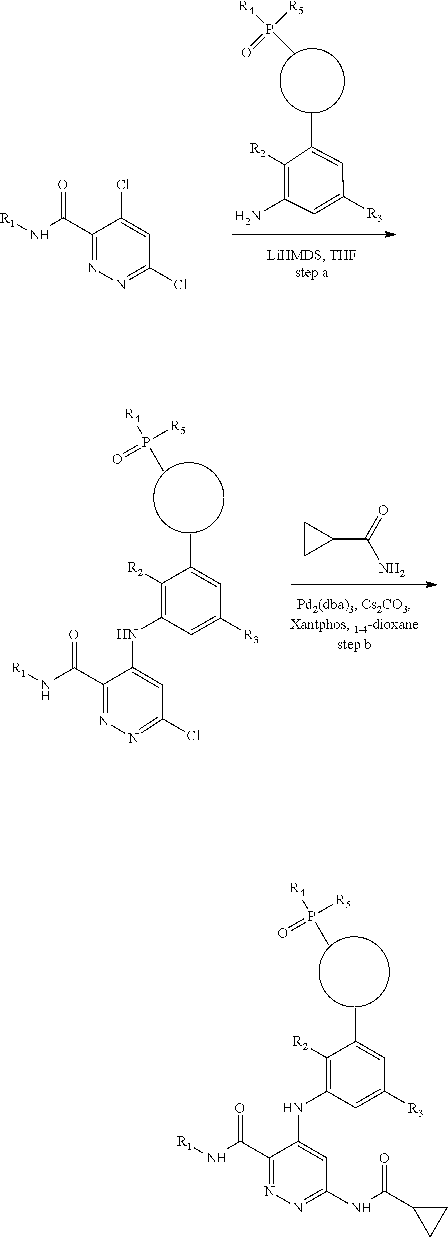

S_{N}Ar reaction was carried out under basic conditions (step a). Palladium-catalyzed Buchwald-Hartwig reaction yielded the desired compounds (step b).

### Method AM

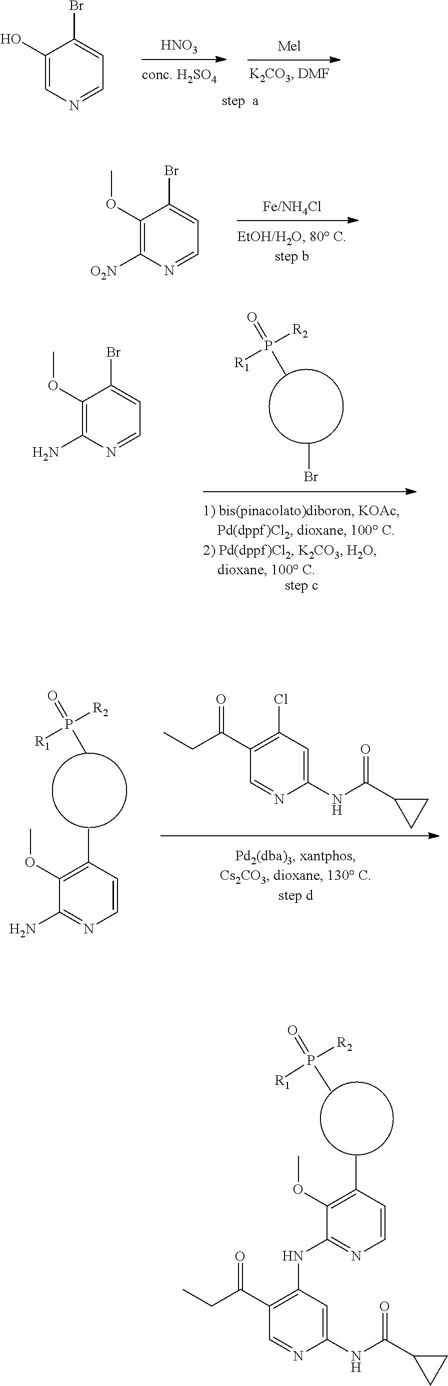

Nitration of 4-bromopyridin-3-ol with HNO₃ and methylation of OH group with iodomethane gave the corresponding intermediate (step a). Treatment with iron powder yielded pyridin-2-amine derivative (step b). Which was coupled with bis(pinacolato)diboron followed by Suzuki reaction and Buchwald-Hartwig reaction to give the desired compounds (steps c, d).

### Method AN

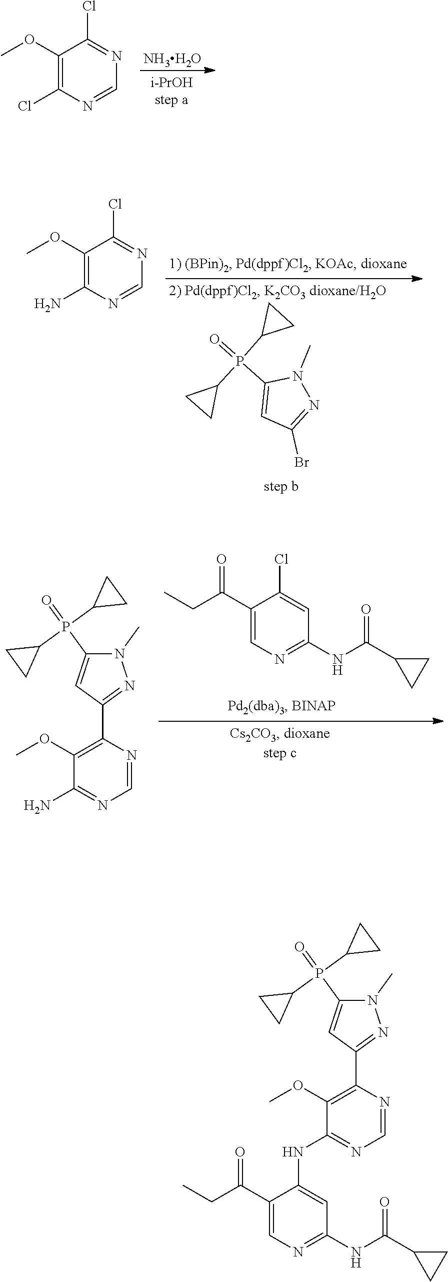

Treatment of 4,6-dichloro-5-methoxypyrimidine with ammonium hydroxide obtained the corresponding intermediate (step a). Palladium-catalyzed Suzuki reaction followed by Buchwald-Hartwig gave the final compound (step b, c).

### Examples

General reaction progress was monitored by analytical thin layer chromatography performed on silica gel HSGF254 pre-coated plates. Organic solutions were dried over anhydrous Na₂SO₄, and the solvents were removed under reduced pressure. Final compounds were purified with silica gel 100-200 mesh for column chromatography. 1H NMR were obtained on 400 MHZ (Varian) spectrometer, and 13C NMR were obtained on 151 MHz or 101 MHz (Varian) spectrometer. Chemical shifts were given in ppm using tetramethylsilane as internal standard. Mass spectra were obtained using an Agilent 1100 LC/MSD Trap SL version Mass Spectrometer. HRMS analysis was recorded on an Agilent 6540 UHD Accurate-Mass Q-TOF LC/MS.

### Example 1, Method AA

### Preparation of N-(4-chloro-5-propionylpyridin-2-yl)cyclopropanecarboxamide

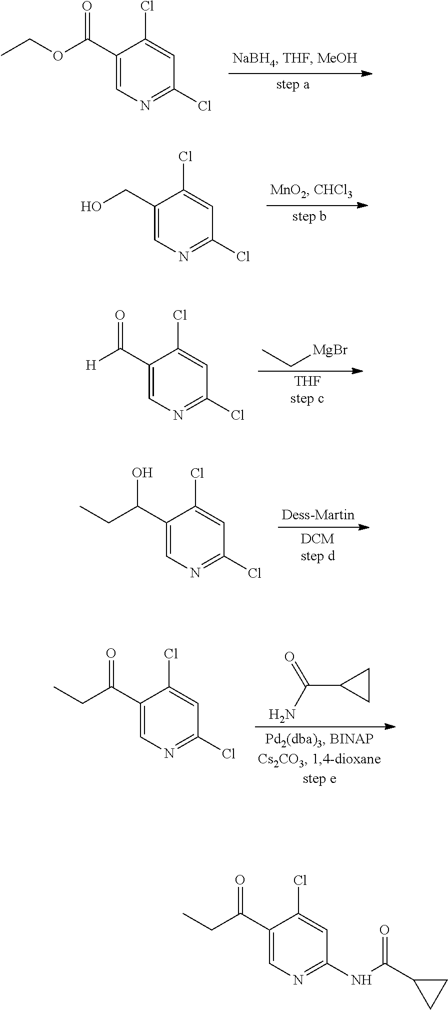

Step a. (4,6-Dichloropyridin-3-yl)methanol: To a solution of ethyl 4,6-dichloronicotinate (5.0 g, 22 mmol) in tetrahydrofuran/methanol (80 mL/20 mL) was added NaBH₄ (8.6 g, 56 mmol) slowly at 0° C. The mixture was stirred at room temperature for 3 hours. Saturated NH₄Cl aqueous solution was added to quench the reaction. The aqueous layer was extracted by ethyl acetate (100 mL×2). The organic layers were combined and concentrated to give the crude product (4.0 g, 92%) as a yellow solid. 1H NMR (400 MHZ, CDCl₃): δ 8.48 (s, 1H), 7.38 (s, 1H), 4.81 (s, 2H). LC-MS: m/z 178.0 [M+H]⁺.

Step b. 4,6-Dichloronicotinaldehyde: To a solution of (4,6-dichloropyridin-3-yl)methanol (15 g, 84 mmol) in CHCl₃ (100 mL) was added MnO₂ (78 g, 842 mmol). The mixture was stirred at 75° C. overnight. The solvent was filtered and the filtrate was concentrated. The residue was purified by silica gel chromatography column by (PE/EA=15/1) to give the desired product (7.0 g, 47%) as a white solid. 1H NMR (400 MHZ, CDCl₃): δ 10.44 (s, 1H), 8.85 (s, 1H), 7.50 (s, 1H).

Step c. 1-(4,6-Dichloropyridin-3-yl)propan-1-ol: To a solution of 4,6-dichloronicotinaldehyde (3.0 g, 17 mmol) in tetrahydrofuran (30 mL) was added ethylmagnesium bromide (5.6 mL, 22 mmol) at −20° C. under N₂ atmosphere. The mixture was stirred at −20° C. for 10 minutes and room temperature for 30 minutes. Saturated NH₄Cl aqueous solution was added to quench the solution. Saturated NaCl aqueous solution (20 mL) was added and the aqueous layer was extracted by ethyl acetate (30 ml×2). The organic layers were combined and concentrated. The residue was purified by silica gel chromatography column by (PE/EA=15/1) to give the desired product (1.5 g, 42%) as a yellow oil. ¹H NMR (400 MHZ, CDCl₃): δ 8.53 (s, 1H), 7.33 (s, 1H), 5.03 (s, 1H), 2.15 (br. s, 1H), 1.87-1.72 (m, 2H), 1.01 (t, J=7.2 Hz, 3H). LC-MS: m/z 206.0[M+H]⁺.

Step d. 1-(4,6-Dichloropyridin-3-yl)propan-1-one: To a solution of 1-(4,6-dichloropyridin-3-yl)propan-1-ol (4.2 g, 20 mmol) in dichloromethane (120 mL) was added Dess-Martin reagent (13 g, 31 mmol). The mixture was stirred at room temperature for 3 hours. Saturated NaHCO₃ aqueous solution (30 mL) was added to quench the solution. The organic layer was separated and concentrated. The residue was purified by silica gel chromatography column by (PE/EA=15/1) to give the desired product (3.3 g, 79%) as a yellow solid. ¹H NMR (400 MHZ, CDCl₃): δ 8.54 (s, 1H), 7.45 (s, 1H), 2.98 (q, J=7.2 Hz, 2H), 1.23 (t, J=7.2 Hz, 3H).LC-MS: m/z 204.0[M+H]⁺.

Step e. N-(4-Chloro-5-propionylpyridin-2-yl)cyclopropanecarboxamide: To a solution of 1-(4,6-dichloropyridin-3-yl)propan-1-one (2.2 g, 10 mmol) in 1,4-dioxane (10 mL) was added cyclopropanecarboxamide (850 mg, 10 mmol), Cs₂CO₃ (5.2 g, 15 mmol), BINAP (291 mg, 0.32 mmol), Pd₂(dba)₃ (330 mg, 0.53 mmol). The mixture was stirred at 115° C. under N₂ atmosphere in a microwave apparatus. Dichloromethane (20 mL) was added to dilute the solution and the solvent was filtered. The filtrate was concentrated and the residue was purified by silica gel chromatography column (PE/EA=10/1) to give the desired product (1.0 g, 37%) as a yellow solid. ¹H NMR (400 MHZ, CDCl₃): δ 8.50 (s, 1H), 8.35 (s, 1H), 8.22 (s, 1H), 3.00 (q, J=6.8 Hz, 2H), 1.56-1.53 (m, 1H), 1.21 (t, J=7.2 Hz, 3H), 1.14 (s, 2H), 0.96 (d, J=5.2 Hz, 2H).LC-MS: m/z 253.1 [M+H]⁺.

### Example 2, Method AB

### Preparation of 4,6-dichloropyridazine-3-carboxylic acid

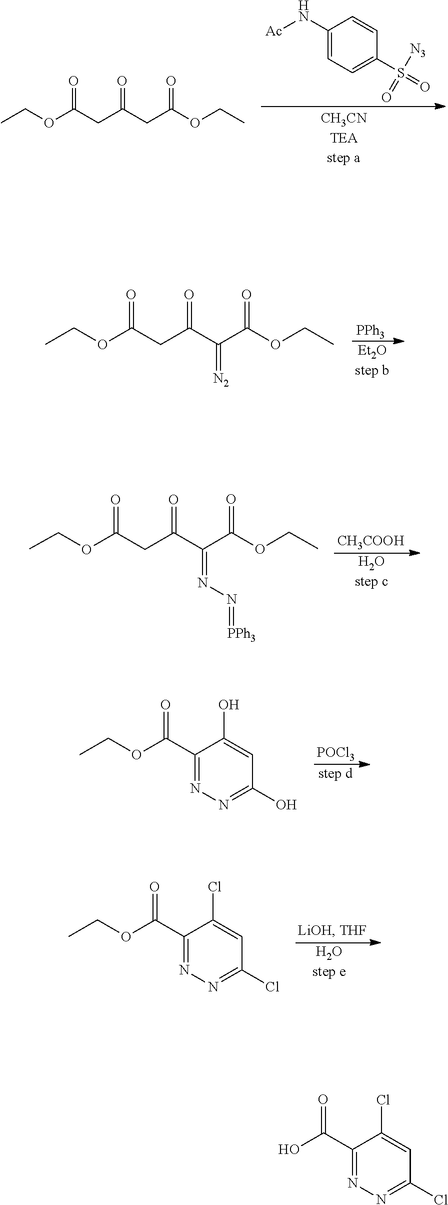

Step a. Diethyl 2-diazo-3-oxopentanedioate: To a solution of diethyl 3-oxopentanedioate (7.0 g, 34.6 mmol) in acetonitrile (100 mL) was added Et₃N (3.8 g, 38.1 mmol) and 4-acetamidobenzenesulfonyl azide (8.7 g, 36.9 mmol) at 0° C. The mixture was stirred at room temperature for 1 hour. The solvent was filtered and the filtrate was concentrated. The residue was dissolved in ethyl ether (200 mL) and the solution was filtered again. The filtrate was concentrated to give the crude product (7.4 g, 94%) as a yellow solid.

Step b. Diethyl 3-oxo-2-((triphenyl-15-phosphanylidene)hydrazono)pentanedioate: To a solution of diethyl 2-diazo-3-oxopentanedioate (7.4 g, 32.5 mmol) in ethyl ether (250 mL) was added PPh₃ (9.6 g, 36.5 mmol). The mixture was stirred at room temperature for 48 hours. The solvent was concentrated to give the crude product (17.0 g, 94%) as a yellow oil.

Step c. Ethyl 4,6-dihydroxypyridazine-3-carboxylate: The diethyl 3-oxo-2-((triphenyl-15-phosphanylidene)hydrazono)pentanedioate (17.0 g, 34.7 mmol) was dissolved in acetic acid/water (80 mL/8 mL). The mixture was stirred at reflux for 12 hours. The solvent was removed and the residue was rinsed by ethyl acetate (50 mL) to give the desired product (3.0 g, 47%) as a white solid. ¹H NMR (300 MHZ, CDCl₃): δ 12.30 (s, 1H), 10.62 (s, 1H), 6.33 (s, 1H), 4.53 (q, J=7.2 Hz, 2H), 1.49 (t, J=7.2 Hz, 3H).LC-MS: 185.1 [M+H]⁺.

Step d. ethyl 4,6-dichloropyridazine-3-carboxylate: The ethyl 4,6-dihydroxypyridazine-3-carboxylate (21 g, 114.1 mmol) was dissolved in POCl₃ (250 mL). The mixture was stirred at 115° C. overnight. The solvent was removed and saturated NaCl aqueous solution (200 mL) was added. The aqueous layer was extracted by ethyl acetate (200 mL×3). The organic layers were combined and concentrated. The residue was purified by silica gel chromatography column by (PE/EA=10/1) to give the desired product (10.6 g, 42%) as a brown oil.

Step e. 4,6-dichloropyridazine-3-carboxylic acid: To a solution of ethyl 4,6-dichloropyridazine-3-carboxylate in tetrahydrofuran (100 mL) was added IN LiOH (92 mL, 92 mmol). The mixture was stirred at room temperature for 1 hour. The solvent was removed and 1.5 N HCl was added to adjust pH to 2. The aqueous layer was extracted by ethyl acetate (200 mL). The organic layer was dried by Na₂SO₄ and concentrated to give the crude product (10.6 g, 90%) as a yellow solid.

### Example 3, Method AC

### Prepare of 2-methoxy-3-(4,4,5,5-tetramethyl-1,3,2-dioxaborolan-2-yl)aniline

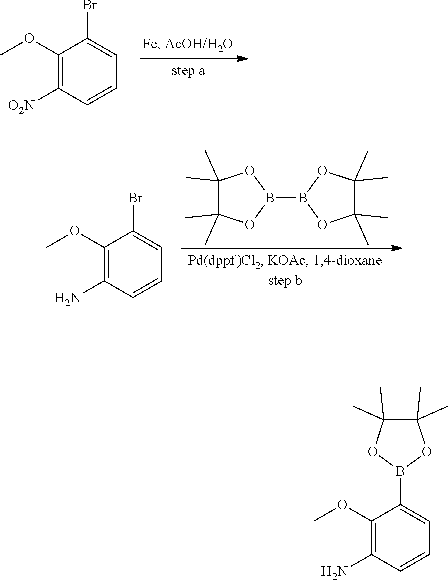

Step a. 3-bromo-2-methoxyaniline: To a solution of 1-bromo-2-methoxy-3-nitrobenzene (8.2 g, 35 mmol) in acetic acid/water (45 mL/45 mL) was added iron powder (14 g, 25 mmol). The mixture was stirred at 80° C. for 2 hours. The solution was filtered via diatomite and the cake was washed with dichloromethane (200 mL). The filtrate was concentrated. Water (100 mL) and dichloromethane (100 mL) was added. Saturated NaHCO₃ aqueous solution was used to adjust pH to 8. The aqueous layer was extracted by dichloromethane (100 mL×2). The organic layers were combined, dried by Na₂SO₄ and concentrated to give the desired product (6.9 g, 95%) as a brown oil. ¹H NMR (400 MHZ, DMSO-d₆): δ 6.72 (d, J=7.6 Hz, 1H), 6.69 (br. s, 1H), 6.67 (br. s, 1H), 5.23 (s, 2H), 3.66 (s, 3H).LC-MS: 202.0 [M+H]⁺.

Step b. 2-methoxy-3-(4,4,5,5-tetramethyl-1,3,2-dioxaborolan-2-yl)aniline: To a solution of 3-bromo-2-methoxyaniline (6.1 g, 30 mmol) in 1,4-dioxane (60 mL) was added (Bpin)₂ (11.4 g, 45 mmol), KOAc (8.8 g, 90 mmol) and Pd(dppf)Cl₂ (1.3 g, 1.8 mmol). The mixture was stirred at 100° C. under N₂ atmosphere overnight. The solvent was removed and the residue was purified by silica gel chromatography column (PE/EA=4/1) to give the final compound (7.0 g, 93%) as a yellow oil. ¹H NMR (300 MHZ, CDCl₃): δ 7.11 (d, J=6.9 Hz, 1H), 6.93 (t, J=7.5 Hz, 1H), 6.85 (d, J=7.2 Hz, 1H), 3.81 (s, 3H), 1.36 (s, 12H).LC-MS: 250.1 [M+H]⁺.

### Example 4, Method AD

### Preparation of N-(4-((3-(3-(dimethylphosphoryl)-1,2,4-thiadiazol-5-yl)-2-methoxyphenyl)amino)-5-propionylpyridin-2-yl)cyclopropanecarboxamide (B1)

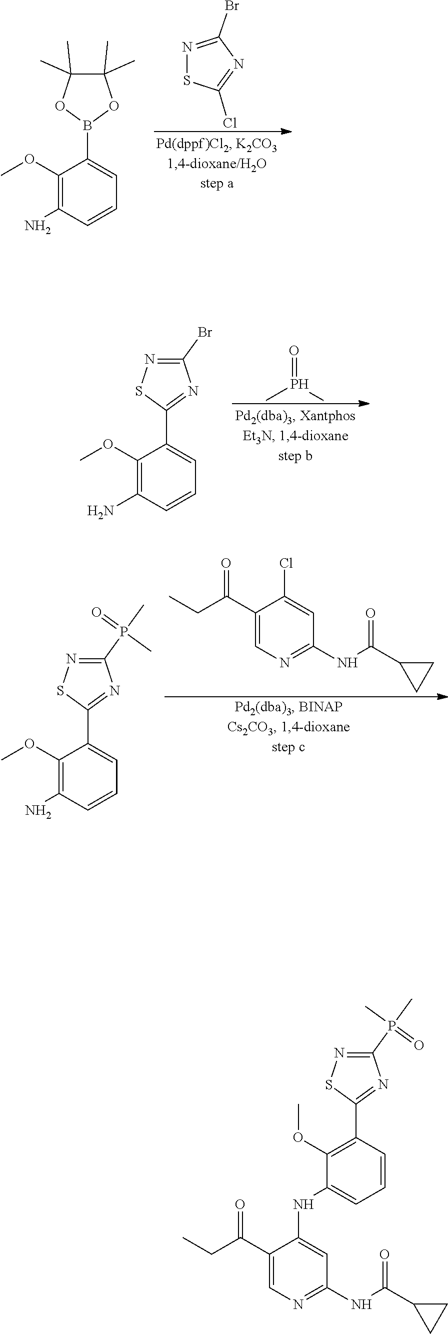

Step a. 3-(3-Bromo-1,2,4-thiadiazol-5-yl)-2-methoxyaniline: To a solution of 2-methoxy-3-(4,4,5,5-tetramethyl-1,3,2-dioxaborolan-2-yl)aniline (423 mg, 1.7 mmol) and 3-bromo-5-chloro-1,2,4-thiadiazole (389 mg, 1.7 mmol) in 1,4-dioxane/water (8 mL/1 mL) was added Pd(dppf)Cl₂ (66 mg, 0.09 mmol) and K₂CO₃ (469 mg, 3.4 mmol). The mixture was stirred at 110° C. under N₂ atmosphere for 4 hours. The solvent was removed and the residue was purified by silica gel chromatography column (PE/EA=5/1) to give the final compound (218 mg, 45%) as a white solid. ¹H NMR (300 MHz, DMSO-d₆): δ 7.36 (d, J=7.6 Hz, 1H), 7.06 (t, J=7.6 Hz, 1H), 6.96 (d, J=7.6 Hz, 1H), 5.45 (s, 2H), 3.83 (s, 3H).LC-MS: 286.0 [M+H]⁺.

Step b. (5-(3-Amino-2-methoxyphenyl)-1,2,4-thiadiazol-3-yl)dimethylphosphine oxide: To a solution of 3-(3-bromo-1,2,4-thiadiazol-5-yl)-2-methoxyaniline (218 mg, 0.76 mmol) in 1,4-dioxane (8 mL) was added dimethylphosphine oxide (179 mg, 2.29 mmol), Pd₂(dba)₃ (73 mg, 0.08 mmol), Xantphos (46 mg, 0.08 mmol) and Et₃N (154 mg, 1.52 mmol). The mixture was stirred at 130° C. under N₂ atmosphere for 2.5 hours in a microwave apparatus. The solvent was removed and the residue was purified by silica gel chromatography column (DCM/MeOH=25/1) to give the final compound as a yellow solid (90 mg, 42%). ¹H NMR (400 MHz, CDCl₃): δ 7.77 (d, J=8.0 Hz, 1H), 7.07 (t, J=8.0 Hz, 1H), 6.92 (d, J=8.0 Hz, 1H), 3.96 (s, 2H), 3.88 (s, 3H), 1.97 (s, 3H), 1.93 (s, 3H).LC-MS: 284.0 [M+H]⁺.

Step c. N-(4-((3-(3-(Dimethylphosphoryl)-1,2,4-thiadiazol-5-yl)-2-methoxyphenyl)amino)-5-propionylpyridin-2-yl)cyclopropanecarboxamide (B1): To a solution of (5-(3-amino-2-methoxyphenyl)-1,2,4-thiadiazol-3-yl)dimethylphosphine oxide (60 mg, 0.21 mmol) and N-(4-chloro-5-propionylpyridin-2-yl)cyclopropanecarboxamide (58 mg, 0.23 mmol) in 1,4-dioxane (4 mL) was added Pd₂(dba)₃ (18 mg, 0.02 mmol), BINAP (12 mg, 0.02 mmol) and Cs₂CO₃ (137 mg, 0.42 mmol). The mixture was stirred at 130° C. under N₂ atmosphere in a microwave apparatus for 2 hours. The solvent was concentrated and the residue was purified by silica gel chromatography column (DCM/MeOH=100/3) to give the final compound as a yellow solid (20 mg, 19%).

### Example 5, Method AE

### Preparation of (3-bromo-1-methyl-1H-pyrazol-5-yl)dimethylphosphine oxide

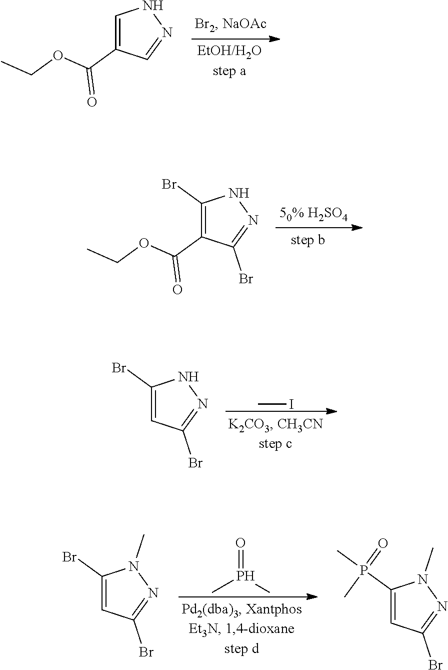

Step a. Ethyl 3,5-dibromo-1H-pyrazole-4-carboxylate: To a solution of ethyl 1H-pyrazole-4-carboxylate (3.0 g, 21.3 mmol) in ethanol/water (18 mL/27 mL) was added NaOAc (6.9 g, 95.0 mmol) and bromide (8.4 g, 53.0 mmol) dropwise. The mixture was stirred at room temperature for 4 hours. Saturated Na₂SO₃ aqueous solution (60 mL) was added to quench the reaction. The aqueous layer was extracted by dichloromethane (60 mL×3). The organic layers were combined, dried by Na₂SO₄ and concentrated to give the desired compound (6.1 g, 96%) as a white solid. ¹H NMR (400 MHz, CDCl₃): δ 4.37-4.19 (m, 2H), 1.42-1.23 (m, 3H). LC-MS: 296.9 [M+H]⁺.

Step b. 3,5-Dibromo-1H-pyrazole: Ethyl 3,5-dibromo-1H-pyrazole-4-carboxylate (3.0 g, 10.0 mmol) was dissolved in 50% H₂SO₄ (30 mL). The solution was stirred at 160° C. for 2 hours. After cooling to room temperature, Saturated NaHCO₃ aqueous solution (100 mL) was added to neutralize the acid. The aqueous layer was extracted by ethyl acetate (20 mL×4). The organic layers were combined, dried by Na₂SO₄ and concentrated. The residue was purified by silica gel chromatography column (PE/EA=10/1) to give the final compound (1.4 g, 62%) as a white solid. ¹H NMR (300 MHz, DMSO-d₆): δ 13.92 (s, 1H), 6.58 (s, 1H). LC-MS: 225.0 [M+H]⁺.

Step c. 3,5-Dibromo-1-methyl-1H-pyrazole: To a solution of 3,5-dibromo-1H-pyrazole (500 mg, 2.2 mmol) in acetonitrile (10 mL) was added K₂CO₃ and iodomethane (369 mg, 2.6 mmol). The mixture was stirred at 80° C. overnight. The solvent was removed and the residue was purified by silica gel chromatography column (PE/EA=10/1) to give the final compound (350 mg, 66%). ¹H NMR (400 MHZ, CDCl₃): δ 6.29 (s, 1H), 3.84 (s, 3H). LC-MS: 239.0 [M+H]⁺.

Step d. (3-bromo-1-methyl-1H-pyrazol-5-yl)dimethylphosphine oxide: To a solution of 3,5-dibromo-1-methyl-1H-pyrazole (250 mg, 1.04 mmol) in 1,4-dioxane (5 mL) was added Et₃N (210 mg, 2.08 mmol), dimethylphosphine oxide (122 mg, 1.56 mmol), Pd₂(dba)₃ (92 mg, 0.10 mmol) and Xantphos (58 mg, 0.10 mmol). The mixture was stirred at reflux under N₂ atmosphere for 24 hours. The solvent was concentrated and the residue was purified by silica gel chromatography column (PE/EA=10/1) to give the final compound (90 mg, 37%) as a light yellow oil. ¹H NMR (400 MHZ, CDCl₃): δ 6.39 (s, 1H), 4.19 (s, 3H), 1.82 (s, 3H), 1.78 (s, 3H).

### Example 6, Method AF

### Preparation of N-(4-((3-(5-(dimethylphosphoryl)-1-methyl-1H-pyrazol-3-yl)-2-methoxyphenyl)amino)-5-propionylpyridin-2-yl)cyclopropanecarboxamide (B2)

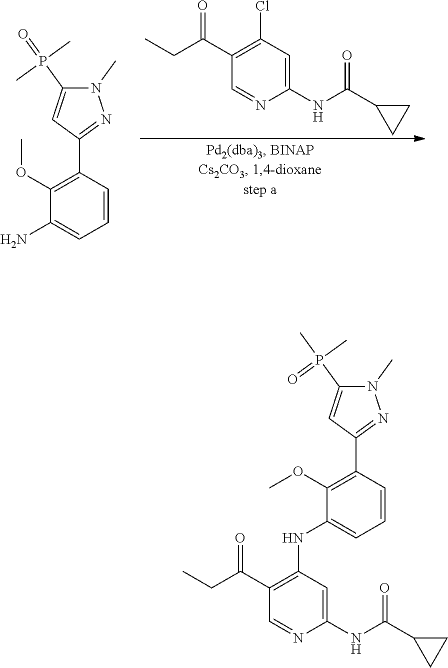

Step a. N-(4-((3-(5-(Dimethylphosphoryl)-1-methyl-1H-pyrazol-3-yl)-2-methoxyphenyl)amino)-5-propionylpyridin-2-yl)cyclopropanecarboxamide (B2): To a solution of (3-(3-amino-2-methoxyphenyl)-1-methyl-1H-pyrazol-5-yl)dimethylphosphine oxide (50 mg, 0.18 mmol) in 1,4-dioxane (4 mL) was added N-(4-chloro-5-propionylpyridin-2-yl)cyclopropanecarboxamide (46 mg, 0.18 mmol), Pd₂(dba)₃ (18 mg, 0.02 mmol), BINAP (12 mg, 0.02 mmol) and Cs₂CO₃ (117 mg, 0.36 mmol). The mixture was stirred at 130° C. under N₂ atmosphere in a microwave apparatus for 2 hours. The solvent was concentrated and the residue was purified by silica gel chromatography column (DCM/MeOH=100/3) to give the final compound (10 mg, 11%) as a yellow solid.

### Example 7, Method AG

### Preparation of (4-bromophenyl)dimethylphosphine oxide

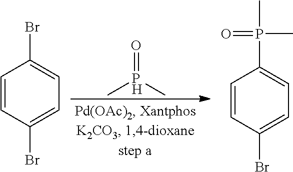

Step a. (4-Bromophenyl)dimethylphosphine oxide: To a solution of 1,4-dibromobenzene (585 mg, 2.5 mmol) in 1,4-dioxane (10 mL) was added dimethylphosphine oxide (234 mg, 3.0 mmol), K₂CO₃ (518 mg, 3.8 mmol), Pd(OAc)₂ (56 mg, 0.25 mmol) and Xantphos (115 mg, 0.20 mmol). The mixture was stirred at 125° C. under N₂ atmosphere in a microwave apparatus for 2 hours. The solvent was concentrated and the residue was purified by silica gel chromatography column (DCM/MeOH=100/3) to give the final compound (120 mg, 21%) as a yellow solid. ¹H NMR (400 MHZ, CDCl₃): δ 7.67-7.63 (m, 2H), 7.63-7.59 (m, 2H), 1.74 (s, 3H), 1.71 (s, 3H). LC-MS: 233.0 [M+H]⁺.

### Example 8, Method AH

### Preparation of (5-(3-amino-2-methoxyphenyl)pyrazin-2-yl)dimethylphosphine oxide

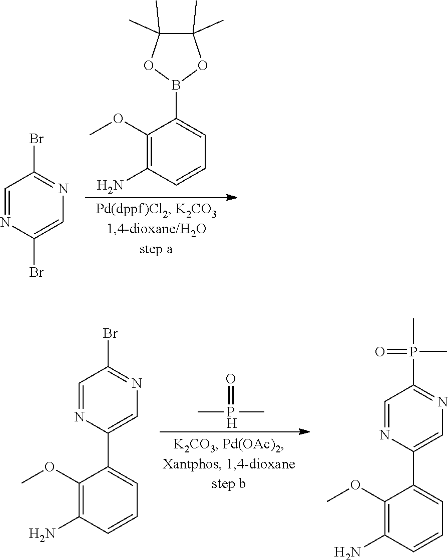

Step a. 3-(5-Bromopyrazin-2-yl)-2-methoxyaniline: To a solution of 2,5-dibromopyrazine (1.0 g, 4.2 mmol) in 1,4-dioxane/water (100 mL/5 mL) was added 2-methoxy-3-(4,4,5,5-tetramethyl-1,3,2-dioxaborolan-2-yl)aniline (1.1 g, 4.2 mmol), K₂CO₃ (1.2 g, 8.4 mmol) and Pd(dppf)Cl₂ (0.31 g, 0.4 mmol). The mixture was stirred at 90° C. under N₂ atmosphere overnight. The solvent was removed and the residue was purified by silica gel chromatography column (PE/EA=5/1) to give the final compound (470 mg, 40%) as a yellow solid. ¹H NMR (300 MHz, DMSO-d₆): δ 8.91 (s, 1H), 8.84 (s, 1H), 6.97 (t, J=7.5 Hz, 1H), 6.88 (d, J=7.8 Hz, 1H), 6.86-6.85 (m, 1H), 5.14 (s, 2H), 3.45 (s, 3H). LC-MS: 280.0 [M+H]⁺.

Step b. (5-(3-Amino-2-methoxyphenyl)pyrazin-2-yl)dimethylphosphine oxide: To a solution of 3-(5-bromopyrazin-2-yl)-2-methoxyaniline (200 mg, 0.7 mmol) in 1,4-dioxane (5 mL) was added dimethylphosphine oxide (84 mg, 1.1 mmol), K₂CO₃ (160 mg, 1.1 mmol), Pd(OAc)₂ (20 mg, 0.072 mmol) and Xantphos (42 mg, 0.072 mmol). The mixture was stirred at 125° C. under N₂ atmosphere in a microwave apparatus for 50 minutes. The solvent was concentrated and the residue was purified by silica gel chromatography column (DCM/MeOH=30/1) to give the final compound (194 mg, 97%) as a blue solid. ¹H NMR (300 MHz, DMSO-d₆): δ 8.91 (s, 1H), 8.84 (s, 1H), 6.97 (t, J=7.5 Hz, 1H), 6.88 (d, J=7.8 Hz, 1H), 6.86-6.85 (m, 1H), 5.14 (s, 2H), 3.45 (s, 3H), 1.80 (s, 3H), 1.75 (s, 3H). LC-MS: 278.1 [M+H]⁺.

### Example 9, Method AI

### Preparation of N-(4-((3-(5-(dicyclopropylphosphoryl)-1-methyl-1H-pyrazol-3-yl)-2-methoxyphenyl)amino)-5-propionylpyridin-2-yl)cyclopropanecarboxamide (B37)

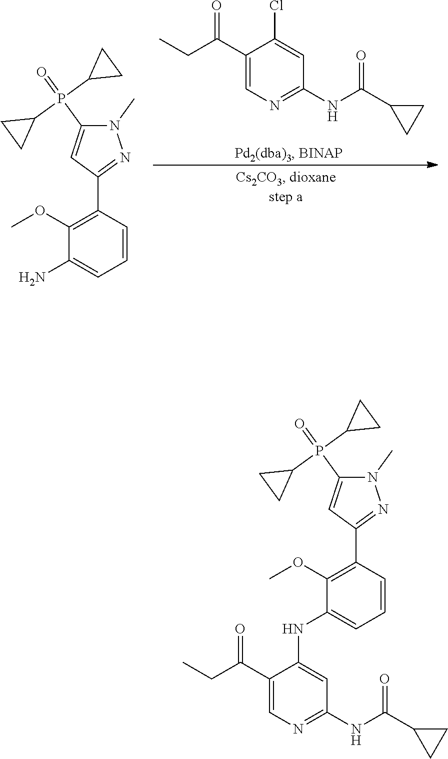 [SMILES: COc1c(N)cccc1-c1cc(P(=O)(C2CC2)C2CC2)n(C)n1 ; CCC(=O)c1cnc(NC(=O)C2CC2)cc1Cl ; "Pd2(dba)3, BINAP" ; CCC(=O)c1cnc(NC(=O)C2CC2)cc1Nc1cccc(-c2cc(P(=O)(C3CC3)C3CC3)n(C)n2)c1OC]

Step a. N-(4-((3-(5-(Dicyclopropylphosphoryl)-1-methyl-1H-pyrazol-3-yl)-2-methoxyphenyl)amino)-5-propionylpyridin-2-yl)cyclopropanecarboxamide (B37): To a solution of (3-(3-amino-2-methoxyphenyl)-1-methyl-1H-pyrazol-5-yl)dicyclopropylphosphine oxide (2.7 g, 8.15 mmol) and N-(4-chloro-5-propionylpyridin-2-yl)cyclopropanecarboxamide (2.68 g, 10.60 mmol) in 1,4-dioxane (30 mL) was added Cs₂CO₃ (5.31 g, 16.30 mmol), Pd₂(dba)₃ (0.37 g, 0.41 mmol) and BINAP (0.24 g, 0.41 mmol). The mixture was stirred at 130° C. under N₂ atmosphere for 2 hours. The solvent was concentrated and the residue was purified by silica gel chromatography column (DCM/MeOH=40/1) to give the crude product, which was rinsed by ethanol (20 mL) to give the final compound (1.56 g, 35%) as a yellow solid.

### Example 10, Method AJ

### Preparation of (3-bromo-1-cyclopropyl-1H-pyrazol-5-yl)dimethylphosphine oxide

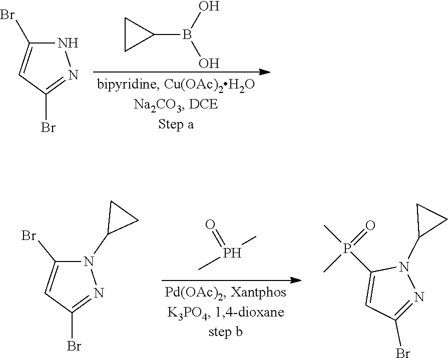

Step a. 3,5-Dibromo-1-cyclopropyl-1H-pyrazole: To a solution of 3,5-dibromo-1H-pyrazole (1.7 g, 7.6 mmol) in DCE (30 mL) was added cyclopropylboronic acid (1.3 g, 15.1 mmol), Na₂CO₃ (1.6 g, 15.1 mmol), bipyridine (1.2 g, 7.6 mmol) and CuOAc·H₂O (1.6 g, 7.6 mmol). The mixture was stirred at 75° C. overnight. Saturated NH₄Cl aqueous solution (30 mL) was added and the aqueous layer was extracted by dichloromethane (20 mL×3). The organic layers were combined and washed by saturated NH₄Cl aqueous solution (20 mL), dried by Na₂SO₄ and concentrated. The residue was purified by silica gel chromatography column (PE/EA=20/1) to give the final compound (194 mg, 97%) as a yellow oil. ¹H NMR (300 MHZ, CDCl₃): δ 6.29 (s, 1H), 3.50-3.36 (m, 1H), 1.27-1.17 (m, 2H), 1.12-1.03 (m, 2H). LC-MS: 264.8 [M+H]⁺.

Step b. (3-Bromo-1-cyclopropyl-1H-pyrazol-5-yl)dimethylphosphine oxide: To a solution of 3,5-dibromo-1-cyclopropyl-1H-pyrazole (756 mg, 2.8 mmol) in 1,4-dioxane (5 mL) was added dimethylphosphine oxide (334 mg, 4.3 mmol), K₃PO₄ (720 mg, 3.4 mmol), Pd(OAc)₂ (51 mg, 0.23 mmol) and Xantphos (133 mg, 0.23 mmol). The mixture was stirred at 125° C. under N₂ atmosphere in a microwave apparatus for 50 minutes. The solvent was concentrated and the residue was purified by silica gel chromatography column (DCM/MeOH=75/1) to give the final compound (175 mg, 23%) as a yellow oil. ¹H NMR (300 MHZ, CDCl₃): δ 6.43 (s, 1H), 4.31-4.13 (m, 1H), 1.88 (s, 3H), 1.83 (s, 3H), 1.43-1.34 (m, 2H), 1.15-1.01 (m, 2H). LC-MS: 263.0 [M+H]⁺.

### Example 11, Method AK

### Preparation of N-(5-((3-(5-(dimethylphosphoryl)-1-methyl-1H-pyrazol-3-yl)-2-methoxyphenyl)amino)-6-propionylpyridazin-3-yl)cyclopropanecarboxamide (B18)

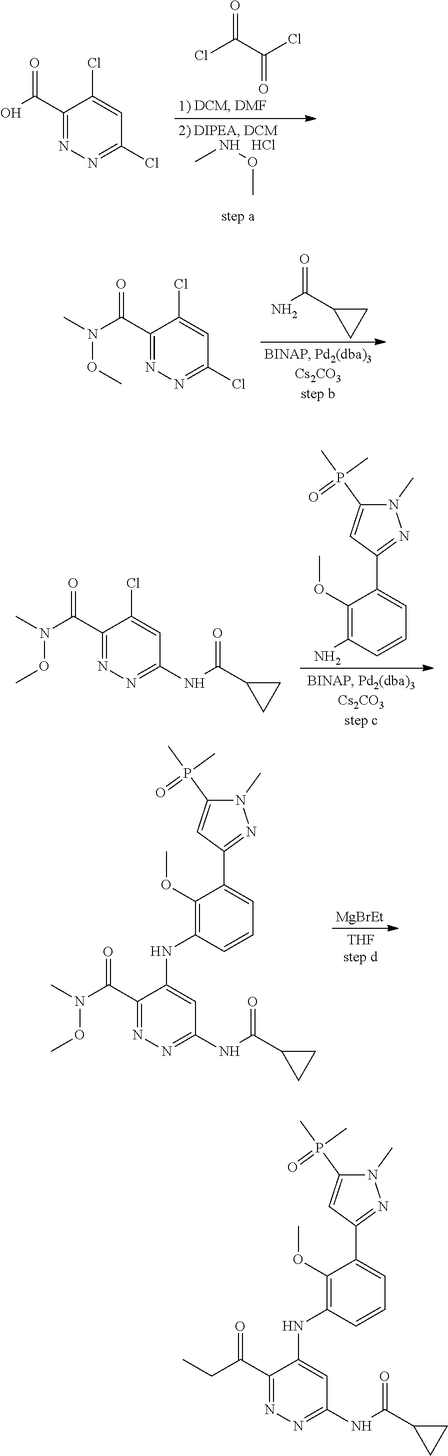

Step a. 4,6-Dichloro-N-methoxy-N-methylpyridazine-3-carboxamide: To a solution of 4,6-dichloropyridazine-3-carboxylic acid in dichloromethane (100 mL) was added oxalyl dichloride (5.2 mL, 60.2 mmol) and DMF (one drop) at 0° C. The mixture was stirred at room temperature for 5 hours. The solvent was removed and the residue was dissolved in dichloromethane (10 mL). N,O-dimethylhydroxylamine hydrochloride (4.4 g, 45.3 mmol) and DIPEA (10.5 mL, 60.4 mmol) dissolved in dichloromethane (100 mL) was added to the solution. The mixture was stirred at room temperature for 5 minutes. The solvent was removed and the residue was purified by silica gel chromatography column (DCM) to give the final compound (3.2 g, 45%) as a white solid. ¹H NMR (300 MHZ, CDCl₃): δ 7.64 (s, 1H), 3.60 (s, 3H), 3.41 (s, 3H). LC-MS: 236.0 [M+H]⁺.

Step b. 4-Chloro-6-(cyclopropanecarboxamido)-N-methoxy-N-methylpyridazine-3-carboxamide: To a solution of 4,6-dichloro-N-methoxy-N-methylpyridazine-3-carboxamide in 1,4-dioxane (3 mL) was added cyclopropanecarboxamide (260 mg, 3.1 mmol), Cs₂CO₃ (2.0 g, 6.1 mmol), Pd₂(dba)₃ (140 mg, 0.3 mmol) and BINAP (95 mg, 0.3 mmol). The mixture was stirred at 115° C. for 2 hours. The solvent was concentrated and the residue was purified by silica gel chromatography column (PE/EA=5/1) to give the final compound (320 mg, 37%) as a yellow oil. ¹H NMR (400 MHZ, DMSO-d₆): δ 11.84 (s, 1H), 8.54 (s, 1H), 3.52 (s, 3H), 3.35 (s, 3H), 2.11-2.03 (m, 1H), 0.96-0.90 (m, 2H), 0.91-0.89 (m, 2H). LC-MS: 285.1 [M+H]⁺.

Step c. 6-(Cyclopropanecarboxamido)-4-((3-(5-(dimethylphosphoryl)-1-methyl-1H-pyrazol-3-yl)-2-methoxyphenyl)amino)-N-methoxy-N-methylpyridazine-3-carboxamide: To a solution of 4-chloro-6-(cyclopropanecarboxamido)-N-methoxy-N-methylpyridazine-3-carboxamide (129 mg, 0.5 mmol) in 1,4-dioxane (3 mL) was added (3-(3-amino-2-methoxyphenyl)-1-methyl-1H-pyrazol-5-yl)dimethylphosphine oxide (127 mg, 0.5 mmol), Cs₂CO₃ (296 mg, 0.9 mmol), Pd₂(dba)₃ (42.3 mg, 0.05 mmol) and BINAP (29 mg, 0.05 mmol). The mixture was stirred at 130° C. under N₂ atmosphere for 2 hours. The solvent was concentrated and the residue was purified by silica gel chromatography column (DCM/MeOH=20/1) to give the final compound (105 mg, 44%) as a yellow solid. LC-MS: 528.2 [M+H]⁺.

Step d. N-(5-((3-(5-(Dimethylphosphoryl)-1-methyl-1H-pyrazol-3-yl)-2-methoxyphenyl)amino)-6-propionylpyridazin-3-yl)cyclopropanecarboxamide (B18): To a solution of 6-(cyclopropanecarboxamido)-4-((3-(5-(dimethylphosphoryl)-1-methyl-1H-pyrazol-3-yl)-2-methoxyphenyl)amino)-N-methoxy-N-methylpyridazine-3-carboxamide (165 mg, 0.31 mmol) in dry tetrahydrofuran (10 mL) was added ethylmagnesium bromide (1.25 mL, 1.25 mmol) under N₂ atmosphere at 0° C. The mixture was stirred at room temperature for an hour. Saturated NH₄Cl aqueous solution (10 mL) was added to quench the solution. The aqueous layer was extracted by ethyl acetate (50 mL×3). The organic layers were combined, dried by Na₂SO₄ and concentrated. The residue was purified by silica gel chromatography column (DCM/MeOH=20/1) to give the crude product, which was rinsed by EA/Et₂O (4 mL/4 mL) to give the desired product as a yellow oil (7 mg, 4.5%).

### Example 12, Method AL

### Preparation of 6-(cyclopropanecarboxamido)-4-((3-(5-(dicyclopropylphosphoryl)-1-methyl-1H-pyrazol-3-yl)-2-methoxyphenyl)amino)-N-(methyl-d₃)pyridazine-3-carboxamide (B41)

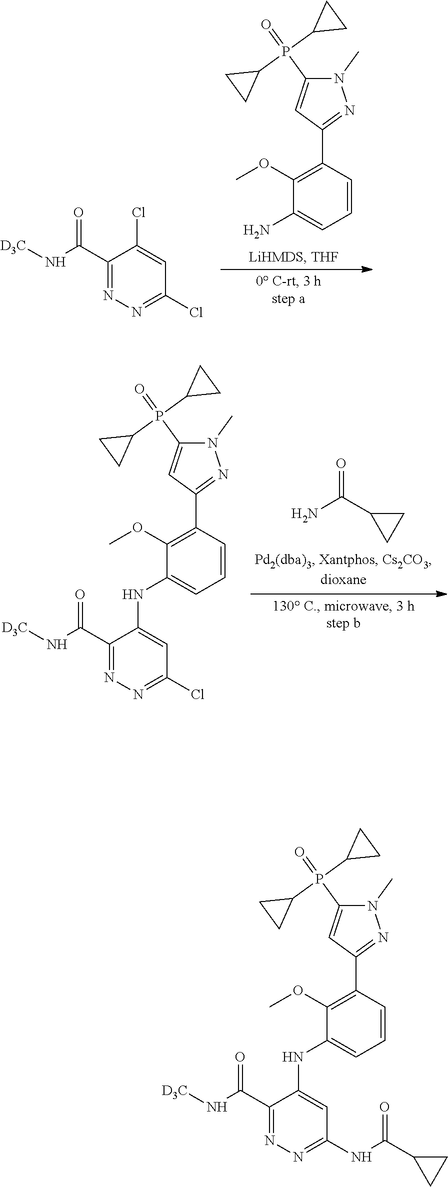 [SMILES: [2H]C([2H])([2H])NC(=O)c1nnc(Cl)cc1Cl ; COc1c(N)cccc1-c1cc(P(=O)(C2CC2)C2CC2)n(C)n1 ; [2H]C([2H])([2H])NC(=O)c1nnc(Cl)cc1Nc1cccc(-c2cc(P(=O)(C3CC3)C3CC3)n(C)n2)c1OC ; NC(=O)C1CC1 ; [2H]C([2H])([2H])NC(=O)c1nnc(NC(=O)C2CC2)cc1Nc1cccc(-c2cc(P(=O)(C3CC3)C3CC3)n(C)n2)c1OC]

Step a. 6-Chloro-4-((3-(5-(dicyclopropylphosphoryl)-1-methyl-1H-pyrazol-3-yl)-2-methoxyphenyl)amino)-N-(methyl-d₃)pyridazine-3-carboxamide: To a solution of 4,6-dichloro-N-(methyl-d₃)pyridazine-3-carboxamide (3.1 g, 14.9 mmol) and (3-(3-amino-2-methoxyphenyl)-1-methyl-1H-pyrazol-5-yl)dicyclopropylphosphine oxide (3.8 g, 11.5 mmol) in dry tetrahydrofuran (100 mL) was added IN LiHMDS (28.7 mL, 28.7 mmol) under N₂ atmosphere. The mixture was stirred at room temperature for 3 hours. Saturated NH₄Cl (100 mL) aqueous solution was added and the aqueous layer was extracted by ethyl acetate (100 mL×3). The combined organic layer was dried by Na₂SO₄ and concentrated. The residue was purified by silica gel chromatography column (DCM/MeOH=20/1) to give the final compound (3.0 g, 52%) as a yellow solid. ¹H NMR (300 MHz, DMSO-d₆) δ 11.19 (s, 1H), 9.39 (s, 1H), 7.75 (d, J=7.8 Hz, 1H), 7.53 (d, J=7.7 Hz, 1H), 7.35-7.20 (m, 3H), 4.13 (s, 3H), 3.63 (s, 3H), 1.42-1.26 (m, 2H), 0.97-0.87 (m, 4H), 0.87-0.73 (m, 4H). LC-MS: 503.7 [M+H]⁺.

Step b. 6-(Cyclopropanecarboxamido)-4-((3-(5-(dicyclopropylphosphoryl)-1-methyl-1H-pyrazol-3-yl)-2-methoxyphenyl)amino)-N-(methyl-d₃)pyridazine-3-carboxamide (B41): To a solution of 6-chloro-4-((3-(5-(dicyclopropylphosphoryl)-1-methyl-1H-pyrazol-3-yl)-2-methoxyphenyl)amino)-N-(methyl-d₃)pyridazine-3-carboxamide (3.0 g, 6.0 mmol) in 1,4-dioxane (25 mL) was added Pd₂(dba)₃ (550 mg, 0.6 mmol), Xantphos (346 mg, 0.6 mmol) and Cs₂CO₃ (3.9 g, 12.0 mmol). The mixture was stirred at 130° C. under N₂ atmosphere in a microwave apparatus for 3 hours. The solvent was filtered and the filtrate was concentrated. The residue was purified by silica gel chromatography column (DCM/MeOH=30/1) to give the crude product, which was rinsed by Et₂O to give the desired product (1.7 g, 51%) as a yellow solid.

### Example 13, Method AM

### Preparation of N-(4-((4-(5-(dimethylphosphoryl)-1-methyl-1H-pyrazol-3-yl)-3-methoxypyridin-2-yl)amino)-5-propionylpyridin-2-yl)cyclopropanecarboxamide (B19)

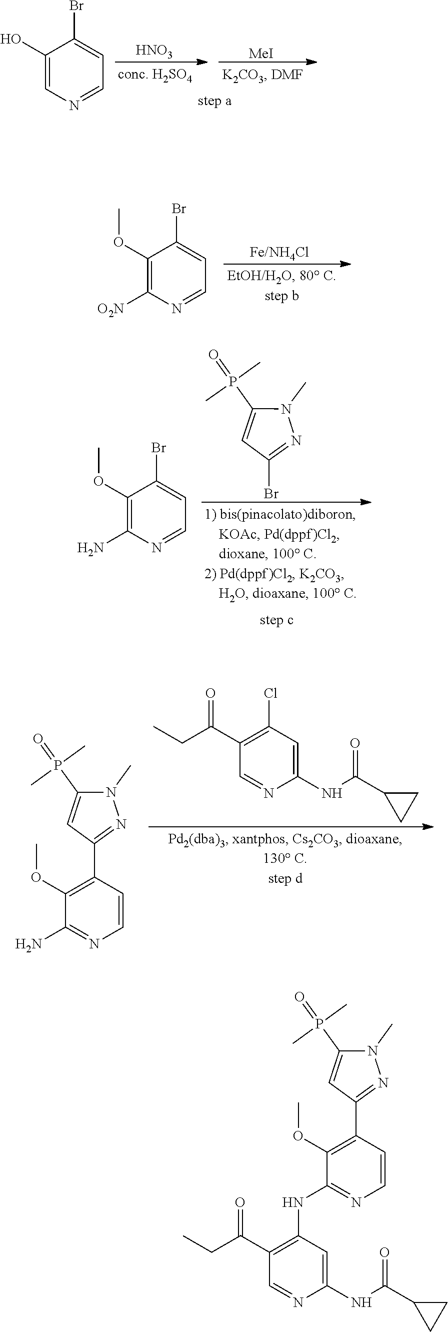

Step a. 4-Bromo-3-methoxy-2-nitropyridine: To a solution of 4-bromopyridin-3-ol (0.8 g, 4.6 mmol) in concentrated H₂SO₄ (5 mL) was added concentrated HNO₃ (0.4 mL) at 0° C. The mixture was stirred at this temperature for 0.5 hour. The solvent was poured into ice water. The aqueous layer was extracted by ethyl acetate (150 mL×2). The organic layers were combined, dried by Na₂SO₄ and concentrated. The residue was dissolved in DMF (5 mL). K₂CO₃ (0.5 g, 3.6 mmol) and iodomethane (0.5 mL) were added to the solution. The mixture was stirred at room temperature for an hour. The solvent was removed and the residue was purified by silica gel chromatography column (PE/EA=2/1) to give the final compound (260 mg, 24%) as a yellow solid. ¹H NMR (300 MHz, CDCl₃): δ 8.10 (d, J=5.1 Hz, 1H), 7.79 (d, J=4.8 Hz, 1H), 4.05 (s, 3H).

Step b. 4-Bromo-3-methoxypyridin-2-amine: To a solution of 4-bromo-3-methoxy-2-nitropyridine (260 mg, 1.2 mmol) in ethanol/water (15 mL/1 mL) was added iron powder (260 mg, 6.0 mmol) and NH₄Cl (340 mg, 6.0 mmol). The mixture was stirred at 80° C. for an hour. The solvent was filtered via diatomite and the filtrate was extracted by ethyl acetate (50 mL). The organic layer was washed by saturated NaHCO₃ aqueous solution (10 mL), dried by Na₂SO₄ and concentrated. The residue was purified by silica gel chromatography column (PE/EA=1/1) to give the final compound (185 mg, 82%) as a yellow solid. ¹H NMR (300 MHZ, CDCl₃): δ 7.62 (d, J=5.4 Hz, 1H), 6.82 (d, J=5.4 Hz, 1H), 4.76 (s, 2H), 3.85 (s, 3H).

Step c. (3-(2-Amino-3-methoxypyridin-4-yl)-1-methyl-1H-pyrazol-5-yl)dimethylphosphine oxide: To a solution of 4-bromo-3-methoxypyridin-2-amine (185 mg, 0.9 mmol) in 1,4-dioxane (15 mL) was added (Bpin)₂ (460 mg, 1.8 mmol), KOAc (230 mg, 2.3 mmol) and Pd(dppf)Cl₂ (66 mg, 0.09 mmol). The mixture was stirred at 100° C. overnight under N₂ atmosphere. (3-bromo-1-methyl-1H-pyrazol-5-yl)dimethylphosphine oxide (156 mg, 0.7 mmol), K₂CO₃ (230 mg, 1.7 mmol), Pd(dppf)Cl₂ (48 mg, 0.07 mmol) and water (1 mL) was added to the solution. The mixture was stirred at 100° C. for 6 hours under N₂ atmosphere. The solvent was removed and the residue was purified by silica gel chromatography column (DCM/MeOH=50/1) to give the final compound (60 mg, 32%) as a light yellow solid. LC-MS: 281.1 [M+H]⁺.

Step d. N-(4-((4-(5-(Dimethylphosphoryl)-1-methyl-1H-pyrazol-3-yl)-3-methoxypyridin-2-yl)amino)-5-propionylpyridin-2-yl)cyclopropanecarboxamide (B19): To a solution of (3-(2-amino-3-methoxypyridin-4-yl)-1-methyl-1H-pyrazol-5-yl)dimethylphosphine oxide (60 mg, 0.21 mmol) and N-(4-chloro-5-propionylpyridin-2-yl)cyclopropanecarboxamide (63 mg, 0.25 mmol) in 1,4-dioxane (2 mL) was added Pd₂(dba)₃ (20 mg, 0.02 mmol), Xantphos (12 mg, 0.02 mmol) and Cs₂CO₃ (136 mg, 0.42 mmol). The mixture was stirred at 130° C. under N₂ atmosphere in a microwave apparatus for 2 hours. The solvent was concentrated and the residue was purified by silica gel chromatography column (DCM/MeOH=75/1) to give the crude product, which was rinsed by EA/Et₂O (2 mL/1 mL) to give the desired product (6 mg, 6%) as a yellow solid.

### Example 14, Method AN

### Preparation of N-(4-((6-(5-(dicyclopropylphosphoryl)-1-methyl-1H-pyrazol-3-yl)-5-methoxypyrimidin-4-yl)amino)-5-propionylpyridin-2-yl)cyclopropanecarboxamide (B46)

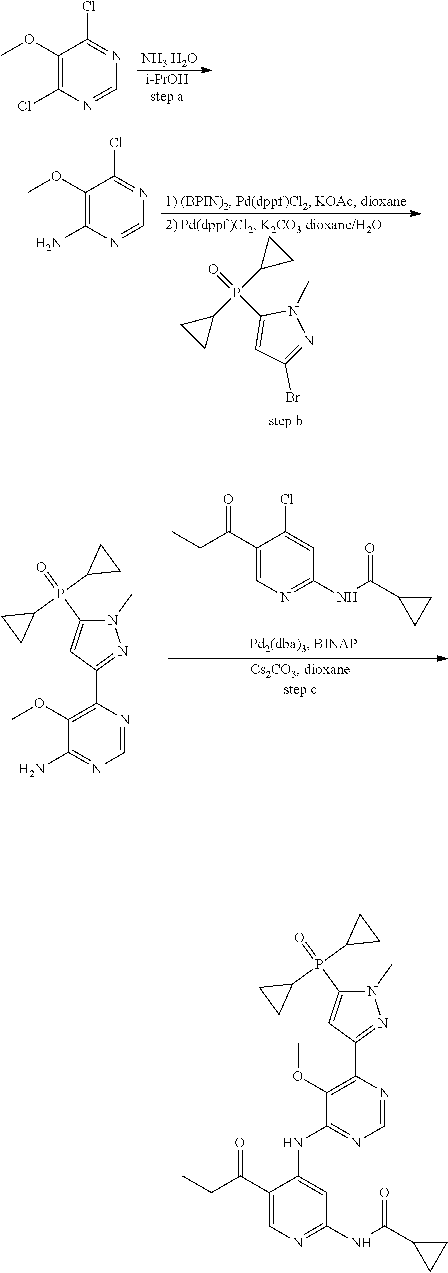

Step a. 6-Chloro-5-methoxypyrimidin-4-amine: To a solution of 4,6-dichloro-5-methoxypyrimidine (500 mg, 2.8 mmol) in i-PrOH (5 mL) was added 30% ammonia hydrate (5 mL). The mixture was stirred at 80° C. in a sealed tube overnight. The solvent was removed and the residue was rinsed by water, dried in vacuum to give the desired product (360 mg, 81%) as a white solid. ¹H NMR (300 MHz, CDCl₃): δ 8.12 (s, 1H), 5.36 (s, 2H), 3.89 (s, 3H).

Step b. (3-(6-Amino-5-methoxypyrimidin-4-yl)-1-methyl-1H-pyrazol-5-yl)dicyclopropylphosphine oxide: To a solution of (3-bromo-1-methyl-1H-pyrazol-5-yl)dicyclopropylphosphine oxide (500 mg, 1.7 mmol) in 1,4-dioxane (20 mL) was added (Bpin)₂ (635 mg, 1.5 mmol), KOAc (500 mg, 5.1 mmol), Pd(dppf)Cl₂ (124 mg, 0.17 mmol). The mixture was stirred at 100° C. under N₂ atmosphere overnight. Water (1 mL), K₂CO₃ (469 mg, 3.4 mmol), Pd(dppf)Cl₂ (124 mg, 0.17 mmol) and 6-chloro-5-methoxypyrimidin-4-amine (270 mg, 1.7 mmol) was added to the solution. The mixture was stirred at 100° C. for 10 hours. The solvent was removed and the residue was purified by silica gel chromatography column (DCM/MeOH/NH₃·H₂O=50/2/1) to give the desired product (530 mg, 93%) as a black solid. 1H NMR (300 MHz, CDCl₃): δ 8.38 (s, 1H), 7.31 (s, 1H), 5.34 (s, 2H), 4.29 (s, 3H), 3.78 (s, 3H), 1.13-0.83 (m, 10H).

Step c. N-(4-((6-(5-(Dicyclopropylphosphoryl)-1-methyl-1H-pyrazol-3-yl)-5-methoxypyrimidin-4-yl)amino)-5-propionylpyridin-2-yl)cyclopropanecarboxamide (B46): To a solution of (3-(6-amino-5-methoxypyrimidin-4-yl)-1-methyl-1H-pyrazol-5-yl)dicyclopropylphosphine oxide (50 mg, 0.15 mmol) and N-(4-chloro-5-propionylpyridin-2-yl)cyclopropanecarboxamide (45 mg, 0.18 mmol) in 1,4-dioxane (2 mL) was added BINAP (9 mg, 0.015 mmol), Pd₂(dba)₃ (13 mg, 0.015 mmol) and Cs₂CO₃ (97 mg, 0.3 mmol). The mixture was stirred at 130° C. under N₂ atmosphere for 2 hours in a microwave apparatus. The solvent was concentrated and the residue was purified by silica gel chromatography column (DCM/MeOH=30/1) to give the final compound (24 mg, 30%) as a white solid.

Table 1 shows a selection of the compounds prepared according to the methods described above and the method numbers are indicated in the third column of the table.

TABLE 1
Selected compounds (B1-B46)

| NO. | Structure | Method | ¹HNMR & LC-MS |
|---|---|---|---|
| B1 | 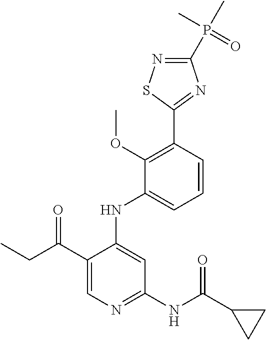 [SMILES: CCC(=O)c1cnc(NC(=O)C2CC2)cc1Nc1cccc(-c2nc(P(C)(C)=O)ns2)c1OC] | AA, AC, AD | ¹H NMR (300 MHz, DMSO-d₆): δ 10.99 (s, 1H), 10.95 (s, 1H), 8.91 (s, 1H), 8.19 (d, J = 7.8 Hz, 1H), 7.84 (s, 1H), 7.71 (d, J = 7.8 Hz, 1H), 7.46 (t, J = 8.1 Hz, 1H), 3.88 (s, 3H), 3.16 (q, J = 6.9 Hz, 2H), 2.05- 1.96 (m, 1H), 1.92 (s, 3H), 1.87 (s, 3H), 1.13 (t, J = 7.2 Hz, 3H), 0.81-0.68 (m, 4H). LC-MS: 500.1 [M + H]⁺. |
| B2 | 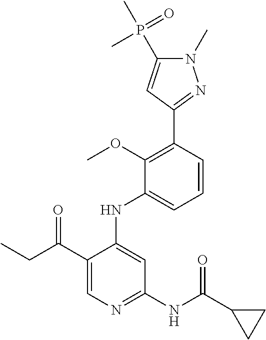 [SMILES: CCC(=O)c1cnc(NC(=O)C2CC2)cc1Nc1cccc(-c2cc(P(C)(C)=O)n(C)n2)c1OC] | AA, AC, AE, AF, AI | ¹H NMR (300 MHz, DMSO-d₆): δ 11.09 (s, 1H), 10.92 (s, 1H), 8.88 (s, 1H), 8.03 (s, 1H), 7.66 (d, J = 7.5 Hz, 1H), 7.43 (d, J = 7.5 Hz, 1H), 7.23 (t, J = 7.8 Hz, 1H), 7.08 (s, 1H), 4.16 (s, 3H), 3.59 (s, 3H), 3.13 (q, J = 7.5 Hz, 2H), 2.07-1.95 (m, 1H), 1.84 (s, 3H), 1.80 (s, 3H), 1.12 (t, J = 6.9 Hz, 3H), 0.84-0.71 (m, 4H). LC-MS: 496.2 [M + H]⁺. |
| B3 | 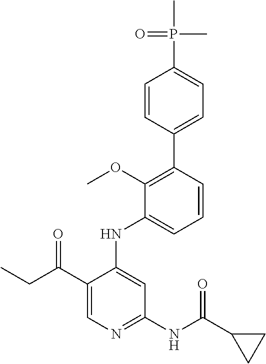 [SMILES: CCC(=O)c1cnc(NC(=O)C2CC2)cc1Nc1cccc(-c2ccc(P(C)(C)=O)cc2)c1OC] | AA, AC, AF, AG, AI | ¹H NMR (300 MHz, DMSO-d₆): δ 11.07 (s, 1H), 10.94 (s, 1H), 8.88 (s, 1H), 8.07 (s, 1H), 7.93-7.78 (m, 2H), 7.77-7.64 (m, 2H), 7.50 (d, J = 7.5 Hz, 1H), 7.36-7.15 (m, 2H), 3.36 (s, 3H), 3.19-3.06 (m, 2H), 2.09-1.96 (m, 1H), 1.72 (s, 3H), 1.67 (s, 3H), 1.15-1.08 (m, 3H), 0.85-0.72 (m, 4H). LC-MS: 492.2 [M + H]⁺. |
| B4 | 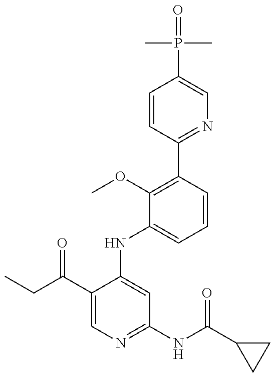 [SMILES: CCC(=O)c1cnc(NC(=O)C2CC2)cc1Nc1cccc(-c2ccc(P(C)(C)=O)cn2)c1OC] | AA, AC, AH, AI | ¹H NMR (300 MHz, DMSO-d₆): δ 11.06 (s, 1H), 10.93 (s, 1H), 9.03 (d, J = 6.9 Hz, 1H), 8.89 (s, 1H), 8.28-8.18 (m, 1H), 8.04 (s, 1H), 8.00 (d, J = 8.4 Hz, 1H), 7.58 (s, 1H), 7.55 (s, 1H), 7.37-7.27 (m, 1H), 3.49 (s, 3H), 3.19-3.09 (m, 2H), 2.05- 1.96 (m, 1H), 1.79 (s, 3H), 1.74 (s, 3H), 1.12 (t, J = 7.2 Hz, 3H), 0.83-0.77 (m, 4H). LC-MS: 493.2 [M + H]⁺. |
| B5 | 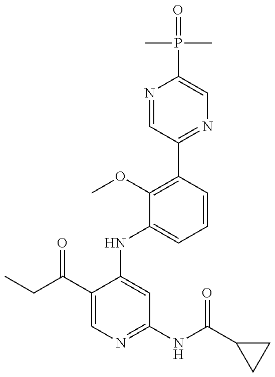 [SMILES: CCC(=O)c1cnc(NC(=O)C2CC2)cc1Nc1cccc(-c2cnc(P(C)(C)=O)cn2)c1OC] | AA, AC, AH, AI | ¹H NMR (300 MHz, DMSO-d₆): δ 11.03 (s, 1H), 10.93 (s, 1H), 9.24 (s, 1H), 9.18 (s, 1H), 8.89 (s, 1H), 8.01 (s, 1H), 7.66-7.58 (m, 2H), 7.38 (t, J = 7.8 Hz, 1H), 3.54 (s, 3H), 3.18-3.10 (m, 2H), 2.01 (s, 1H), 1.81 (s, 3H), 1.76 (s, 3H), 1.12 (t, J = 6.9 Hz, 3H), 0.83-0.73 (m, 4H). LC-MS: 494.1 [M + H]⁺. |
| B6 | 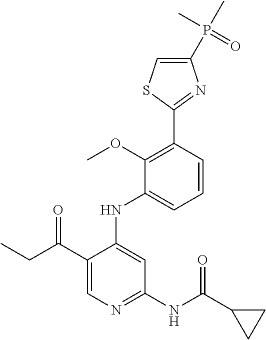 [SMILES: CCC(=O)c1cnc(NC(=O)C2CC2)cc1Nc1cccc(-c2nc(P(C)(C)=O)cs2)c1OC] | AA, AC, AH, AI | ¹H NMR (300 MHz, DMSO-d₆): δ 11.00 (s, 1H), 10.93 (s, 1H), 8.90 (s, 1H), 8.43 (d, J = 3.6 Hz, 1H), 8.12 (d, J = 7.2 Hz, 1H), 7.91 (s, 1H), 7.58 (d, J = 7.5 Hz, 1H), 7.35 (d, J = 8.1 Hz, 1H), 3.76 (s, 3H), 3.15 (q, J = 7.2 Hz, 2H), 2.05-1.95 (m, 1H), 1.78 (s, 3H), 1.74 (s, 3H), 1.13 (t, J = 7.2 Hz, 3H), 0.82-0.71 (m, 4H). LC-MS: 499.1 [M + H]⁺. |
| B7 | 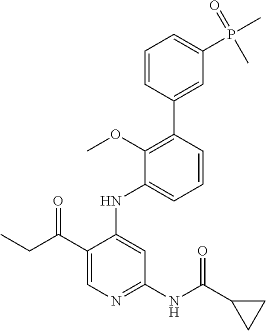 [SMILES: CCC(=O)c1cnc(NC(=O)C2CC2)cc1Nc1cccc(-c2cccc(P(C)(C)=O)c2)c1OC] | AA, AC, AF, AG, AI | ¹H NMR (300 MHz, DMSO-d₆): δ 11.06 (s, 1H), 10.91 (s, 1H), 8.87 (s, 1H), 8.06 (s, 1H), 7.93 (d, J = 11.7 Hz, 1H), 7.75 (d, J = 6.9 Hz, 2H), 7.62 (s, 1H), 7.49 (d, J = 7.8 Hz, 1H), 7.26 (dd, J = 13.5, 7.2 Hz, 2H), 3.34 (s, 3H), 3.12 (dd, J = 14.7, 7.5 Hz, 2H), 2.06-1.94 (m, 1H), 1.71 (s, 3H), 1.67 (s, 3H), 1.11 (t, J = 7.2 Hz, 3H), 0.84- 0.74 (m, 4H). LC-MS: 492.5 [M + H]⁺. |
| B8 | 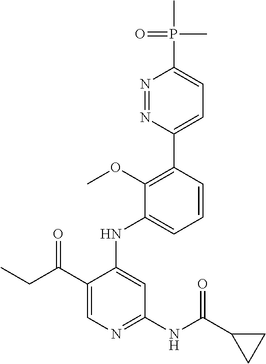 [SMILES: CCC(=O)c1cnc(NC(=O)C2CC2)cc1Nc1cccc(-c2ccc(P(C)(C)=O)nn2)c1OC] | AA, AC, AH, AI | ¹H NMR (300 MHz, DMSO-d₆): δ 11.06 (s, 1H), 10.95 (s, 1H), 8.90 (s, 1H), 8.29- 8.20 (m, 2H), 8.03 (s, 1H), 7.67-7.60 (m, 2H), 7.40 (t, J = 7.8 Hz, 1H), 3.49 (s, 3H), 3.18-3.09 (m, 2H), 2.05-1.98 (m, 1H), 1.88 (s, 3H), 1.84 (s, 3H), 1.12 (t, J = 6.9 Hz, 3H), 0.83-0.75 (m, 4H). LC-MS: 494.1 [M + H]⁺. |
| B9 | 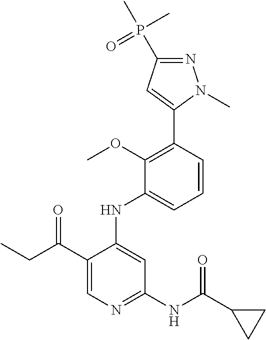 [SMILES: CCC(=O)c1cnc(NC(=O)C2CC2)cc1Nc1cccc(-c2cc(P(C)(C)=O)nn2C)c1OC] | AA, AC, AH, AI | ¹H NMR (300 MHz, DMSO-d₆): δ 11.01 (s, 1H), 10.93 (s, 1H), 8.88 (s, 1H), 7.97 (s, 1H), 7.58 (d, J = 7.5 Hz, 1H), 7.31 (t, J = 7.5 Hz, 1H), 7.20 (d, J = 6.9 Hz, 1H), 6.82 (s, 1H), 3.76 (s, 3H), 3.38 (s, 3H), 3.13 (q, J = 6.9 Hz, 2H), 2.06-1.95 (m, 1H), 1.71 (s, 3H), 1.66 (s, 3H), 1.11 (t, J = 6.9 Hz, 3H), 0.83-0.73 (m, 4H). LC-MS: 496.2 [M + H]⁺. |
| B10 | 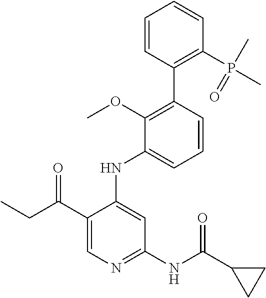 [SMILES: CCC(=O)c1cnc(NC(=O)C2CC2)cc1Nc1cccc(-c2ccccc2P(C)(C)=O)c1OC] | AA, AC, AF, AG, AI | ¹H NMR (300 MHz, DMSO-d₆): δ 10.99 (s, 1H), 10.93 (s, 1H), 8.87 (s, 1H), 8.04 (s, 1H), 7.98 (s, 1H), 7.58 (t, J = 6.3 Hz, 2H), 7.53 (d, J = 7.5 Hz, 1H), 7.39 (s, 1H), 7.23 (t, J = 7.8 Hz, 1H), 7.11 (d, J = 7.2 Hz, 1H), 3.37 (s, 3H), 3.11 (q, J = 6.9 Hz, 2H), 2.08-1.97 (m, 1H), 1.42 (d, J = 13.2 Hz, 3H), 1.28 (d, J = 13.2 Hz, 3H), 1.10 (t, J = 6.9 Hz, 3H), 0.89-0.72 (m, 4H). LC-MS: 492.1 [M + H]⁺. |
| B11 | 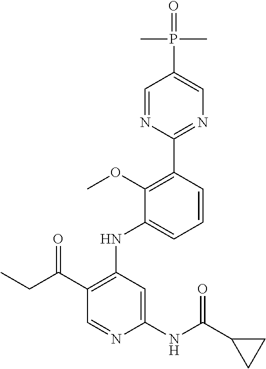 [SMILES: CCC(=O)c1cnc(NC(=O)C2CC2)cc1Nc1cccc(-c2ncc(P(C)(C)=O)cn2)c1OC] | AA, AC, AH, AI | ¹H NMR (300 MHz, DMSO-d₆): δ 11.02 (s, 1H), 10.93 (s, 1H), 9.25 (s, 1H), 9.23 (s, 1H), 8.89 (s, 1H), 8.03 (s, 1H), 7.68-7.57 (m, 2H), 7.34 (t, J = 8.0 Hz, 1H), 3.70 (s, 3H), 3.13 (q, J = 6.8 Hz, 2H), 2.05-1.99 (m, 1H), 1.86 (s, 3H), 1.82 (s, 3H), 1.17- 1.09 (m, 3H), 0.85-0.74 (m, 4H). LC-MS: 494.2 [M + H]⁺. |
| B12 | 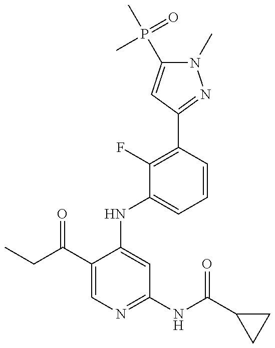 [SMILES: CCC(=O)c1cnc(NC(=O)C2CC2)cc1Nc1cccc(-c2cc(P(C)(C)=O)n(C)n2)c1F] | AA, AC, AE, AF, AI | ¹H NMR (300 MHz, DMSO-d₆): δ 10.91 (s, 1H), 10.88 (s, 1H), 8.87 (s, 1H), 7.84- 7.72 (m, 2H), 7.52-7.41 (m, 1H), 7.37- 7.27 (m, 1H), 7.06-6.98 (m, 1H), 4.17 (s, 3H), 3.18-3.05 (m, 2H), 2.04-1.92 (m, 1H), 1.83 (s, 3H), 1.78 (s, 3H), 1.11 (t, J = 7.2 Hz, 3H), 0.80-0.68 (m, 4H). LC-MS: 484.2 [M + H]⁺. |
| B13 | 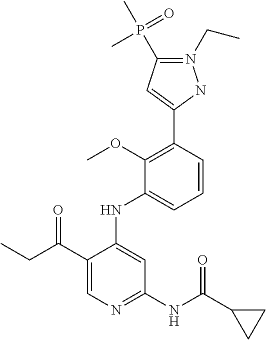 | AA, AC, AE, AF, AI | ¹H NMR (300 MHz, DMSO-d₆): δ 11.09 (s, 1H), 10.94 (s, 1H), 8.87 (s, 1H), 7.97 (s, 1H), 7.68 (d, J = 7.5 Hz, 1H), 7.43 (d, J = 7.2 Hz, 1H), 7.25 (d, J = 7.8 Hz, 1H), 7.06 (s, 1H), 4.55 (q, J = 6.9 Hz, 2H), 3.60 (s, 3H), 3.18-3.07 (m, 2H), 2.03-1.95 (m, 1H), 1.84 (s, 3H), 1.79 (s, 3H), 1.45 (t, J = 7.2 Hz, 3H), 1.13 (d, J = 7.2 Hz, 3H), 0.84- 0.75 (m, 1H). LC-MS: 510.2 [M + H]⁺. |
| B14 | 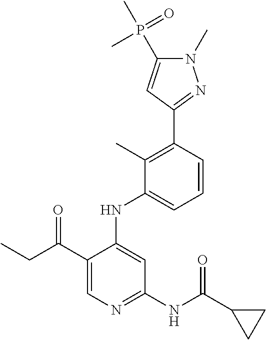 [SMILES: CCC(=O)c1cnc(NC(=O)C2CC2)cc1Nc1cccc(-c2cc(P(C)(C)=O)n(C)n2)c1C] | AA, AC, AE, AF, AI | ¹H NMR (300 MHz, DMSO-d₆): δ 10.81 (s, 1H), 10.74 (s, 1H), 8.83 (s, 1H), 7.49 (s, 1H), 7.45-7.37 (m, 1H), 7.37-7.23 (m, 2H), 6.90 (s, 1H), 4.15 (s, 3H), 3.12 (q, J = 7.5 Hz, 2H), 2.28 (s, 3H), 2.06-1.92 (m, 1H), 1.83 (s, 3H), 1.79 (s, 3H), 1.12 (t, J = 7.2 Hz, 3H), 0.91-0.56 (m, 4H). LC-MS: 480.2 [M + H]⁺. |
| B15 | 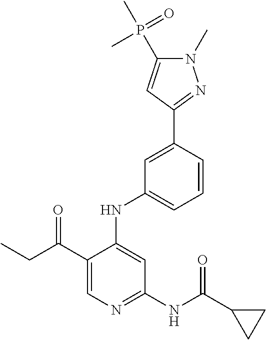 [SMILES: CCC(=O)c1cnc(NC(=O)C2CC2)cc1Nc1cccc(-c2cc(P(C)(C)=O)n(C)n2)c1] | AA, AC, AE, AF, AI | ¹H NMR (300 MHz, DMSO-d₆): δ 10.96 (s, 1H), 10.88 (s, 1H), 8.89-8.82 (m, 1H), 7.95 (s, 1H), 7.70-7.62 (m, 2H), 7.47 (t, J = 7.8 Hz, 1H), 7.28-7.18 (m, 2H), 4.13 (s, 3H), 3.11 (q, J = 7.2 Hz, 2H), 2.04- 1.94 (m, 1H), 1.82 (s, 3H), 1.78 (s, 3H), 1.12 (t, J = 7.5 Hz, 3H), 0.81-0.71 (m, 4H). LC-MS: 466.2 [M + H]⁺. |
| B16 | 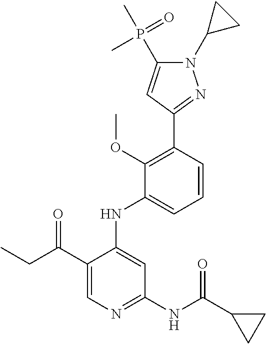 [SMILES: CCC(=O)c1cnc(NC(=O)C2CC2)cc1Nc1cccc(-c2cc(P(C)(C)=O)n(C3CC3)n2)c1OC] | AA, AC, AF, AI, AJ | ¹H NMR (300 MHz, DMSO-d₆): δ 11.07 (s, 1H), 10.99-10.91 (m, 1H), 8.87 (s, 1H), 7.94 (s, 1H), 7.63 (d, J = 7.5 Hz, 1H), 7.43 (d, J = 7.5 Hz, 1H), 7.23 (t, J = 7.8 Hz, 1H), 7.08 (s, 1H), 4.31 (s, 1H), 3.59 (s, 3H), 3.13 (q, J = 7.2 Hz, 2H), 2.08-1.93 (m, 1H), 1.89 (s, 3H), 1.84 (s, 3H), 1.37- 1.27 (m, 2H), 1.18-1.02 (m, 5H), 0.86- 0.69 (m, 4H). LC-MS: 522.2 [M + H]⁺. |
| B17 | 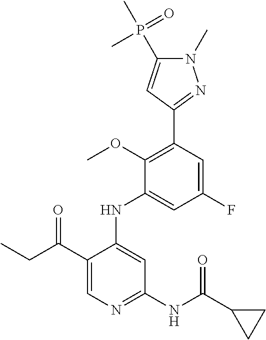 [SMILES: CCC(=O)c1cnc(NC(=O)C2CC2)cc1Nc1cc(F)cc(-c2cc(P(C)(C)=O)n(C)n2)c1OC] | AA, AC, AE, AF, AI | ¹H NMR (300 MHz, DMSO-d₆): δ 11.23 (s, 1H), 11.00 (s, 1H), 8.91 (s, 1H), 8.12 (s, 1H), 7.46-7.25 (m, 2H), 7.15 (s, 1H), 4.17 (s, 3H), 3.61 (s, 3H), 3.20-3.06 (m, 2H), 2.08-1.95 (m, 1H), 1.85 (s, 3H), 1.80 (s, 3H), 1.11 (t, J = 7.2 Hz, 3H), 0.86- 0.76 (m, 4H). LC-MS: 514.2 [M + H]⁺. |
| B18 | 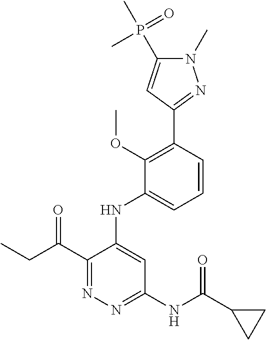 [SMILES: CCC(=O)c1nnc(NC(=O)C2CC2)cc1Nc1cccc(-c2cc(P(C)(C)=O)n(C)n2)c1OC] | AB, AC, AE, AF, AK | ¹H NMR (300 MHz, DMSO-d₆): δ 11.49 (s, 1H), 10.57 (s, 1H), 8.10 (s, 1H), 7.71 (d, J = 9.0 Hz, 1H), 7.42 (d, J = 7.5 Hz, 1H), 7.26 (t, J = 6.9 Hz, 1H), 7.08 (s, 1H), 4.16 (s, 3H), 3.60 (s, 3H), 3.32-3.31 (m, 2H), 2.13-2.04 (m, 1H), 1.84 (s, 3H), 1.80 (s, 3H), 1.16 (t, J = 7.5 Hz, 3H), 0.85- 0.84 (m, 2H), 0.82-0.78 (m, 2H). LC-MS: 497.2 [M + H]⁺. |
| B19 | 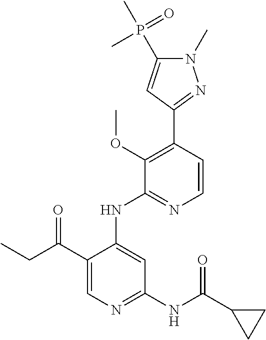 [SMILES: CCC(=O)c1cnc(NC(=O)C2CC2)cc1Nc1nccc(-c2cc(P(C)(C)=O)n(C)n2)c1OC] | AA, AE, AM | ¹H NMR (300 MHz, DMSO-d₆): δ 12.35 (s, 1H), 10.92 (s, 1H), 9.68 (s, 1H), 8.95 (s, 1H), 8.10 (d, J = 6.3 Hz, 1H), 7.45 (d, J = 5.1 Hz, 1H), 7.26 (s, 1H), 4.20 (s, 3H), 3.78 (s, 3H), 3.22-3.13 (m, 2H), 2.12- 2.03 (m, 1H), 1.85 (d, J = 13.8 Hz, 6H), 1.13 (t, J = 7.2 Hz, 3H), 0.90-0.81 (m, 4H). LC-MS: 497.2 [M + H]⁺. |
| B20 | 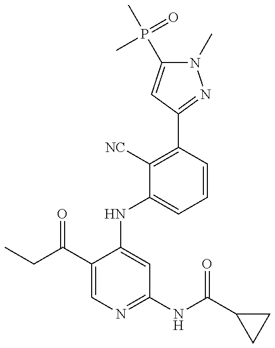 [SMILES: CCC(=O)c1cnc(NC(=O)C2CC2)cc1Nc1cccc(-c2cc(P(C)(C)=O)n(C)n2)c1C#N] | AA, AC, AE, AF, AI | ¹H NMR (300 MHz, DMSO-d₆): δ 11.23 (s, 1H), 11.00 (s, 1H), 8.93 (s, 1H), 7.86 (s, 1H), 7.83-7.73 (m, 1H), 7.69 (d, J = 9.3 Hz, 1H), 7.61 (d, J = 8.1, 1H), 7.23 (s, 1H), 4.18 (s, 3H), 3.22-3.08 (m, 2H), 2.05-1.94 (m, 1H), 1.83 (d, J = 14.1, 6H), 1.12 (t, J = 7.3 Hz, 3H), 0.82-0.72 (m, 4H). LC-MS: 491.2 [M + H]⁺. |
| B21 | 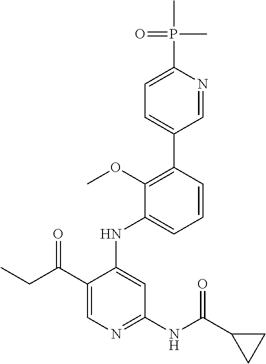 [SMILES: CCC(=O)c1cnc(NC(=O)C2CC2)cc1Nc1cccc(-c2ccc(P(C)(C)=O)nc2)c1OC] | AA, AC, AF, AG, AI | ¹H NMR (300 MHz, DMSO-d₆): δ 11.05 (s, 1H), 10.93 (s, 1H), 8.95 (s, 1H), 8.88 (s, 1H), 8.17 (s, 1H), 8.12-7.93 (m, 2H), 7.55 (s, 1H), 7.38-7.21 (m, 2H), 3.38 (s, 3H), 3.19-3.05 (m, 2H), 2.10-1.93 (m, 1H), 1.71 (d, J = 13.5 Hz, 6H), 1.19-1.02 (m, 3H), 0.88-0.70 (m, 4H). LC-MS: 493.2 [M + H]⁺. |
| B22 | 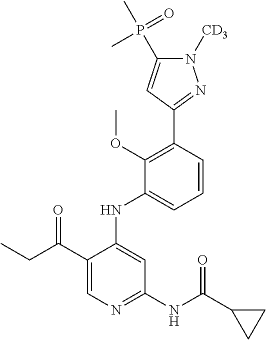 [SMILES: [2H]C([2H])([2H])n1nc(-c2cccc(Nc3cc(NC(=O)C4CC4)ncc3C(=O)CC)c2OC)cc1P(C)(C)=O] | AA, AC, AE, AF, AI | ¹H NMR (300 MHz, DMSO-d₆): δ 11.09 (s, 1H), 10.95 (s, 1H), 8.87 (s, 1H), 7.96 (s, 1H), 7.67 (d, J = 7.9 Hz, 1H), 7.43 (d, J = 7.8 Hz, 1H), 7.29-7.20 (m, 1H), 7.08 (s, 1H), 3.59 (s, 3H), 3.18-3.07 (m, 2H), 2.03-1.93 (m, 1H), 1.82 (d, J = 13.8 Hz, 6H), 1.12 (t, J = 6.9 Hz, 3H), 0.90-0.79 (m, 4H). LC-MS: 499.2 [M + H]⁺. |
| B23 | 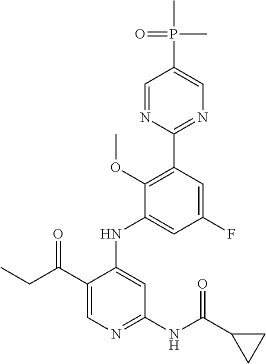 [SMILES: CCC(=O)c1cnc(NC(=O)C2CC2)cc1Nc1cc(F)cc(-c2ncc(P(C)(C)=O)cn2)c1OC] | AA, AC, AH, AI | ¹H NMR (300 MHz, DMSO-d₆): δ 11.16 (s, 1H), 11.03 (s, 1H), 9.27 (s, 1H), 9.25 (s, 1H), 8.92 (s, 1H), 8.09 (s, 1H), 7.59-7.50 (m, 1H), 7.43-7.33 (m, 1H), 3.71 (s, 3H), 3.19-3.03 (m, 2H), 2.09-1.95 (m, 1H), 1.84 (d, J = 13.8 Hz, 6H), 1.11 (t, J = 7.2 Hz, 3H), 0.90-0.79 (m, 4H). LC-MS: 512.2 [M + H]⁺. |
| B24 | 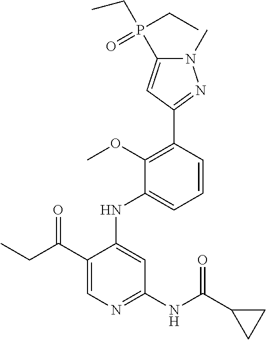 [SMILES: CCC(=O)c1cnc(NC(=O)C2CC2)cc1Nc1cccc(-c2cc(P(=O)(CC)CC)n(C)n2)c1OC] | AA, AC, AE, AF, AI | ¹H NMR (300 MHz, CDCl₃): δ 11.30 (s, 1H), 8.71 (s, 1H), 8.54 (s, 1H), 8.12 (s, 1H), 7.69 (d, J = 7.5 Hz, 1H), 7.50 (d, J = 7.8 Hz, 1H), 7.28-7.20 (m, 1H), 6.98 (s, 1H), 4.29 (s, 3H), 3.62 (s, 3H), 3.04 (q, J = 7.2 Hz, 2H), 2.08-1.94 (m, 4H), 1.64- 1.51 (m, 1H), 1.31-1.21 (m, 6H), 1.20- 1.14 (m, 3H), 1.13-1.03 (m, 2H), 0.95- 0.83 (m, 2H). LC-MS: 524.5 [M + H]⁺. |
| B25 | 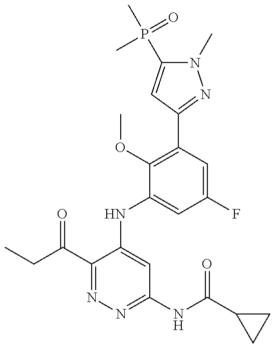 [SMILES: CCC(=O)c1nnc(NC(=O)C2CC2)cc1Nc1cc(F)cc(-c2cc(P(C)(C)=O)n(C)n2)c1OC] | AB, AC, AE, AF, AK | ¹H NMR (300 MHz, DMSO-d₆): δ 11.55 (s, 1H), 10.68 (s, 1H), 8.18 (s, 1H), 7.48- 7.42 (m, 1H), 7.37-7.30 (m, 1H), 7.13 (s, 1H), 4.17 (s, 3H), 3.61 (s, 3H), 3.53-3.48 (m, 2H), 2.17-2.04 (m, 1H), 1.83 (d, J = 13.8 Hz, 6H), 1.16 (t, J = 7.2 Hz, 3H), 0.90- 0.79 (m, 4H). LC-MS: 515.1 [M + H]⁺. |
| B26 | 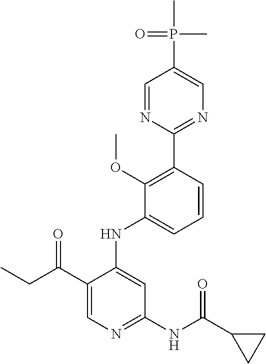 [SMILES: CCC(=O)c1cnc(NC(=O)C2CC2)cc1Nc1cccc(-c2ncc(P(C)(C)=O)cn2)c1OC] | AB, AC, AH, AK | ¹H NMR (300 MHz, DMSO-d₆): δ 11.51 (s, 1H), 10.51 (s, 1H), 9.30-9.16 (m, 2H), 8.09 (s, 1H), 7.67-7.60 (m, 2H), 7.40- 7.32 (m, 1H), 3.70 (s, 3H), 3.55-3.16 (m, 2H), 2.13-2.05 (m, 1H), 1.83 (d, J = 13.8 Hz, 6H), 1.16 (t, J = 6.6 Hz, 3H), 0.95- 0.74 (m, 4H). LC-MS: 495.2 [M + H]⁺. |
| B27 | 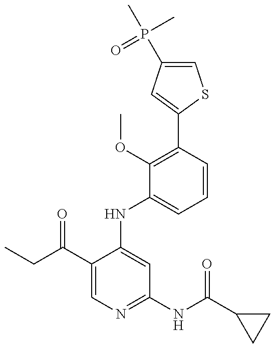 [SMILES: CCC(=O)c1cnc(NC(=O)C2CC2)cc1Nc1cccc(-c2cc(P(C)(C)=O)cs2)c1OC] | AA, AC, AH, AI | ¹H NMR (300 MHz, DMSO-d₆): δ 10.03 (s, 1H), 10.92 (s, 1H), 8.88 (s, 1H), 8.12 (d, J = 7.5 Hz, 1H), 7.99 (s, 1H), 7.91 (d, J = 3.6 Hz, 1H), 7.67 (d, J = 7.5 Hz, 1H), 7.43 (d, J = 7.9 Hz, 1H), 7.32-7.23 (m, 1H), 3.61 (s, 3H), 3.19-3.06 (m, 2H), 2.06-1.93 (m, 1H), 1.69 (d, J = 13.5 Hz, 6H), 1.12 (t, J = 6.9 Hz, 3H), 0.80-0.71 (m, 4H). LC-MS: 498.1 [M + H]⁺. |
| B28 | 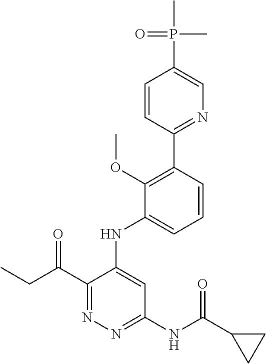 [SMILES: CCC(=O)c1nnc(NC(=O)C2CC2)cc1Nc1cccc(-c2ccc(P(C)(C)=O)cn2)c1OC] | AB, AG, AH, AK | ¹H NMR (300 MHz, DMSO-d₆): δ 11.51 (s, 1H), 10.55 (s, 1H), 9.03 (d, J = 5.7 Hz, 1H), 8.27-8.22 (m, 1H), 8.12 (s, 1H), 7.99 (d, J = 8.4 Hz, 1H), 7.62 (d, J = 7.5 Hz, 1H), 7.56 (d, J = 7.5 Hz, 1H), 7.43- 7.24 (m, 1H), 3.49 (s, 3H), 3.45-3.23 (m, 2H), 2.17-2.01 (m, 1H), 1.77 (d, J = 13.8 Hz, 6H), 1.16 (t, J = 7.2 Hz, 2H), 0.86- 0.81 (m, 4H). LC-MS: 494.1 [M + H]⁺. |
| B29 | 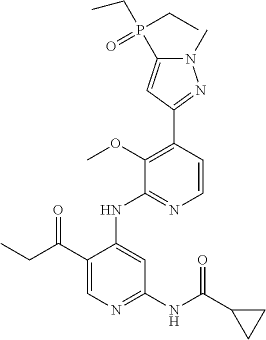 [SMILES: CCC(=O)c1cnc(NC(=O)C2CC2)cc1Nc1nccc(-c2cc(P(=O)(CC)CC)n(C)n2)c1OC] | AA, AE, AM | ¹H NMR (300 MHz, DMSO-d₆): δ 12.34 (s, 1H), 10.90 (s, 1H), 9.67 (s, 1H), 8.93 (s, 1H), 8.08 (d, J = 5.4 Hz, 1H), 7.43 (d, J = 5.4 Hz, 1H), 7.24 (s, 1H), 4.18 (s, 3H), 3.74 (s, 3H), 3.15 (dd, J = 13.8, 6.6 Hz, 2H), 2.15-1.95 (m, 5H), 1.17-0.96 (m, 9H), 0.87-0.75 (m, 4H). LC-MS: 525.2 [M + H]⁺. |
| B30 | 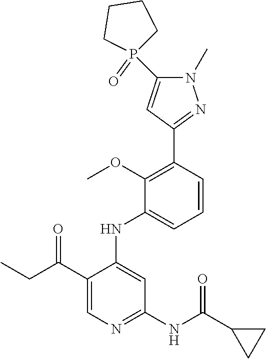 [SMILES: CCC(=O)c1cnc(NC(=O)C2CC2)cc1Nc1cccc(-c2cc(P3(=O)CCCC3)n(C)n2)c1OC] | AA, AC, AE, AF, AI | ¹H NMR (300 MHz, DMSO-d₆): δ 11.09 (s, 1H), 10.91 (s, 1H), 8.88 (s, 1H), 8.04 (s, 1H), 7.65 (d, J = 7.8 Hz, 1H), 7.44 (d, J = 7.8 Hz, 1H), 7.34-7.18 (m, 1H), 7.11 (s, 1H), 4.15 (s, 3H), 3.58 (s, 3H), 3.13 (q, J = 7.2 Hz, 2H), 2.37-2.18 (m, 2H), 2.13- 1.70 (m, 7H), 1.12 (t, J = 7.2 Hz, 3H), 0.86- 0.72 (m, 4H). LC-MS: 522.2 [M + H]⁺. |
| B31 | 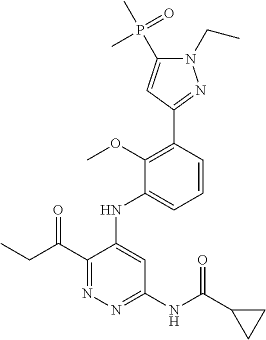 [SMILES: CCC(=O)c1nnc(NC(=O)C2CC2)cc1Nc1cccc(-c2cc(P(C)(C)=O)n(CC)n2)c1OC] | AB, AC, AE, AF, AK | ¹H NMR (300 MHz, DMSO-d₆): δ 11.48 (s, 1H), 10.56 (s, 1H), 8.09 (s, 1H), 7.76- 7.67 (m, 1H), 7.46-7.38 (m, 1H), 7.31- 7.21 (m, 1H), 7.05 (s, 1H), 4.60-4.48 (m, 2H), 3.61 (s, 3H), 3.50-3.47 (m, 2H), 2.11-2.03 (m, 1H), 1.82 (d, J = 14.1 Hz, 6H), 1.53-1.36 (m, 3H), 1.21-1.11 (m, 2H), 0.93-0.69 (m, 4H). LC-MS: 511.4 [M + H]⁺. |
| B32 | 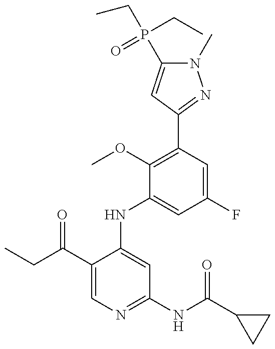 [SMILES: CCC(=O)c1cnc(NC(=O)C2CC2)cc1Nc1cc(F)cc(-c2cc(P(=O)(CC)CC)n(C)n2)c1OC] | AA, AC, AE, AF, AI | ¹H NMR (300 MHz, DMSO-d₆): δ 11.25 (s, 1H), 10.99 (s, 1H), 8.91 (s, 1H), 8.14 (s, 1H), 7.38 (dd, J = 9.9, 3.0 Hz, 1H), 7.32 (dd, J = 9.9, 3.3 Hz, 1H), 7.13 (s, 1H), 4.18 (s, 3H), 3.58 (s, 3H), 3.13 (q, J = 7.2 Hz, 2H), 2.20-1.89 (m, 5H), 1.25-0.91 (m, 9H), 0.87-0.75 (m, 4H). LC-MS: 542.2 [M + H]⁺. |
| B33 | 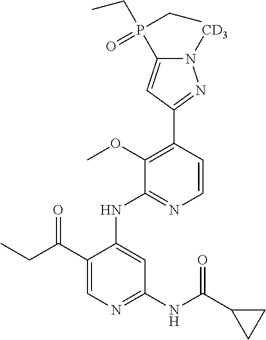 [SMILES: [2H]C([2H])([2H])n1nc(-c2ccnc(Nc3cc(NC(=O)C4CC4)ncc3C(=O)CC)c2OC)cc1P(=O)(CC)CC] | AA, AE, AM | ¹H NMR (300 MHz, DMSO-d₆): δ 12.34 (s, 1H), 10.90 (s, 1H), 9.67 (s, 1H), 8.93 (s, 1H), 8.08 (d, J = 5.4 Hz, 1H), 7.43 (d, J = 5.4 Hz, 1H), 7.23 (s, 1H), 3.73 (s, 3H), 3.14 (q, J = 7.4 Hz, 2H), 2.13-1.97 (m, 5H), 1.15-0.97 (m, 9H), 0.87-0.74 (m, 4H). LC-MS: 528.2 [M + H]⁺. |
| B34 | 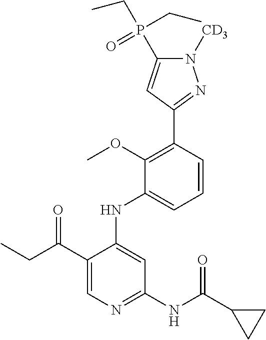 [SMILES: [2H]C([2H])([2H])n1nc(-c2cccc(Nc3cc(NC(=O)C4CC4)ncc3C(=O)CC)c2OC)cc1P(=O)(CC)CC] | AA, AC, AE, AF, AI | ¹H NMR (300 MHz, DMSO-d₆): δ 11.10 (s, 1H), 10.91 (s, 1H), 8.87 (s, 1H), 8.04 (s, 1H), 7.66 (d, J = 7.8 Hz, 1H), 7.44 (d, J = 7.9 Hz, 1H), 7.23 (t, J = 8.7 Hz, 1H), 7.07 (s, 1H), 3.56 (s, 4H), 3.17-3.06 (m, 2H), 2.11-1.95 (m, 4H), 1.34-0.89 (m, 9H), 0.87-0.67 (m, 4H). LC-MS: 527.2 [M + H]⁺. |
| B35 | 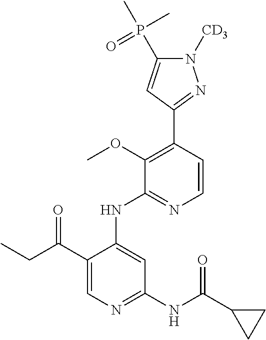 [SMILES: [2H]C([2H])([2H])n1nc(-c2ccnc(Nc3cc(NC(=O)C4CC4)ncc3C(=O)CC)c2OC)cc1P(C)(C)=O] | AA, AE, AM | ¹H NMR (300 MHz, DMSO-d₆): δ 12.34 (s, 1H), 10.91 (s, 1H), 9.68 (s, 1H), 8.95 (s, 1H), 8.10 (d, J = 5.1 Hz, 1H), 7.45 (d, J = 4.8 Hz, 1H), 7.26 (s, 1H), 3.78 (s, 3H), 3.16 (q, J = 6.9 Hz, 2H), 2.11-2.00 (m, 4H), 1.85 (d, J = 13.8 Hz, 6H), 1.13 (t, J = 6.6 Hz, 3H), 0.93-0.74 (m, 4H). LC-MS: 500.2 [M + H]⁺. |
| B36 | 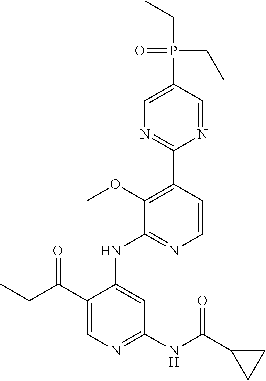 [SMILES: CCC(=O)c1cnc(NC(=O)C2CC2)cc1Nc1nccc(-c2ncc(P(=O)(CC)CC)cn2)c1OC] | AA, AG, AM | ¹H NMR (300 MHz, DMSO-d₆): δ 12.38 (s, 1H), 10.94 (s, 1H), 9.72 (s, 1H), 9.26 (d, J = 5.1 Hz, 2H), 8.97 (s, 1H), 8.22 (d, J = 4.8 Hz, 1H), 7.43 (d, J = 5.0 Hz, 1H), 3.83 (s, 3H), 3.21-3.09 (m, 2H), 2.21- 2.02 (m, 5H), 1.16-0.96 (m, 9H), 0.89- 0.80 (m, 4H). LC-MS: 523.2 [M + H]⁺. |
| B37 | 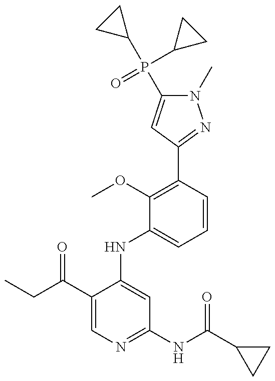 [SMILES: CCC(=O)c1cnc(NC(=O)C2CC2)cc1Nc1cccc(-c2cc(P(=O)(C3CC3)C3CC3)n(C)n2)c1OC] | AA, AC, AF, AJ, AI | ¹H NMR (300 MHz, CDCl₃): δ 11.31 (s, 1H), 8.73 (s, 1H), 8.36 (s, 1H), 8.14 (s, 1H), 7.69 (d, J = 7.5 Hz, 1H), 7.52 (d, J = 7.8 Hz, 1H), 7.30-7.24 (m, 1H), 7.22 (s, 1H), 4.25 (s, 3H), 3.65 (s, 3H), 3.05 (q, J = 7.2 Hz, 2H), 1.62-1.46 (m, 1H), 1.27 (t, J = 7.2 Hz, 3H), 1.14-0.82 (m, 14H). LC-MS: 547.8 [M + H]⁺. |
| B38 | 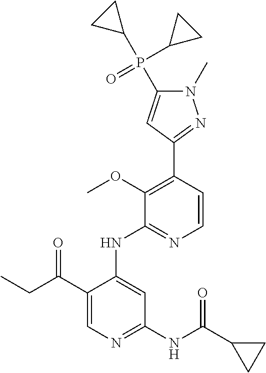 [SMILES: CCC(=O)c1cnc(NC(=O)C2CC2)cc1Nc1nccc(-c2cc(P(=O)(C3CC3)C3CC3)n(C)n2)c1OC] | AA, AE, AM | ¹H NMR (300 MHz, DMSO-d₆): δ 12.35 (s, 1H), 10.92 (s, 1H), 9.69 (s, 1H), 8.95 (s, 1H), 8.10 (d, J = 5.1 Hz, 1H), 7.47 (d, J = 5.1 Hz, 1H), 7.40 (s, 1H), 4.15 (s, 3H), 3.78 (s, 3H), 3.23-3.07 (m, 2H), 2.14- 1.97 (m, 1H), 1.51-1.32 (m, 2H), 1.12 (t, J = 6.9 Hz, 3H), 0.98-0.90 (m, 4H), 0.87- 0.74 (m, 8H). LC-MS: 549.2 [M + H]⁺. |
| B39 | 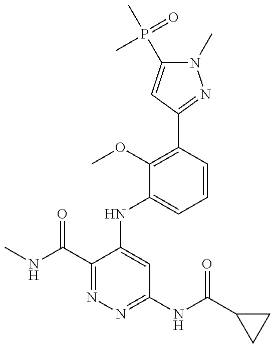 [SMILES: CNC(=O)c1nnc(NC(=O)C2CC2)cc1Nc1cccc(-c2cc(P(C)(C)=O)n(C)n2)c1OC] | AB, AC, AE, AF, AL | ¹H NMR (300 MHz, DMSO-d₆): δ 11.32 (s, 1H), 11.01 (s, 1H), 9.17 (s, 1H), 8.15 (s, 1H), 7.66 (d, J = 7.8 Hz, 1H), 7.42 (d, J = 7.5 Hz, 1H), 7.29-7.18 (m, 1H), 7.07 (s, 1H), 4.16 (s, 3H), 3.61 (s, 3H), 2.86 (d, J = 4.2 Hz, 3H), 2.13-2.01 (m, 1H), 1.82 (d, J = 13.8 Hz, 6H), 0.87-0.75 (m, 4H). LC-MS: 497.8 [M + H]⁺. |
| B40 | 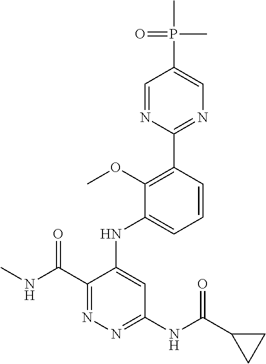 [SMILES: CNC(=O)c1nnc(NC(=O)C2CC2)cc1Nc1cccc(-c2ncc(P(C)(C)=O)cn2)c1OC] | AB, AC, AH, AL | ¹H NMR (300 MHz, DMSO-d₆): δ 11.34 (s, 1H), 10.95 (s, 1H), 9.25 (s, 1H), 9.23 (s, 1H), 9.21-9.14 (m, 1H), 8.15 (s, 1H), 7.65- 7.57 (m, 2H), 7.40-7.29 (m, 1H), 3.72 (s, 3H), 2.86 (d, J = 4.2 Hz, 3H), 2.14-2.02 (m, 1H), 1.83 (d, J = 13.8 Hz, 6H), 0.87- 0.77 (m, 4H). LC-MS: 496.2 [M + H]⁺. |
| B41 | 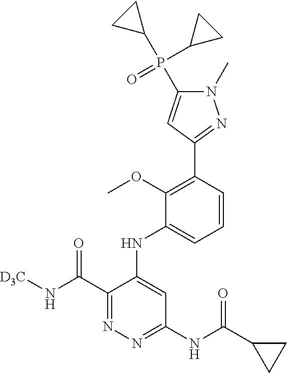 | AB, AC, AE, AF, AL | ¹H NMR (300 MHz, DMSO-d₆): δ 11.33 (s, 1H), 11.03 (s, 1H), 9.15 (s, 1H), 8.17 (s, 1H), 7.69 (d, J = 8.1 Hz, 1H), 7.43 (d, J = 7.8 Hz, 1H), 7.33-7.15 (m, 2H), 4.13 (s, 3H), 3.62 (s, 3H), 2.16-1.95 (m, 1H), 1.45-1.23 (m, 2H), 1.00-0.86 (m, 4H), 0.86-0.73 (m, 8H). LC-MS: 511.4 [M + H]⁺. |
| B42 | 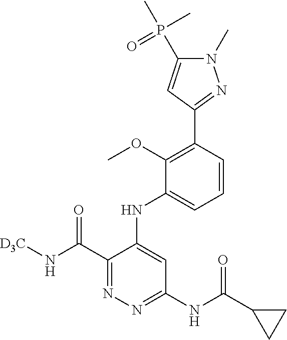 [SMILES: [2H]C([2H])([2H])NC(=O)c1nnc(NC(=O)C2CC2)cc1Nc1cccc(-c2cc(P(C)(C)=O)n(C)n2)c1OC] | AB, AC, AE, AF, AL | ¹H NMR (300 MHz, DMSO-d₆): δ 11.33 (s, 1H), 11.03 (s, 1H), 9.15 (s, 1H), 8.16 (s, 1H), 7.67 (d, J = 7.8 Hz, 1H), 7.42 (d, J = 7.8 Hz, 1H), 7.31-7.20 (m, 1H), 7.08 (s, 1H), 4.16 (s, 3H), 3.61 (s, 3H), 2.11-2.05 (m, 1H), 1.82 (d, J = 13.8 Hz, 6H), 0.85- 0.78 (m, 4H). LC-MS: 500.8 [M + H]⁺. |
| B43 | 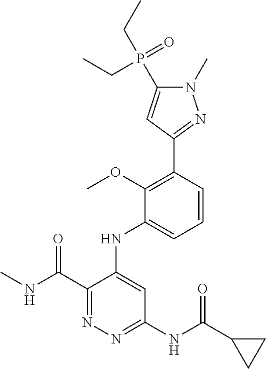 [SMILES: CCP(=O)(CC)c1cc(-c2cccc(Nc3cc(NC(=O)C4CC4)nnc3C(=O)NC)c2OC)nn1C] | AB, AC, AE, AF, AL | ¹H NMR (300 MHz, DMSO-d₆): δ 11.32 (s, 1H), 11.03 (s, 1H), 9.16 (s, 1H), 8.17 (s, 1H), 7.66 (d, J = 7.8 Hz, 1H), 7.42 (d, J = 7.8 Hz, 1H), 7.32-7.22 (m, 1H), 7.06 (s, 1H), 4.17 (s, 3H), 3.58 (s, 3H), 2.86 (d, J = 3.6 Hz, 3H), 2.15-1.93 (m, 5H), 1.16- 0.97 (m, 6H), 0.90-0.70 (m, 4H). LC-MS: 525.8 [M + H]⁺. |
| B44 | 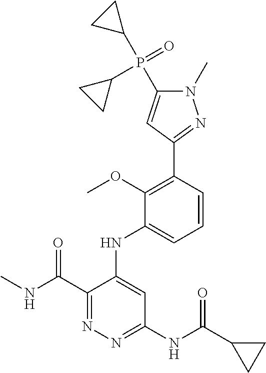 [SMILES: CNC(=O)c1nnc(NC(=O)C2CC2)cc1Nc1cccc(-c2cc(P(=O)(C3CC3)C3CC3)n(C)n2)c1OC] | AB, AC, AE, AF, AL | ¹H NMR (300 MHz, DMSO-d₆): δ 11.33 (s, 1H), 11.03 (s, 1H), 9.17 (s, 1H), 8.18 (s, 1H), 7.69 (d, J = 7.2 Hz, 1H), 7.43 (d, J = 7.8 Hz, 1H), 7.29-7.20 (m, 2H), 4.13 (s, 3H), 3.62 (s, 3H), 2.98-2.76 (m, 3H), 2.13-2.03 (m, 1H), 1.41-1.28 (m, 2H), 0.99-0.87 (m, 4H), 0.87-0.73 (m, 8H). LC-MS: 549.8 [M + H]⁺. |
| B45 | 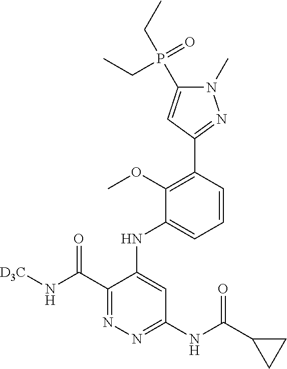 [SMILES: [2H]C([2H])([2H])NC(=O)c1nnc(NC(=O)C2CC2)cc1Nc1cccc(-c2cc(P(=O)(CC)CC)n(C)n2)c1OC] | AB, AC, AE, AF, AL | ¹H NMR (300 MHz, DMSO-d₆): δ 11.30 (s, 1H), 11.03 (s, 1H), 9.12 (s, 1H), 8.16 (s, 1H), 7.67 (d, J = 7.8 Hz, 1H), 7.42 (d, J = 7.2 Hz, 1H), 7.31-7.18 (m, 1H), 7.06 (s, 1H), 4.17 (s, 3H), 3.58 (s, 3H), 2.20-1.95 (m, 5H), 1.22-0.97 (m, 6H), 0.93-0.68 (m, 4H). LC-MS: 528.8 [M + H]⁺. |
| B46 | 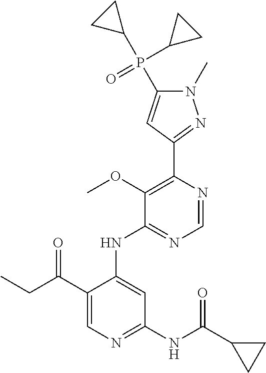 [SMILES: CCC(=O)c1cnc(NC(=O)C2CC2)cc1Nc1ncnc(-c2cc(P(=O)(C3CC3)C3CC3)n(C)n2)c1OC] | AA, AJ, AN | ¹H NMR (300 MHz, DMSO-d₆): δ 12.59 (s, 1H), 11.06 (s, 1H), 9.78 (s, 1H), 9.03 (s, 1H), 8.63 (s, 1H), 7.44 (s, 1H), 4.20 (s, 3H), 3.93 (s, 3H), 3.19 (q, J = 6.9 Hz, 2H), 2.14-2.03 (m, 1H), 1.45-1.32 (m, 2H), 1.14 (t, J = 7.2 Hz, 3H), 0.97-0.73 (m, 12H). LC-MS: 549.8 [M + H]⁺. |

### Example 15, Competitive Binding Assay

### Experimental Procedure:

For most assays, kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111X stocks in 100% DMSO. Dissociation constants (Kds) were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM nonbiotinylated affinity ligand) and incubated at room temperature with shaking for 30 minutes. The kinase concentration in the eluates was measured by qPCR.

An 11-point 3-fold serial dilution of each test compound was prepared in 100% DMSO at 100× final test concentration and subsequently diluted to 1× in the assay (final DMSO concentration=1%). Most Kds were determined using a compound top concentration=30,000 nM. If the initial Kd determined was <0.5 nM (the lowest concentration tested), the measurement was repeated with a serial dilution starting at a lower top concentration. A Kd value reported as 40,000 nM indicates that the Kd was determined to be >30,000 nM.

Binding constants (Kds) were calculated with a standard dose-response curve using the Hill equation:

$$Response = Background + \frac{Signal - Background}{1 + ( {Kd}^{Hill Slope} / {Dose}^{Hill Slope} )}$$

The Hill Slope was set to −1.

Curves were fitted using a non-linear least square fit with the Levenberg-Marquardt algorithm.

TABLE 2
Competitive binding affinity of compounds to TYK2 JH2, JAK1 JH1 and JAK2 JH1

| | TYK2 JH2 | JAK1 JH1 | JAK2 JH1 | | TYK2 JH2 | JAK1 JH1 | JAK2 JH1 |
|---|---|---|---|---|---|---|---|
| NO. | Kd (nM) | Kd (nM) | Kd (nM) | NO. | Kd (nM) | Kd (nM) | Kd (nM) |
| B2 | 0.043 | >30000 | \ | B3 | 0.40 | >30000 | \ |
| B4 | 0.032 | >30000 | \ | B5 | 0.095 | >30000 | \ |
| B11 | 0.016 | >30000 | \ | B19 | 0.075 | \ | \ |
| B22 | 0.063 | \ | \ | B24 | 0.03 | \ | \ |
| B29 | 0.01 | \ | \ | B32 | 0.025 | \ | \ |
| B33 | 0.056 | \ | \ | B34 | 0.044 | \ | \ |
| B35 | 0.025 | \ | \ | B37 | 0.044 | >30000 | >30000 |
| B38 | 0.0079 | >30000 | 11000 | B39 | 0.0064 | \ | \ |
| B41 | 0.014 | >30000 | >30000 | | | | |

Conclusion: According to Table 2 and FIG. 2 (Compound B2), tested compounds B2-B5, B11, B19, B22, B24, B29, B32-B35, B37-B39 and B41 display strong affinity to the TYK2 pseudokinase domain and, at the same time, display almost no affinity to the JAK1 and JAK2 kinase domains.

### Example 16. Luciferase Assay

### Experimental Procedure:

1. L929 ISRE cells (5000 cells/well) were seeded on 96-well plates and incubated overnight at room temperature;
2. The cells were pretreated by adding different concentrations (10 nM, 50 nM, 100 nM, 200 nM or 1000 nM) of the test compounds for 2 hours;
3. Added IFN-α (100 ng/mL) to stimulate cells for 6 hours;
4. Removed the upper medium, add PBS (100 μL) and washed, then removed PBS;
5. Added PLB lysis solution (50 μL) to each well, shook the well for 15 minutes;
6. Transferred PLB lysis solution (30 μL) to a new 96-well whiteboard, added LAR II reagent (LAR II), then quickly read the 450 nm OD value with a plate washer;
7. Added Stop & Glo reagent (30 μL), then read the 450 nm OD value using the plate washer.
8. The final results in Table 3 were the OD value in step 6/OD value in step 7.

TABLE 3
Inhibitory activity of compounds on TYK2

| | Inhibitory | Inhibitory | Inhibitory | Inhibitory | Inhibitory |
|---|---|---|---|---|---|
| | activity % | activity % | activity % | activity % | activity % |
| NO. | (10 nM) | (50 nM) | (100 nM) | (200 nM) | (1000 nM) |
| B1 | 0 | \ | 0 | \ | 0 |
| B2 | 45 | \ | 58 | \ | 94 |
| B3 | 21 | \ | 46 | \ | 88 |
| B4 | \ | \ | \ | \ | 58 |
| B5 | \ | \ | \ | \ | 80 |
| B6 | \ | \ | \ | \ | 36 |
| B7 | \ | \ | \ | \ | 46 |
| B8 | \ | \ | \ | \ | 33 |
| B9 | \ | \ | \ | \ | 40 |
| B10 | \ | \ | \ | \ | 50 |
| B11 | \ | \ | \ | \ | 62 |
| B12 | \ | \ | \ | \ | 0 |
| B13 | 0 | \ | 17 | \ | 76 |
| B14 | \ | \ | \ | \ | 27 |
| B15 | \ | \ | \ | \ | 45 |
| B16 | 35 | \ | 38 | \ | 55 |
| B17 | 25 | \ | 45 | \ | 67 |
| B18 | \ | \ | \ | \ | 42 |
| B19 | 41 | \ | 74 | \ | 88 |
| B20 | 7 | \ | 8 | \ | 20 |
| B21 | \ | \ | 6 | \ | 49 |
| B22 | \ | \ | 43 | \ | 83 |
| B23 | \ | \ | 8 | \ | 46 |
| B24 | \ | \ | 31 | \ | 93 |
| B25 | \ | \ | \ | \ | 13 |
| B26 | \ | \ | \ | \ | 2 |
| B27 | \ | \ | \ | \ | 38 |
| B28 | \ | \ | \ | \ | 35 |
| B29 | \ | \ | \ | \ | 93 |
| B30 | \ | \ | \ | \ | 75 |
| B31 | \ | \ | \ | \ | 26 |
| B32 | \ | \ | \ | \ | 88 |
| B33 | \ | \ | \ | \ | 86 |
| B34 | \ | \ | \ | \ | 87 |
| B35 | \ | \ | \ | \ | 60 |
| B36 | \ | \ | \ | \ | 75 |
| B37 | \ | 89 | \ | 95 | 97 |
| B38 | \ | \ | \ | \ | 95 |
| B39 | \ | \ | 34 | \ | 80 |
| B40 | \ | \ | 33 | \ | 58 |
| B41 | \ | 86 | \ | 92 | 97 |
| B42 | \ | \ | \ | \ | 65 |
| B43 | \ | \ | \ | \ | 84 |
| B44 | \ | \ | \ | \ | 81 |
| B45 | \ | 44 | \ | 83 | 95 |
| B46 | \ | 70 | \ | 88 | 96 |
| BMS986165 | \ | 74 | \ | 89 | 96 |

The symbol “\” indicates not detected; BMS986165 is the reference compound for comparison.

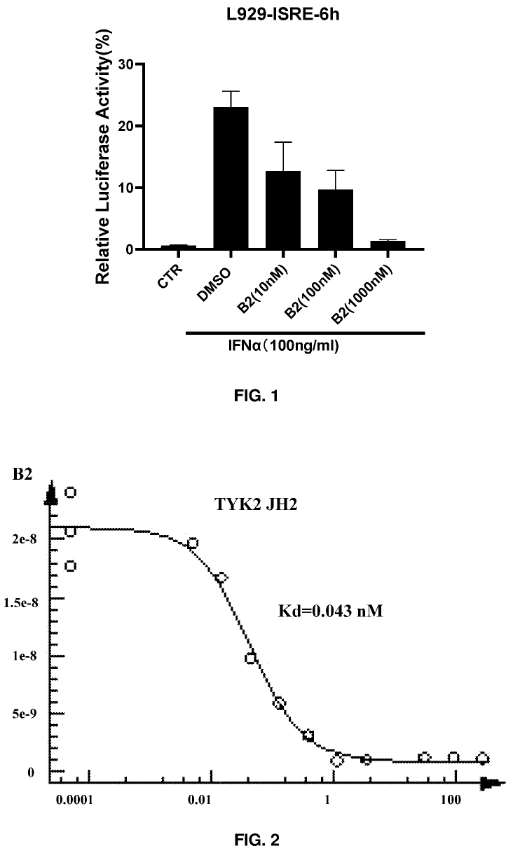

According to Table 3 and FIG. 1 compounds B2-B5, B10, B11, B13, B16, B17, B19, B22, B24, B29, B30, and B32-B46 displayed good inhibitory activities against TYK2.

### Example 17, Enzyme-Linked Immunosorbent Assays (ELISA)

### Experimental Procedure:

1. Soak plate: Add 300 μL 1×washing solution and let stand for 30 seconds. After discarding the washing solution, dry the microporous plate on absorbent paper.
2. Add standard: Add 100 μL of 2-fold standard to standard well. Add 100 μL standard dilution (serum/plasma sample) or culture medium (cell culture supernatant sample) to blank well.
3. Add sample: Serum/plasma: Add 50 μL 1×assay buffer and 50 μL sample to sample well. Cell culture supernatant: Add 100 μL cell culture supernatant to sample well.
4. Add detection antibody: Add 50 μL diluted detection antibody (1:100 diluted) to each well. Make sure steps 4, 5 and 6 are added continuously without interruption. The sampling process was completed within 15 minutes.
5. Incubation: Use sealing plate film to seal the plate. shake at 300 rpm and incubate for 2 hours at room temperature.
6. Washing: Discard the liquid, add 300 μL of washing solution and wash the plate 6 times. pat dry on blotting paper after each wash. To obtain the desired experimental performance, the residual liquid must be completely removed.
7. Enzyme addition: Add 100 μL diluted horseradish peroxidase labeled streptavidin to each well (1:100 diluted).
8. Incubation: Seal the plate with a new sealing film. shake at 300 rpm and incubate at room temperature for 45 minutes.
9. Washing. Repeat step 8.
10. Add substrate for color development: Add 100 μL of color development substrate TMB to each well, keep away from light, and incubate at room temperature for 5-30 minutes.
11. Add Stop Solution: Add 100 μL stop solution to each well. The color changed from blue to yellow. If the color appears green or the color changes unevenly, gently tap the frame of the plate to mix well.
12. Assay readout: Within 30 minutes, perform dual wavelength detection using a microplate reader to measure the OD at the absorption maximum at 450 nm and reference wavelengths at 570 nm or 630 nm. The calibrated OD value is the measured value at 450 nm minus the measured value at 570 nm or 630 nm.

TABLE 4
ELISA results of test compounds.

| NO. | IC₅₀ (nM) | NO. | IC₅₀ (nM) |
|---|---|---|---|
| B37 | 28.2 | B38 | 221.9 |
| B41 | 39.5 | BMS986165 | 56.8 |

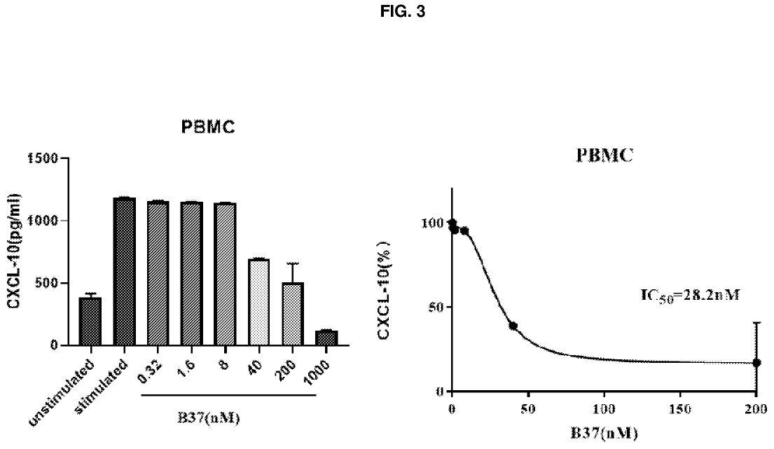

The results were shown in Table 4 and FIG. 3. Compounds B37, B38, B41 and BMS986165 displayed inhibition against the production of CXCL-10 stimulated by IFNα in PBMC. Compounds B37 and B41 were both more active than BMS986165.

### Example 18, Selectivity Evaluation

Goal 1: To test the inhibitory activity of JAK2/JAK2 pathway

### Experimental Procedure:

MEG-1 cells were digested by trypsin, re-suspended in 1640 medium and counted. The compounds to be measured were added to 6 well plates (1 million/per well) for 2 hours, and then TPO (final concentration: 100 ng/mL) was added to stimulate cells for 30 minutes. The cells were collected and lysed with protein lysate. The protein was quantitatively tested by BCA kit. Finally, p-STAT3/STAT3 and internal reference protein β-actin were detected by western blot.

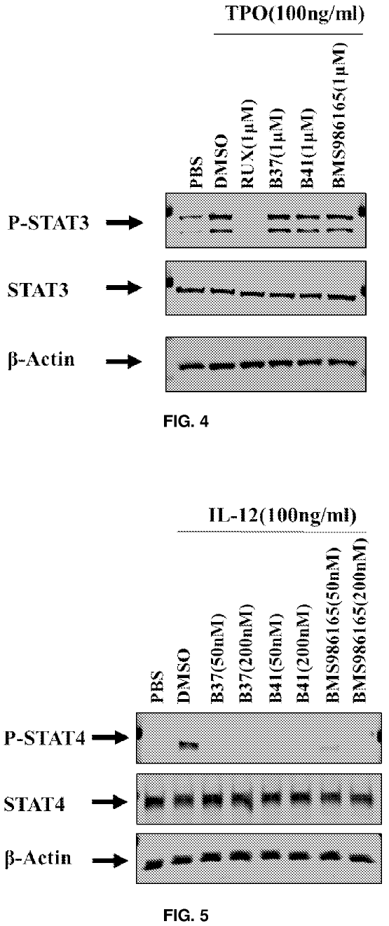

As shown in FIG. 4, JAK1/JAK2 inhibitor Ruxolitinib (RUX in figure) could inhibit JAK2/JAK2 pathway. Compounds B37 and B41 which had no effect on TPO-induced downstream STAT3 phosphorylation were similar to BMS986165, thereby indicating that there was no inhibitory activity on JAK2/JAK2 pathway. Therefore, compounds B37 and B41 had no inhibitory activity on JAK2.

Goal 2: To test the inhibitory activity of TYK2/JAK2 pathway

### Experimental Procedure:

The spleen of mice was isolated and ground into single cells with bent tweezers. The single cells of the spleen were centrifuged for 5 minutes (400 g), and erythrocytes were lysed with ACK solution and neutralized in 1640 medium, centrifuged for 5 minutes (400 g), and then re-suspended in 1640 medium. The spleen single cell suspension was counted and incubated to a 6-well plate (5 million/well), the compound to be tested was added for 2 hours, and then IL-12 (50 ng/mL) was added to stimulate cells for 1 h. The cells were collected and lysed with protein lysate. The protein was quantitatively tested by BCA kit. Finally, p-STAT4/STAT4 and internal reference protein β-actin were detected by western blot.

As shown in FIG. 5, compounds B37 and B41 could effectively inhibit the downstream STAT4 phosphorylation induced by IL-12, indicating that they could inhibit the TYK2/JAK2 pathway, and their inhibition activities were better than BMS986165. Combined with the results in FIG. 4, which indicates that compounds B37 and B41 did not inhibit JAK2, their inhibitory activities on TYK2/JAK2 pathway may come from the inhibition of TYK2. Overall, the activity of B37 and B41 on TYK2 was superior to BMS986165.

Goal 3: To test the inhibitory activity of TYK2/JAK1 pathway

### Experimental Procedure:

Commercial human PBMC (peripheral blood mononuclear cells) were centrifuged (400 g) for 10 min, counted and planted on 6-well plates (5 million/well). Then, the compounds to be tested were added for 2 hours and IFN-α (100 ng/mL) was added to stimulate cells for 15 minutes. Protein lysate was used to lysate cells, and BCA kit was used for protein quantification. Finally, p-STAT1/STAT1, P-TYK2/TYK2 and internal reference protein B-Actin were detected by Western blot.

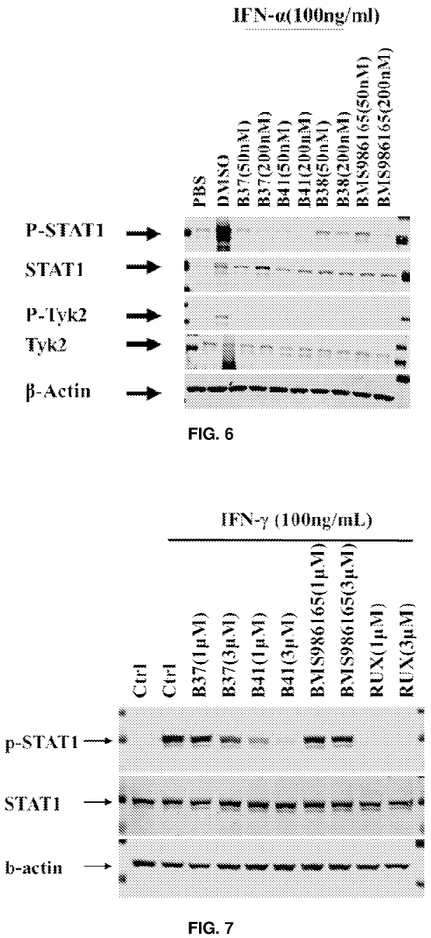

As shown in FIG. 6, compounds B37, B38 and B41 could effectively inhibit the downstream STAT1 phosphorylation induced by IFN-α, indicating that they could inhibit the TYK2/JAK1 pathway. Among them, compounds B37 and B41 displayed better inhibitory activity than BMS986165.

Goal 4: To test the inhibitory activity of JAK1/JAK2 pathway

### Experimental Procedure:

HT-29 cells were digested by trypsin and suspended in DMEM medium and counted. The tested compounds were added into 6 well plates (400 000/per well) for 2 hours, then IFN-γ (100 ng/mL) was added to stimulate cells for 30 minutes. The cells were collected and lysed with protein lysate. The protein was quantitatively tested by BCA kit. Finally, p-STAT1/STAT1 and internal reference protein β-actin were detected by western blot.

As shown in FIG. 7, compound B37 was similar to BMS986165 and had no effect on downstream STAT1 phosphorylation induced by IFN-γ, indicating that it had no inhibitory effect on JAK1/JAK2 pathway, while compound B41 inhibited JAK1/JAK2 pathway in a concentration-dependent manner. Combined with the results in FIG. 4, compound B41 had no inhibitory activity on JAK2, indicating that the inhibitory effect of B41 on JAK1/JAK2 pathway came from the inhibition of JAK1. As a consequence, B41 displayed higher biological activities than BMS986165. Therefore, compound B41 or other compounds disclosed herein could be used to treat autoimmune diseases.

Goal 5: To test the inhibitory activity of JAK1/JAK3 pathway

### Experimental Procedure:

Commercial human PBMC (peripheral blood mononuclear cells) were centrifuged (400 g) for 10 min, counted and seeded into 6-well plates (5 million/well). The compounds to be tested were added for 2 hours and then IL-2 (100 ng/mL) was added to stimulate cells for 15 minutes. Finally, p-STAT5/STAT5 and internal reference protein β-actin were detected by Western blot.

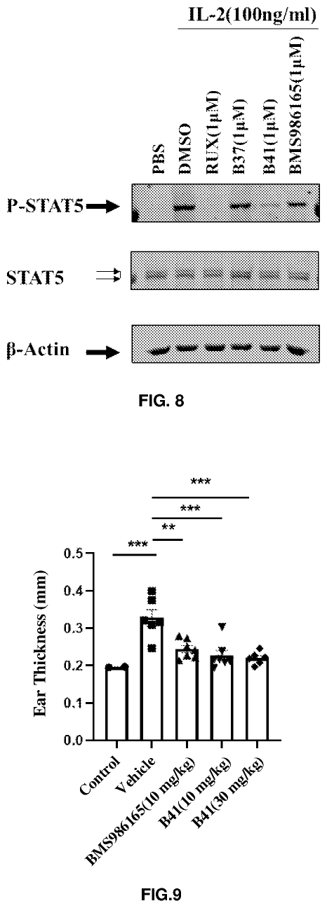

As shown in FIG. 8, compound B37 was similar to BMS986165 and had no effect on IL-2-induced downstream STAT5 phosphorylation, indicating that it had no inhibitory activity on JAK1/JAK3 pathway, while compound B41 could inhibit STAT5 phosphorylation. The inhibitory activity of B41 was slightly lower than JAK1/JAK2 inhibitor Ruxolitinib (denoted as RUX in FIG. 8), indicating that B41 displayed an inhibitory activity on JAK1/JAK3 pathway. The experimental results proved that compound B41 had inhibitory activity on JAK1 again.

### Example 19, Metabolic Stability Test

### Experimental Procedure:

1. Buffer A: 1.0 L of 0.1 M monobasic Potassium Phosphate buffer containing 1.0 mM EDTA
   Buffer B: 1.0 L of 0.1 M Dibasic Potassium Phosphate buffer containing 1.0 mM EDTA
   Buffer C: 0.1 M Potassium Phosphate buffer, 1.0 mM EDTA, at about pH 7.4 by titrating 700 mL of Buffer B with Buffer A while monitoring with the pH meter.
2. Reference compounds (Ketanserin) and test compounds spiking solution:
   500 UM spiking solution: add 10 μL of 10 mM DMSO stock solution into 190 pL ACN.
   1.5 UM spiking solution in microsomes (0.75 mg/mL): add 1.5 μL of 500 UM spiking solution and 18.75 μL of 20 mg/mL liver microsomes into 479.75 μL of Buffer C on ice.
3. Prepare NADPH stock solution (6 mM) by dissolving NADPH into buffer C.
4. Dispense 30 μL of 1.5 UM spiking solution containing 0.75 mg/mL microsomes solution to the assay plates designated for different time points (0-, 5-, 15-, 30-, 45-min) on ice.
5. For 0-min, add 135 μL of ACN containing IS to the wells of 0-min plate and then add 15 μL of NADPH stock solution (6 mM).
6. Pre-incubate all other plates at 37° C. for 5 minutes.
7. Add 15 μL of NADPH stock solution (6 mM) to the plates to start the reaction and timing.
8. At 5-min, 15-min, 30-min, and 45-min, add 135 μL of ACN containing IS to the wells of corresponding plates, respectively, to stop the reaction.
9. After quenching, shake the plates at the vibrator (IKA, MTS 2/4) for 10 min (600 rpm/min) and then centrifuge at 5594 g for 15 min (Thermo Multifuge×3R).
10. Transfer 50 μL of the supernatant from each well into a 96-well sample plate containing 50 μL of ultra pure water (Millipore, ZMQS50F01) for LC/MS analysis.

TABLE 5
Metabolic stability of test compounds

| | T_{1/2} (min) | | CL (mL/min/kg) | |
|---|---|---|---|---|
| NO. | HLM | RLM | HLM | RLM |
| B2 | >145 | 101 | <8.6 | 24.6 |

Compound B2 had good stability in human and rat liver microsomes shown according to Table 5.

### Example 20, Kinetic Solubility Test

### Experimental Procedure:

1. 10 μL of test and control compounds (10 mM) in DMSO was added into lower chambers of Whatman mini-uniprep vials, respectively.
2. Added 490 μL of 50 mM PB (pH 7.4) into lower chambers of the Whatman mini-uniprep vials, respectively.
3. Vortexed the solubility samples for at least 2 minutes.
4. Shook the mini-uniprep vials on a Barnstead shaker for 2 hours at room temperature at the speed of 800 rpm.
5. Centrifuged 20 minutes (e.g. 4000 rpm)
6. Compressed mini-unipreps to prepare the filtrates for injection into UPLC system to calculate the concentration with standard curve.

TABLE 6
Kinetic solubility test results

| NO. | Kinetic solubility (pH 7.4) |
|---|---|
| B2 | 52.4 μM |
| B22 | 91.9 μM |
| B41 | 11.3 μM |

As shown in Table 6, compounds B2 and B22 had good kinetic solubility. Compound B41 has low to moderate kinetic solubility.

### Example 21, Plasma Protein Binding Test

### Experimental Procedure:

1. Test compound and control compound were dissolved in dimethyl sulfoxide (DMSO) to achieve 10 mM stock solutions. DMSO working solutions were prepared at 400 μM. To prepare the loading matrix, compound working solutions (5 μL) were added in a 1:200 ratio to blank matrix (995 μL), and mixed thoroughly.
2. To prepare the time zero (TO) samples to be used for recovery determination, 50 μL aliquots of loading matrix were transferred in triplicate to the sample collection plate. The samples were immediately matched with opposite blank buffer to obtain a final volume of 100 μL of 1:1 matrix/dialysis buffer (v/v) in each well. 500 μL of stop solution were added to these TO samples. They were then stored at 2-8° C. pending further processes along with other post-dialysis samples.
3. To load the dialysis device, an aliquot of 150 μL of the loading matrix was transferred to the donor side of each dialysis well in triplicate, and 150 μL of the dialysis buffer was loaded to the receiver side of the well. The dialysis plate was placed in a humidified incubator at 37° C. with 5% CO₂ on a shaking platform that rotated slowly (about 100 rpm) for 4 hours
4. At the end of the dialysis, aliquots of 50 μL of samples were taken from both the buffer side and the matrix side of the dialysis device. These samples were transferred into new 96-well plates. Each sample was mixed with an equal volume of opposite blank matrix (buffer or matrix) to reach a final volume of 100 μL of 1:1 matrix/dialysis buffer (v/v) in each well. All samples were further processed by adding 500 μL of stop solution containing internal standards. The mixture was vortexed and centrifuged at 4000 rpm for about 20 minutes. An aliquot of 100 μL of supernatant of all the samples was then removed for LC-MS/MS analysis
5. The single blank samples were prepared by transferring 50 pL of blank matrix to a 96 well plate and adding 50 μL of blank PBS buffer to each well. The blank plasma must match the species of plasma used in the plasma side of the well. Then the matrix-matched samples were further processed by adding 500 μL of stop solution containing internal standards, following the same sample processing method as the dialysis samples.

### Data Calculation:

The % Unbound and % Bound were calculated using the following equations:

% Unbound=100×[F]/[T]; % Bound=100−% Unbound

Where [F] is the analyte concentration or peak area ratio of analyte/internal standard on the buffer (receiver) side of the membrane, [T] is the analyte concentration or peak area ratio of analyte/internal standard on the matrix (donor) side of the membrane, and [T0] is the analyte concentration or the peak area ratio of analyte/internal standard in the loading matrix sample at time zero.

TABLE 7
Plasma protein binding test results

| | Plasma protein binding(%) | | | | |
|---|---|---|---|---|---|
| NO. | Human | Rat | Mouse | Dog | Monkey |
| B2 | 95.2 | 94.5 | 93.3 | 88.1 | 95.8 |
| B22 | 94.9 | \ | 97.0 | 84.4 | \ |
| B41 | 91.6 | 95.5 | 98.2 | 98.1 | 92.4 |

According to Table 7, compounds B2, B22, B41 had moderate to high plasma protein binding.

### Example 22, hERG Channel Inhibition Activity Test

### Experimental Procedure:

Chinese hamster ovary (CHO) cell line, CHO-hERG cells were used in this study. CHO-hERG cells were derived from Sophion Biosciences Inc. (Ballerup, Denmark) in Suzhou, China. The cell reserve fluid is frozen in liquid nitrogen tanks. Each batch of stored cell fluid is tested for mycoplasma contamination. The cells were no longer used after 30 generations.

The whole cell patch clamp technique was used to record hERG current at room temperature. The patch-clamp amplifier outputs signals through digital to analog conversion and 2.9 KHz low-pass filtering. Data records were collected by Patchmaster Pro software.

Cells were planted in a cell recording tank and placed on an inverted microscope platform. One cell in the recording tank was randomly selected for experiment. The perfusion system was fixed on an inverted microscope platform and cells were continuously irrigated with ECS.

Manual patch clamp recording microelectrode was prepared with capillary glass tube, which was filled with intracellular fluid. On the day of the patch clamp test, the electrodes were prepared using a borosilicate glass tube (GC150TF-10, Harvard Apparatus Co. UK). The resistance is between 2-5 MΩ after the electrode is filled with ICS.

The clamping voltage was −80 mV, and the first step was depolarized to +60 mV for 850 ms to open the hERG channel. The voltage is then set to −50 mV and maintained for 1275 ms, resulting in a rebound current or tail current, the peak of which is measured and used for analysis. Finally, the voltage is restored to the clamping voltage (−80 mV). During the test, the command voltage program was repeated every 15 s.

At the beginning of recording the perfusion of the solvent control working solution, the test substance/positive control working solution to be tested can be poured after monitoring the tail current peak until more than 3 scanning curves are stable, until the inhibition of the test substance/positive control working solution to the hERG current peak reaches a stable state. Generally, the most recent three consecutive peak values of the current curve basically coincide as a criterion to judge whether the state is stable. After reaching a stable state, continue to pour one concentration of the test sample. One or more test subjects/positive controls, or multiple concentrations of the same drug, can be tested on a cell, and the different test subjects/positive controls need to be rinsed with a solvent control working fluid until the hERG current returns to more than 80% of its pre-drug level. The standard deviation of inhibition rate of all recorded cells at the same concentration was not more than 15%.

The concentration of positive control cisapride was 0.1 μM, and two cells were repeated. Cisapride inhibited hERG current by more than 50% at 0.1 μM.

TABLE 8
hERG channel inhibition activity test results

| | hERG IC₅₀ |
|---|---|
| NO. | (μM) |
| B2 | >30 |
| B22 | >30 |
| B37 | >30 |
| B41 | >30 |

According to Table 8, compounds B2, B22, B37, B41 had no inhibition on hERG channel.

### Example 23. Pharmacokinetic Evaluation

Goal 1. Evaluation of pharmacokinetic profile of candidate compounds in mice

### Experimental Procedure:

The mice pharmacokinetic characteristics of compounds were tested by standard protocols. The candidate compounds were made into clear solution for single intravenous injection (i.v.) and suspension for oral administration (p.o.). Intravenous vehicle is 5% DMSO+10% Solutol+85% Saline. Of which adjusts pH to 3 for B37. While oral vehicle is 0.5% CMCNa for B2 and B22, 5% DMSO+10% Solutol+85% Saline, PH˜3 for B37, 5% DMSO+10% Solutol+85% Saline for B41. The experiment used 6 male mice and 3 mice for intravenous at a dose of 2 mg/kg. Plasma samples were collected at 0 h (before dosing) and 0.083 h, 0.25 h, 0.5 h, 1 h, 2 h, 4 h, 8 h, 24 h after dosing. Another 3 mice were orally administrated with a dose of 10 mg/kg. Plasma samples were collected at 0 h (before dosing) and 0.25 h, 0.5 h, 1 h, 2 h, 4 h, 6 h, 8 h, 24 h after dosing.

Blood samples were placed in tubes containing K2-EDTA and stored on ice until centrifuged. The blood samples were centrifuged at 6800 g for 6 minutes at 2-8° C. within 1 h after collected, and stored frozen at approximately −80° C.

The analytical results were confirmed using quality control samples for intra-assay variation. The accuracy of >66.7% of the quality control samples and 50% of all QC samples at each concentration level were between 80-120% of the known value(s).

Standard set of parameters including area under the curve (AUC(0-t) and AUC(0-∞)), elimination half-live (T_{1/2}), maximum plasma concentration (Cmax), time to reach maximum plasma concentration (Tmax) were calculated using noncompartmental analysis modules in FDA certified pharmacokinetic program Phoenix WinNonlin 7.0 (Pharsight, USA) by the Study Director.

TABLE 9
Mice PK of test compounds

| NO. | | B2 | B22 | B37 | B41 |
|---|---|---|---|---|---|
| i.v.: 2 mg/kg | CL (mL/kg/min) | 17.2 | 25.4 | 15.5 | 12.7 |
| | Vd (L/kg) | 1.5 | 3.1 | 0.84 | 1.2 |
| | AUC(ng · h/mL) | 1935 | 1351 | 2161 | 2666 |
| | T_{1/2} (h) | 1.0 | 1.4 | 0.63 | 1.1 |
| p.o.: 10 mg/kg | Cmax (ng/mL) | 1183 | 2630 | 4253 | 6565 |
| | Tmax (h) | 3.0 | 0.33 | 0.42 | 0.5 |
| | AUC (ng · h/mL) | 4135 | 6366 | 8421 | 11859 |
| | F (%) | 43 | 94 | 78 | 89 |

Conclusion: Compounds B2, B22, B37, B41 had good plasma exposure and bioavailability in mice.

Goal 2. Evaluation of pharmacokinetic profile of candidate compounds in rats

### Experimental Procedure:

The rat pharmacokinetic characteristics of compounds were tested by standard protocols. The candidate compounds were made into clear solution for single intravenous injection (i.v.) and suspension for oral administration (p.o.). Intravenous vehicle is 5% DMSO+10% Solutol+85% Saline. Of which adjusts pH to 3 for B37. While oral vehicle is 0.5% CMCNa for B2 and B22, 5% DMSO+10% Solutol+85% Saline, pH˜3 for B37, 5% DMSO+10% Solutol+85% Saline for B41. The experiment used 6 male rats and 3 rats for intravenous at a dose of 2 mg/kg. Plasma samples were collected at 0 h (before dosing) and 0.083 h, 0.25 h, 0.5 h, 1 h, 2 h, 4 h, 8 h, 24 h after dosing. 3 rats were orally administrated at a dose of 10 mg/kg. Plasma samples were collected at 0 h (before dosing) and 0.25 h, 0.5 h, 1 h, 2 h, 4 h, 6 h, 8 h, 24 h after dosing.

Blood samples were placed in tubes containing K2-EDTA and stored on ice until centrifuged. The blood samples were centrifuged at 6800 g for 6 minutes at 2-8° C. within 1 h after collected, and stored frozen at approximately −80° C.

The analytical results were confirmed using quality control samples for intra-assay variation. The accuracy of >66.7% of the quality control samples and 50% of all QC samples at each concentration level were between 80-120% of the known value(s).

Standard set of parameters including area under the curve (AUC(0-t) and AUC(0-∞)), elimination half-live (T_{1/2}), maximum plasma concentration (Cmax), time to reach maximum plasma concentration (Tmax) were calculated using noncompartmental analysis modules in FDA certified pharmacokinetic program Phoenix WinNonlin 7.0 (Pharsight, USA) by the Study Director.

TABLE 10
Rat PK of test compounds

| NO. | | B2 | B22 | B37 | B41 |
|---|---|---|---|---|---|
| i.v.: 2 mg/kg | CL (mL/kg/min) | 13.8 | 12.7 | 10.9 | 11.6 |
| | Vd (L/kg) | 1.5 | 1.4 | 0.9 | 1.0 |
| | AUC(ng · h/mL) | 2432 | 2667 | 3094 | 2883 |
| | T_{1/2} (h) | 1.3 | 1.3 | 0.96 | 1.0 |
| p.o.: 10 mg/kg | Cmax (ng/ml) | 2025 | 694 | 1886 | 1799 |
| | Tmax (h) | 0.67 | 1.83 | 2.05 | 2.83 |
| | AUC (ng · h/mL) | 5899 | 2276 | 9419 | 6900 |
| | F (%) | 49 | 17 | 61 | 48 |

Conclusion: Compounds B2, B37, B41 had good plasma exposure and bioavailability in rat.

Goal 3. Evaluation of pharmacokinetic profile of candidate compounds in beagle dog Experimental procedure:

The beagle dog pharmacokinetic characteristics of compounds were tested by standard protocols. The candidate compounds were made into clear solution for single intravenous injection (i.v.) and suspension for oral administration (p.o.). Intravenous and oral vehicle is 5% DMSO+10% Solutol+85% Saline. The experiment used 9 dog and 3 dog for intravenous at a dose of 1 mg/kg. Plasma samples were collected at 0 h (before dosing) and 0.083 h, 0.25 h, 0.5 h, 1 h, 2 h, 4 h, 8 h, 24 h after dosing. 3 dog were orally administrated at a dose of 5 mg/kg and 3 dog were orally administrated at a dose of 15 mg/kg. Plasma samples were collected at 0 h (before dosing) and 0.25 h, 0.5 h, 1 h, 2 h, 4 h, 6 h, 8 h, 24 h after dosing.

Blood samples were placed in tubes containing K2-EDTA and stored on ice until centrifuged. The blood samples were centrifuged at 6800 g for 6 minutes at 2-8° C. within 1 h after collected, and stored frozen at approximately −80° C.

The analytical results were confirmed using quality control samples for intra-assay variation. The accuracy of >66.7% of the quality control samples and 50% of all QC samples at each concentration level were between 80-120% of the known value(s).

Standard set of parameters including area under the curve (AUC(0-t) and AUC(0-∞)), elimination half-live (T_{1/2}), maximum plasma concentration (Cmax), time to reach maximum plasma concentration (Tmax) were calculated using noncompartmental analysis modules in FDA certified pharmacokinetic program Phoenix WinNonlin 7.0 (Pharsight, USA) by the Study Director.

TABLE 11
Beagle dog PK of test compounds

| NO. | | B37 | B41 |
|---|---|---|---|
| i.v.: 1 mg/kg | CL (mL/kg/min) | 1.9 | 4.0 |
| | Vd (L/kg) | 0.33 | 0.56 |
| | AUC(ng · h/mL) | 9238 | 4897 |
| | T_{1/2} (h) | 2.2 | 2.1 |
| p.o.: 5 mg/kg | Cmax (ng/mL) | 3980 | 3099 |
| | Tmax (h) | 0.83 | 1.0 |
| | AUC(ng · h/mL) | 21314 | 19398 |
| | F (%) | 46 | 79 |
| p.o.: 15 mg/ kg | Cmax (ng/ml) | 4869 | 9871 |
| | Tmax (h) | 0.67 | 1.7 |
| | AUC(ng · h/mL) | 28609 | 88045 |
| | F (%) | 21 | 120 |

Conclusion: Compounds B37, B41 had excellent plasma exposure in beagle dog.

### Example 24. Evaluation of the Efficacy of the Compound in IL-23 Induced Psoriasis

### Experimental Procedure:

Recombinant human IL-23 (100 ng/time/mouse) was injected into the right ear of mice every two days starting from day 1 of the study. The administration group included 7 mice, with EtOH:TPGS:PEG300=5:5:95 as vehicle, compound B41 (10 mg/kg, 30 mg/kg) or BMS986165 (10 mg/kg) orally gavage twice a day the night before the first injection of IL-23. The control group was injected with vehicle alone. The experiment lasted for 14 days. The thickness of the ear was measured with a micrometer every two days.

As shown in FIG. 9, compound B41 and BMS986165 could effectively inhibit and treat IL-23 induced psoriasis. The efficacy of compound B41 was better than BMS986165 at the same dose.

1. A compound of Formula (I): 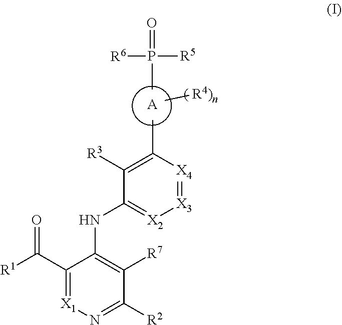
or a pharmaceutically acceptable salt, ester, solvate, prodrug, isotope-labeled derivative, or isomer thereof, wherein:
n is 0, 1, 2 or 3;
X₁ is N or CH;
each of X₂, X₃ and X₄ is independently N or CR⁸;
ring A is C₆₋₁₀ aryl or 5-10 membered heteroaryl;
R¹ is C₁₋₆ alkyl, C₁₋₆ deuterated alkyl, C₂₋₆ alkenyl, C₂₋₆ alkynyl, C₃₋₆ cycloalkyl, —NH(deuterated C₁₋₆ alkyl), or —NH(C₁₋₆ alkyl), wherein C₁₋₆ alkyl, C₁₋₆ deuterated alkyl, C₂₋₆ alkenyl, C₂₋₆ alkynyl, and C₃₋₆ cycloalkyl) are unsubstituted or substituted with one or more groups independently selected from R^{aa}; preferably, R¹ is independently C₁₋₆ alkyl, C₁₋₆ deuterated alkyl, C₂₋₆ alkenyl, C₂₋₆ alkynyl, or C₃₋₆ cycloalkyl, wherein C₁₋₆ alkyl, C₁₋₆ deuterated alkyl, C₂₋₆ alkenyl, C₂₋₆ alkynyl, or C₃₋₆ cycloalkyl, are unsubstituted or substituted with one or more groups independently selected from R^{aa};
R^{aa} is hydrogen, deuterium, halide, amino, —NO₂, —CN, —OH, or C₁₋₃ alkyl;
R² is alkyl, deuterated alkyl, halo-alkyl, alkoxy, halo-alkoxy, alkenyl, alkynyl, cycloalkyl, heterocycloalkyl, aryl, heteroaryl, —NR^{b}R^{c}, C(O)R^{a}, —C(O)NR^{b}R^{c}, —S(O)R^{a}, —S(O)₂R^{a}, —C(O)OR^{a}, —NR^{d}C(O)R^{a}, —NR^{d}C(O)NR^{b}R^{c}, —NR^{d}S(O)R^{a}, —NR^{d}SOS(O)₂R^{a}, —NR^{d}S(O)NR^{b}R^{c}, —NR^{d}S(O)₂NR^{b}R^{c}, or —NR^{d}C(O)OR₂, wherein alkyl, deuterated alkyl, halo-alkyl, alkoxy, halo-alkoxy, alkenyl, alkynyl, cycloalkyl, heterocycloalkyl, aryl, and heteroaryl are unsubstituted or substituted with one or more groups independently selected from hydrogen, deuterium, halide, amino, —NO₂, —CN, —OH, C₁₋₃ alkyl, deuterated C₁₋₃ alkyl, C₁₋₃ halo-alkyl, C₁₋₃ alkoxy, deuterated C₁₋₃ alkoxy, C₁₋₃ halo-alkoxy, C₂₋₆ alkenyl, C₂₋₆ alkynyl, substituted or unsubstituted cycloalkyl, substituted and unsubstituted heterocycloalkyl, and substituted or unsubstituted aryl;
R³ is hydrogen, halide, —OH, amino, —SH, —NO₂, —CN, C₁₋₆ alkyl, —C(O)NH₂, C₁₋₆ deuterated alkyl, —O(C₁₋₆ alkyl), —O(C₁₋₆ deuterated alkyl), C₂₋₆ alkenyl, C₂₋₆ alkynyl, C₃₋₆ cycloalkyl, C₃₋₆ heterocycloalkyl, C₆₋₁₀ aryl or 5-10 membered heteroaryl, wherein C₁₋₆ alkyl, C₁₋₆ deuterated alkyl, —O(C₁₋₆ alkyl), —O(C₁₋₆ deuterated alkyl), C₂₋₆ alkenyl, C₂₋₆ alkynyl, C₃₋₆ cycloalkyl, C₃₋₆ heterocycloalkyl C₆₋₁₀ aryl and 5-10 membered heteroaryl are unsubstituted or substituted with one or more groups independently selected from R^{aa};
if present, each R⁴ is independently hydrogen, deuterium, halide, —OH, amino, —CN, CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R^{bb} is hydrogen, deuterium, halide, amino, —NO₂, —CN, and —OH;
each of R⁵ and R⁶ is independently C₁₋₆ alkyl, C₁₋₆ deuterated alkyl, C₂₋₆ alkenyl, C₂₋₆ alkynyl, or C₃₋₆ cycloalkyl, wherein C₁₋₆ alkyl, C₁₋₆ deuterated alkyl, C₂₋₆ alkenyl, C₂₋₆ alkynyl, and C₃₋₆ cycloalkyl are unsubstituted or substituted with one or more groups selected from R^{bb}; preferably each of R⁵ and R⁶ is independently C₁₋₃ alkyl, wherein C₁₋₃ alkyl is unsubstituted or substituted with one or more groups selected from R^{bb}; or R⁵ and R⁶, together with P attached thereto, form a 5-6 membered heterocycloalkyl, wherein 5-6 membered heterocycloalkyl is unsubstituted or substituted with one or more groups selected from R^{bb};
R⁷ is hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
if present, each R⁸ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb}; and
each of R^{a}, R^{b}, R^{c} and R^{d} is independently hydrogen, deuterium, halide, amino, —NO₂, —CN, —OH, alkyl, deuterated alkyl, halo-alkyl, alkoxy, halo-alkoxy, alkenyl, alkynyl, cycloalkyl, heterocycloalkyl, aryl, or heteroaryl, wherein alkyl, deuterated alkyl, halo-alkyl, alkoxy, halo-alkoxy, alkenyl, alkynyl, cycloalkyl, heterocycloalkyl, aryl, and heteroaryl are unsubstituted or substituted with one or more groups independently selected from hydrogen, deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted cycloalkyl, and substituted and unsubstituted heterocycloalkyl, substituted or unsubstituted aryl, and substituted or unsubstituted heteroaryl; or any two of adjacent or non-adjacent R^{a}, Rh, R^{c} and R^{d} form a cycloalkyl, heterocycloalkyl, aryl or heteroaryl, wherein cycloalkyl, heterocycloalkyl, aryl and heteroaryl are unsubstituted or substituted with one or more groups selected from hydrogen, deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted 6-10 membered aryl, and substituted or unsubstituted 5-10 membered heteroaryl.

2. The compound of claim 1, or a pharmaceutically acceptable salt, ester, solvate, prodrug, isotope-labeled derivative, or isomer thereof, wherein:
R² is C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, 5-10 membered heteroaryl, —NR^{b}R^{c}, —C(O)R^{a}, —C(O)NR′RE, —S(O)R^{a}, —S(O)₂R^{a}, —C(O)OR^{a}, —NR^{d}C(O)R^{a}, —NR^{d}C(O)NR^{b}R^{c}, —NR^{d}S(O)R^{a}, —NR^{d}S(O)₂R^{a}, —NR^{d}S(O)NR^{b}R^{c}, —NR^{d}S(O)₂NR^{b}R^{c}, or —NR^{d}C(O)OR^{a}, wherein C₁₋₆ alkyl, deuterated C₁₋₆ alkyl, C₁₋₆ halo-alkyl, C₁₋₆ alkoxy, C₁₋₆ halo-alkoxy, C₂₋₆ alkynyl, C₃₋₆ cycloalkyl, 3-6 membered heterocycloalkyl, C₆₋₁₀ aryl, and 5-10 membered heteroaryl are unsubstituted or substituted with one or more groups independently selected from hydrogen, deuterium, halide, amino, —NO₂, —CN, —OH, C₁₋₃ alkyl, deuterated C₁₋₃ alkyl, C₁₋₃ halo-alkyl, C₁₋₃ alkoxy, deuterated C₁₋₃ alkoxy, C₁₋₃ halo-alkoxy, C₂₋₃ alkenyl, C₂₋₃ alkynyl, substituted or unsubstituted C₃₋₆ cycloalkyl, substituted and unsubstituted 3-6 membered heterocycloalkyl, substituted or unsubstituted C₆₋₁₀ aryl, and substituted or unsubstituted 5-10 membered heteroaryl; and
each of R^{a}, R^{b}, R^{c} and R^{d} is independently hydrogen, deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl, wherein C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, and 5-10 membered heteroaryl are unsubstituted or substituted with one or more groups independently selected from hydrogen, deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, and substituted and unsubstituted 3-6 membered heterocycloalkyl, substituted or unsubstituted C₆₋₁₀ aryl, and substituted or unsubstituted 5-10 membered heteroaryl; or any two of adjacent or non-adjacent R^{a}, R^{b}, R^{c} and R^{d} form a C₃₋₆ cycloalkyl, C₃₋₆ heterocycloalkyl, C₆₋₁₀ aryl or 5-10 membered heteroaryl, wherein C₃₋₆ cycloalkyl, C₃₋₆ heterocycloalkyl, C₆₋₁₀ aryl and 5-10 membered heteroaryl are unsubstituted or substituted with one or more groups selected from hydrogen, deuterium, halide, amino, —NO₂, —CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted C₆₋₁₀ aryl, and substituted or unsubstituted 5-10 membered heteroaryl.

3. The compound of claim 1, or a pharmaceutically acceptable salt, ester, solvate, prodrug, isotope-labeled derivative, or isomer thereof, wherein:
ring A is phenyl or 5-6 membered heteroaryl;
R¹ is C₁-3 alkyl, C₁₋₃ deuterated alkyl, C₂₋₆ alkenyl, C₂₋₆ alkynyl, C₃₋₆ cycloalkyl, —NH(deuterated C₁₋₃ alkyl), or —NH(C₁₋₆ alkyl), wherein C₁₋₃ alkyl, C₁₋₃ deuterated alkyl, C₂₋₆ alkenyl, C₂₋₆ alkynyl, and C₃₋₆ cycloalkyl are unsubstituted or substituted with one or more groups independently selected from R^{aa}; preferably, R¹ is independently C₁₋₃ alkyl, C₁₋₃ deuterated alkyl, C₂₋₆ alkenyl, C₂₋₆ alkynyl, or C₃₋₆ cycloalkyl;
R³ is-CN, C₁₋₆ alkyl, C₁₋₆ deuterated alkyl, —O(C₁₋₆ alkyl), —O(C₁₋₆ deuterated alkyl), C₃₋₆ cycloalkyl, or —C(O)NH₂;
R⁵ is C₁₋₆ alkyl or C₃₋₆ cycloalkyl; preferably C₁₋₃ alkyl or cyclopropyl; and
R⁶ is C₁₋₆ alkyl or C₃₋₆ cycloalkyl; preferably C₁₋₃ alkyl or cyclopropyl.

4. The compound of claim 1, wherein the compound is of Formula (II): 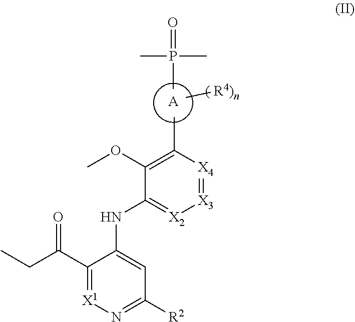
or a pharmaceutically acceptable salt, ester, solvate, prodrug, isotope-labeled derivative, or isomer thereof, wherein:
n is 0, 1, 2 or 3;
X₁ is N or CH;
each of X₂, X₃ and X₄ is independently N or CR⁸;
ring A is C₆₋₁₀ aryl or 5-10 membered heteroaryl;
R² is alkyl, deuterated alkyl, halo-alkyl, alkoxy, halo-alkoxy, alkenyl, alkynyl, cycloalkyl, heterocycloalkyl, aryl, heteroaryl, —NR^{b}R^{c}, —C(O)R^{a}, —C(O)NR^{b}R^{c}, —S(O)R^{a}, —S(O)₂R^{a}, —C(O)OR^{a}, —NR^{d}C(O)R^{a}, —NR^{d}C(O)NR^{b}R^{c}, —NR^{d}S(O)R^{a}, —NR^{d}S(O)₂R^{a}, —NR^{d}S(O)NR^{b}R^{c}, —NR^{d}S(O)₂NR^{b}R^{c}, or —NR^{d}C(O)OR^{a}, wherein alkyl, deuterated alkyl, halo-alkyl, alkoxy, halo-alkoxy, alkenyl, alkynyl, cycloalkyl, heterocycloalkyl, aryl, and heteroaryl are unsubstituted or substituted with one or more groups independently selected from hydrogen, deuterium, halide, amino, —NO₂, —CN, —OH, C₁₋₃ alkyl, deuterated C₁₋₃ alkyl, C₁₋₃ halo-alkyl, C₁₋₃ alkoxy, deuterated C₁₋₃ alkoxy, C₁₋₃ halo-alkoxy, C₂₋₆ alkenyl, C₂₋₆ alkynyl, substituted or unsubstituted cycloalkyl, substituted and unsubstituted heterocycloalkyl, and substituted or unsubstituted aryl;
if present, each R⁴ is independently hydrogen, deuterium, halide, —OH, amino, —CN, CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R^{bb} is hydrogen, deuterium, halide, amino, —NO₂, —CN, and —OH;
if present, each R⁸ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb}; and
each of R^{a}, R^{b}, R^{c} and R^{d} is independently hydrogen, deuterium, halide, amino, —NO₂, —CN, —OH, alkyl, deuterated alkyl, halo-alkyl, alkoxy, halo-alkoxy, alkenyl, alkynyl, cycloalkyl, heterocycloalkyl, aryl, or heteroaryl, wherein alkyl, deuterated alkyl, halo-alkyl, alkoxy, halo-alkoxy, alkenyl, alkynyl, cycloalkyl, heterocycloalkyl, aryl, and heteroaryl are unsubstituted or substituted with one or more groups independently selected from hydrogen, deuterium, halide, amino, —NO₂, —CN, —OH, C₁₋₃ alkyl, deuterated C₁₋₃ alkyl, C₁₋₃ halo-alkyl, C₁₋₃ alkoxy, C₁₋₃ halo-alkoxy, C₂₋₆ alkenyl, C₂₋₆ alkynyl, substituted or unsubstituted cycloalkyl, and substituted and unsubstituted heterocycloalkyl, substituted or unsubstituted aryl, and substituted or unsubstituted heteroaryl; or any two of adjacent or non-adjacent R^{a}, R^{b}, R^{c} and R^{d} form a cycloalkyl, heterocycloalkyl, aryl or heteroaryl, wherein cycloalkyl, heterocycloalkyl, aryl and heteroaryl are unsubstituted or substituted with one or more groups selected from hydrogen, deuterium, halide, amino, —NO₂, —CN, —OH, C₁₋₃ alkyl, deuterated C₁₋₃ alkyl, C₁₋₃ halo-alkyl, C₁₋₃ alkoxy, C₁₋₃ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted 6-10 membered aryl, and substituted or unsubstituted 5-10 membered heteroaryl.

5. The compound of claim 4, or a pharmaceutically acceptable salt, ester, solvate, prodrug, isotope-labeled derivative, or isomer thereof, wherein 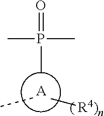
is a moiety A substituted with 0 to 3 R¹⁰,
wherein, if present, each R¹⁰ is independently hydrogen, deuterium, halide, amino, —CN, —OH, C₁₋₃ alkyl or C₁₋₃ alkoxy; and
wherein the moiety A is selected from the group consisting of:

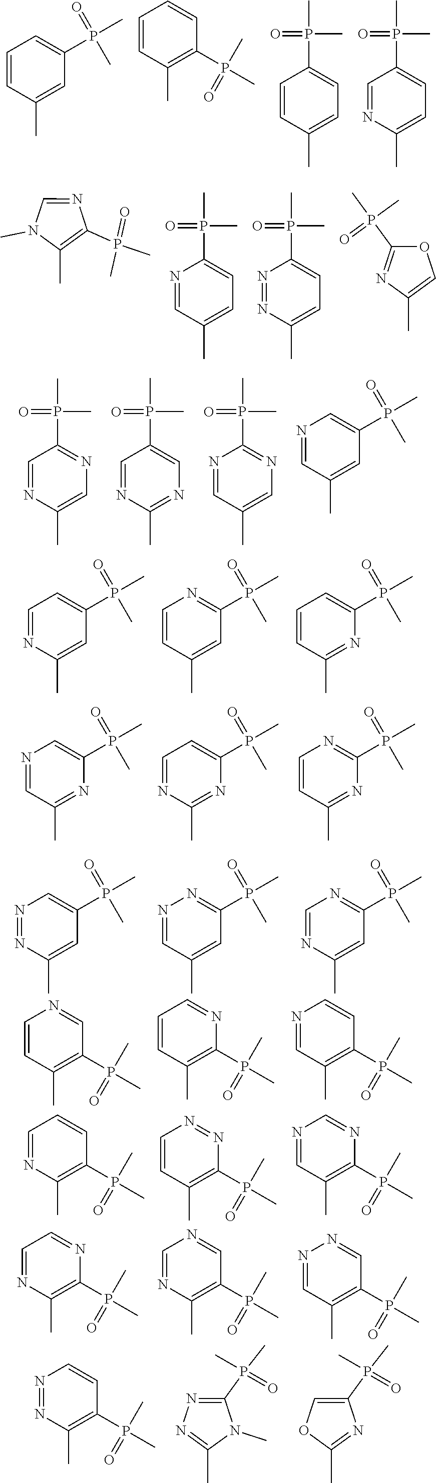

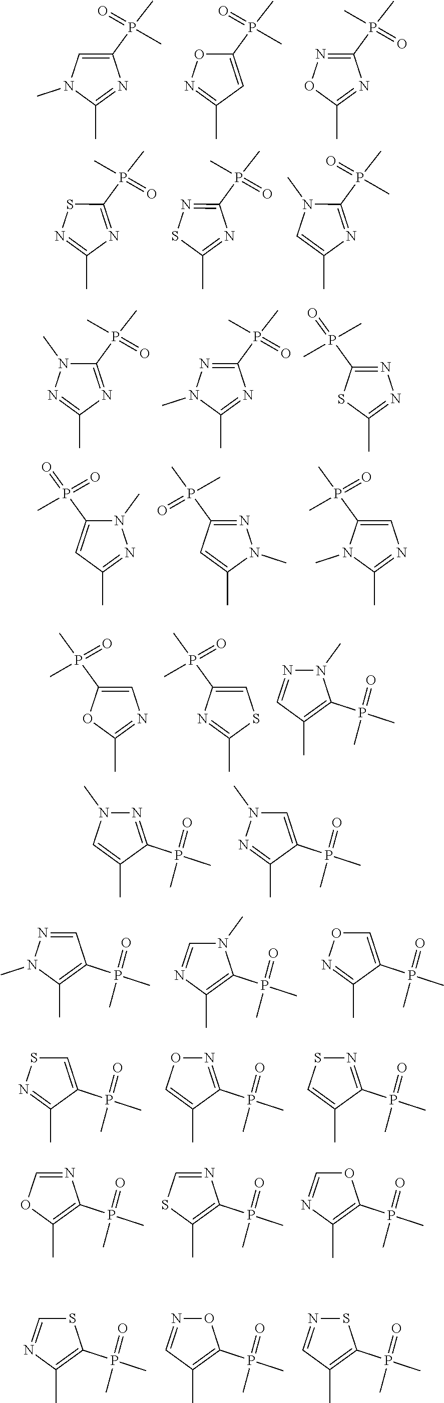

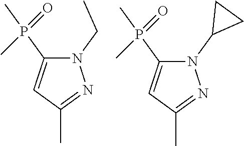

6. The compound of claim 4, or a pharmaceutically acceptable salt, ester, solvate, prodrug, isotope-labeled derivative, or isomer thereof, wherein R² is a moiety B substituted with 0 to 3 R¹¹;
wherein, if present, each R¹¹ is independently hydrogen, deuterium, halide, amino, —NO₂, —CN, —OH, C₁₋₃ alkyl or C₁₋₃ alkoxy; and
wherein the moiety B is selected from the group consisting of:

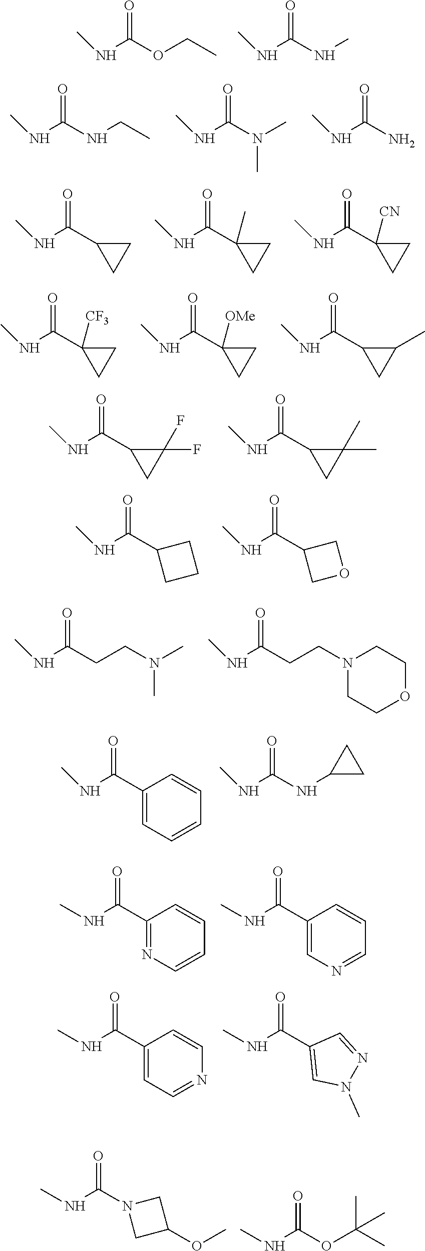

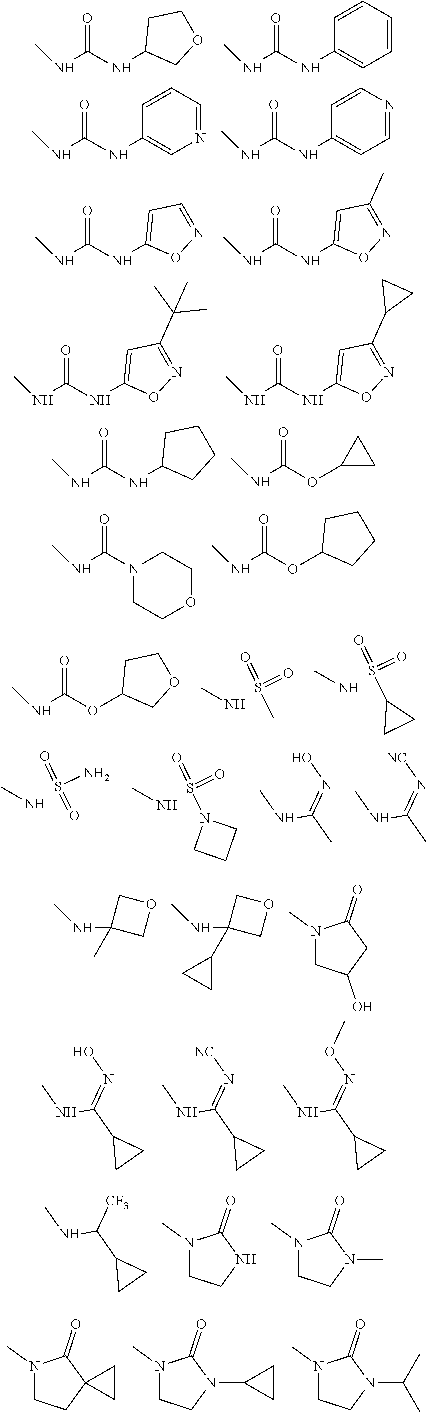

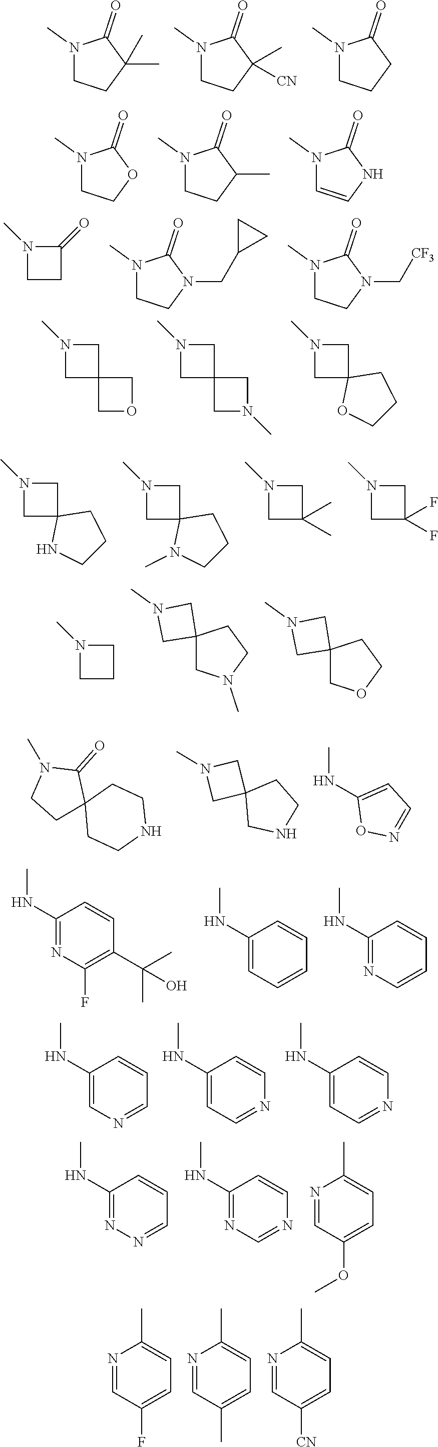

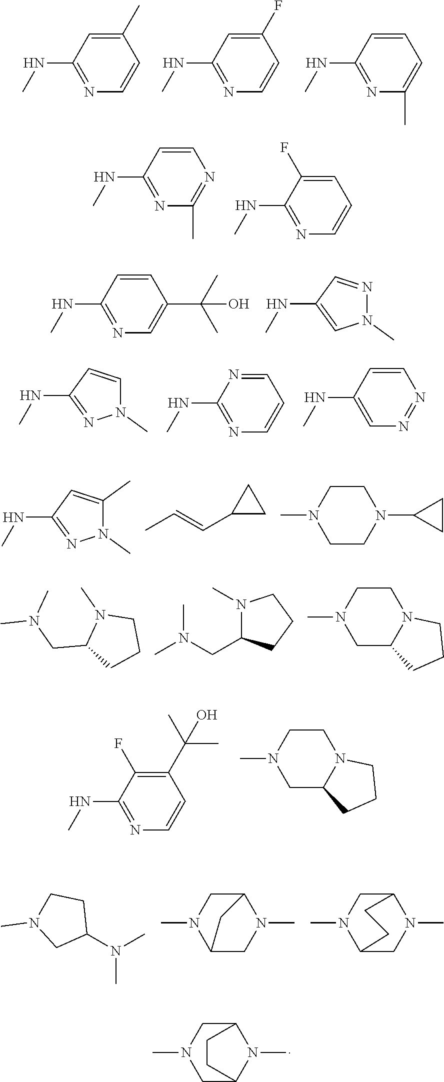

7. The compound of claim 1, wherein the compound is of Formula (III): 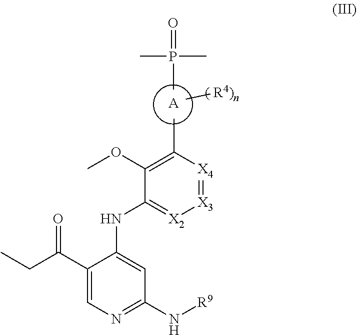
or a pharmaceutically acceptable salt, ester, solvate, prodrug, isotope-labeled derivative, or isomer thereof, wherein:
n is 0, 1, 2 or 3;
each of X₂, X₃ and X₄ is independently N or CR⁸;
ring A is C₆₋₁₀ aryl or 5-10 membered heteroaryl;
if present, each R⁴ is independently hydrogen, deuterium, halide, —OH, amino, —CN, CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R^{bb} is hydrogen, deuterium, halide, amino, —NO₂, —CN, and —OH;
if present, each R⁸ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R⁹ is C₆₋₁₀ aryl, 5-10 membered heteroaryl, —C(O)R^{a}, —NR^{d}C(O)NR^{b}R^{c}, —S(O)R^{a}, —S(O)₂R^{a}, —S(O)NR^{b}R^{c}, —S(O)₂NR^{b}R^{c}, or —C(O)OR^{a}, wherein C₆₋₁₀ aryl and 5-10 membered heteroaryl are substituted by one or more groups selected from hydrogen, deuterium, halide, amino, —NO₂, —CN, —OH, C₁₋₃ alkyl, C₁₋₃ deuterated alkyl, C₁₋₃ halo-alkyl C₁₋₃ alkoxy, C₁₋₃ halo-alkoxy, C₂₋₃ alkenyl, and C₂₋₃ alkynyl; and
each of R^{a}, R^{b}, and R^{c} is independently hydrogen, deuterium, halide, amino, —NO₂, —CN, —OH, C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl, wherein C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl are unsubstituted or substituted with one or more groups independently selected from hydrogen, deuterium, halide, amino, —NO₂, —CN, —OH, C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted.

8. The compound of claim 1, wherein the compound is of Formula (IV): 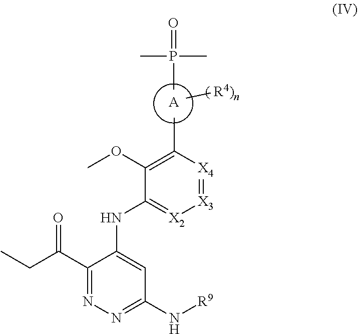
or a pharmaceutically acceptable salt, ester, solvate, prodrug, isotope-labeled derivative, or isomer thereof, wherein:
n is 0, 1, 2 or 3;
each of X₂, X₃ and X₄ is independently N or CR⁸;
ring A is C₆₋₁₀ aryl or 5-10 membered heteroaryl;
if present, each R⁴ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R^{bb} is hydrogen, deuterium, halide, amino, —NO₂, —CN, and —OH;
if present, each R⁸ is independently hydrogen, deuterium, halide, —OH, amino, —CN, CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R⁹ is C₆₋₁₀ aryl, 5-10 membered heteroaryl, —C(O)R^{a}, —C(O)NR^{b}R^{c}, —S(O)R^{a}, —S(O)₂R^{a}, —S(O)NR^{b}R^{c}, —S(O)₂NR^{b}R^{c}, or —C(O)OR^{a}, wherein C₆₋₁₀ aryl and 5-10 membered heteroaryl are substituted by one or more groups selected from hydrogen, deuterium, halide, amino, —NO₂, —CN, —OH, C₁₋₃ alkyl, C₁₋₃ deuterated alkyl, C₁₋₃ halo-alkyl C₁₋₃ alkoxy, C₁₋₃ halo-alkoxy, C₂₋₃ alkenyl, and C₂₋₃ alkynyl; and
each of R^{a}, R^{b}, and R^{c} is independently hydrogen, deuterium, halide, amino, —NO₂, —CN, —OH, C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl, wherein C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl are unsubstituted or substituted with one or more groups independently selected from hydrogen, deuterium, halide, amino, —NO₂, —CN, —OH, C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted C₆₋₁₀ aryl, and substituted or unsubstituted 5-10 membered heteroaryl.

9. The compound of claim 1, wherein the compound is of Formula (V), 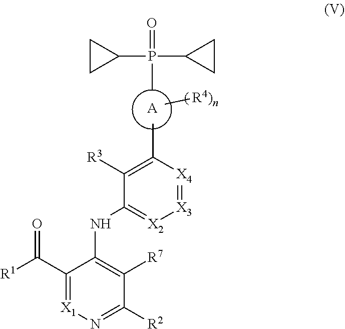
or a pharmaceutically acceptable salt, ester, solvate, prodrug, isotope-labeled derivative, or isomer thereof, wherein:
n is 0, 1, 2 or 3;
X₁ is N or CH;
each of X₂, X₃ and X₄ is independently N or CR⁸;
ring A is C₆₋₁₀ aryl or 5-10 membered heteroaryl;
R¹ is C₁₋₆ alkyl, C₁₋₆ deuterated alkyl, C₂₋₆ alkenyl, C₂₋₆ alkynyl, C₃₋₆ cycloalkyl, —NH(deuterated C₁₋₆ alkyl), or —NH(C₁₋₆ alkyl), wherein C₁₋₆ alkyl, C₁₋₆ deuterated alkyl, C₂₋₆ alkenyl, C₂₋₆ alkynyl, and C₃₋₆ cycloalkyl) are unsubstituted or substituted with one or more groups independently selected from R^{aa}; preferably, R¹ is independently C₁₋₆ alkyl, C₁₋₆ deuterated alkyl, C₂₋₆ alkenyl, C₂₋₆ alkynyl, or C₃₋₆ cycloalkyl, wherein C₁₋₆ alkyl, C₁₋₆ deuterated alkyl, C₂₋₆ alkenyl, C₂₋₆ alkynyl, or C₃₋₆ cycloalkyl, are unsubstituted or substituted with one or more groups independently selected from R^{aa};
R^{aa} is hydrogen, deuterium, halide, amino, —NO₂, —CN, —OH, or C₁₋₃ alkyl;
R² is alkyl, deuterated alkyl, halo-alkyl, alkoxy, halo-alkoxy, alkenyl, alkynyl, cycloalkyl, heterocycloalkyl, aryl, heteroaryl, —NR^{b}R^{c}, —C(O)R^{a}, —C(O)NR′RE, —S(O)R^{a}, —S(O)₂R^{a}, —C(O)OR^{a}, —NR^{d}C(O)R^{a}, —NR^{d}C(O)NR^{b}R^{c}, —NR^{d}S(O)R^{a}, —NR^{d}SOS(O)₂R^{a}, —NR^{d}S(O)NR^{b}R^{c}, —NR^{d}S(O)₂NR^{b}R^{c}, or —NR^{d}C(O)OR₂, wherein alkyl, deuterated alkyl, halo-alkyl, alkoxy, halo-alkoxy, alkenyl, alkynyl, cycloalkyl, heterocycloalkyl, aryl, and heteroaryl are unsubstituted or substituted with one or more groups independently selected from hydrogen, deuterium, halide, amino, —NO₂, CN, —OH, C₁₋₃ alkyl, deuterated C₁₋₃ alkyl, C₁₋₃ halo-alkyl, C₁₋₃ alkoxy, deuterated C₁₋₃ alkoxy, C₁₋₃ halo-alkoxy, C₂₋₆ alkenyl, C₂₋₆ alkynyl, substituted or unsubstituted cycloalkyl, substituted and unsubstituted heterocycloalkyl, and substituted or unsubstituted aryl;
R³ is hydrogen, halide, —OH, amino, —SH, —NO₂, CN, C₁₋₆ alkyl, —C(O)NH₂, C₁₋₆ deuterated alkyl, —O(C₁₋₆ alkyl), —O(C₁₋₆ deuterated alkyl), C₂₋₆ alkenyl, C₂₋₆ alkynyl, C₃₋₆ cycloalkyl, C₆₋₁₀ aryl or 5-10 membered heteroaryl, wherein C₁₋₆ alkyl, C₁₋₆ deuterated alkyl, —O(C₁₋₆ alkyl), —O(C₁₋₆ deuterated alkyl), C₂₋₆ alkenyl, C₂₋₆ alkynyl, C₃₋₆ cycloalkyl, C₆₋₁₀ aryl and 5-10 membered heteroaryl are unsubstituted or substituted with one or more groups independently selected from R^{aa};
if present, each R⁴ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R^{bb} is hydrogen, deuterium, halide, amino, —NO₂, —CN, and —OH;
R⁷ is hydrogen, deuterium, halide, —OH, amino, CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein
C₁₋₆ alkyl, C₃₋₆ cycloalkyl, C₂₋₆ alkenyl and C₂₋₆ alkynyl are unsubstituted or substituted with one or more groups independently selected from R^{bb};
if present, each R⁸ is independently hydrogen, deuterium, halide, —OH, amino, —CN, CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb}; and
each of R^{a}, R^{b}, R^{c} and R^{d} is independently hydrogen, deuterium, halide, amino, —NO₂, —CN, —OH, alkyl, deuterated alkyl, halo-alkyl, alkoxy, halo-alkoxy, alkenyl, alkynyl, cycloalkyl, heterocycloalkyl, aryl, or heteroaryl, wherein alkyl, deuterated alkyl, halo-alkyl, alkoxy, halo-alkoxy, alkenyl, alkynyl, cycloalkyl, heterocycloalkyl, aryl, and heteroaryl are unsubstituted or substituted with one or more groups independently selected from hydrogen, deuterium, halide, amino, —NO₂, CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted cycloalkyl, and substituted and unsubstituted heterocycloalkyl, substituted or unsubstituted aryl, and substituted or unsubstituted heteroaryl; or any two of adjacent or non-adjacent R^{a}, R^{b}, R^{c} and R^{d} form a cycloalkyl, heterocycloalkyl, aryl or heteroaryl, wherein cycloalkyl, heterocycloalkyl, aryl and heteroaryl are unsubstituted or substituted with one or more groups selected from hydrogen, deuterium, halide, amino, —NO₂, CN, —OH, C₁₋₆ alkyl, deuterated C₁₋₆ alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted 6-10 membered aryl, and substituted or unsubstituted 5-10 membered heteroaryl.

10. The compound of claim 9, or a pharmaceutically acceptable salt, ester, solvate, prodrug, isotope-labeled derivative, or isomer thereof, wherein 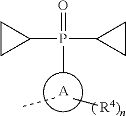
is a moiety C substituted with 0 to 3 R¹⁰,
wherein, if present, each R¹⁰ is independently hydrogen, deuterium, halide, amino, —CN, —OH, C₁₋₃ alkyl or C₁₋₃ alkoxy; and
wherein the moiety C is selected from the group consisting of:

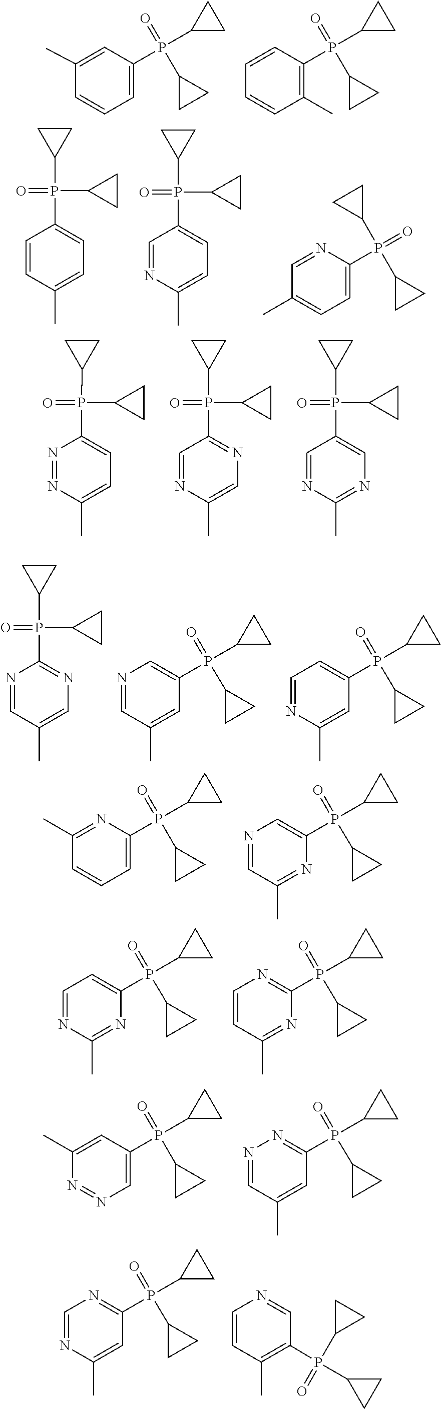

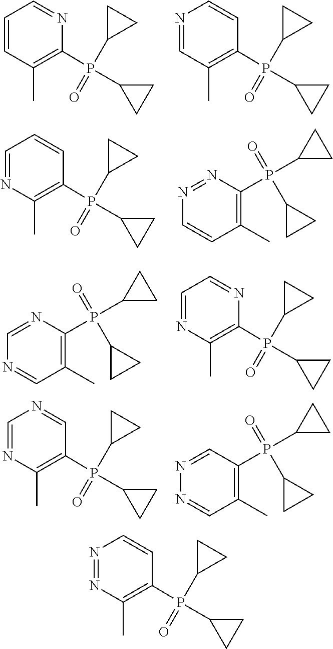

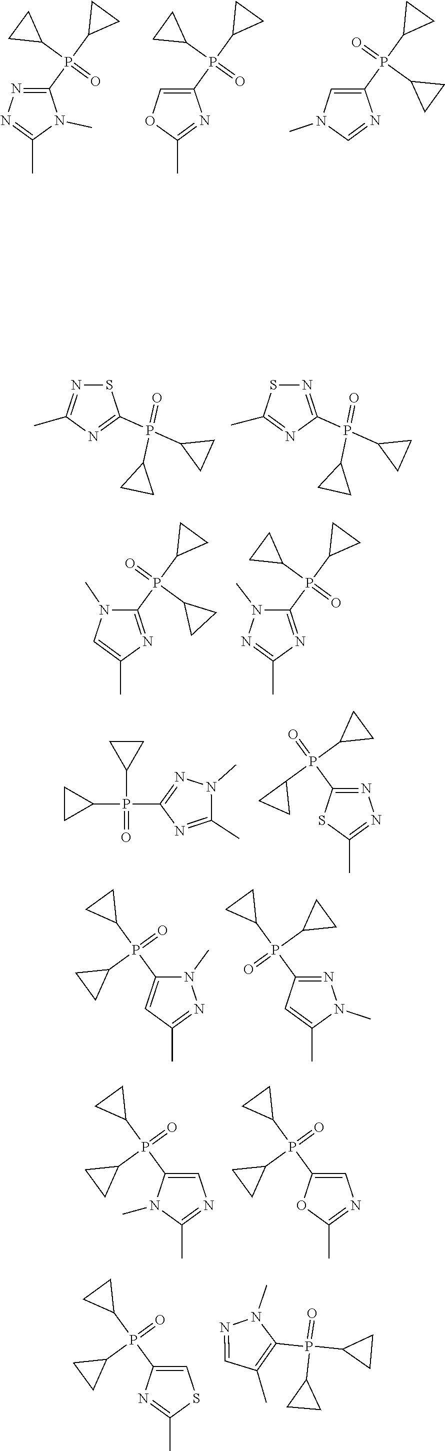

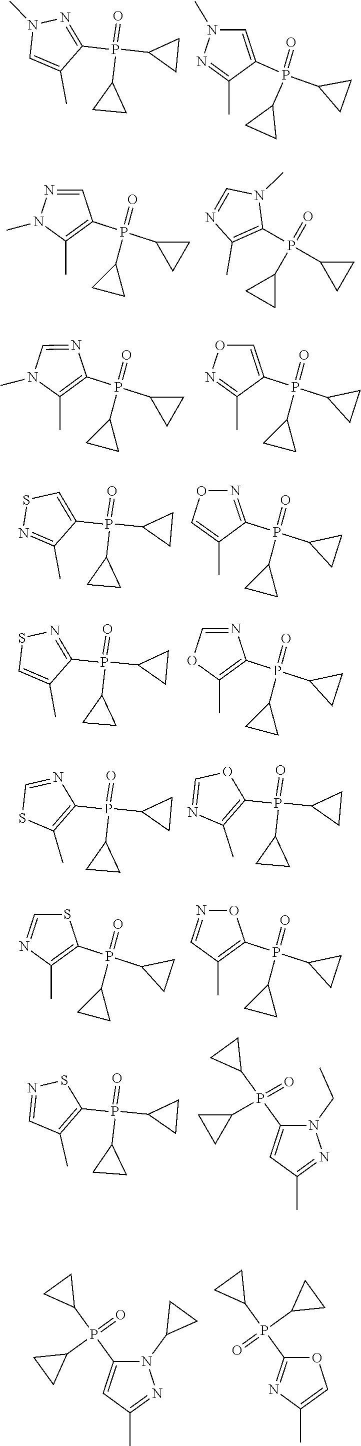

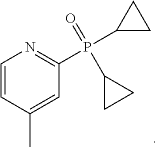

11. The compound of claim 9, or a pharmaceutically acceptable salt, ester, solvate, prodrug, isotope-labeled derivative, or isomer thereof, wherein R² is a moiety D substituted with 0 to 3 R¹¹;
wherein, if present, each R¹¹ is independently hydrogen, deuterium, halide, amino, —NO₂, —CN, —OH, C₁₋₃ alkyl or C₁₋₃ alkoxy; and
wherein the moiety D is selected from the group consisting of:

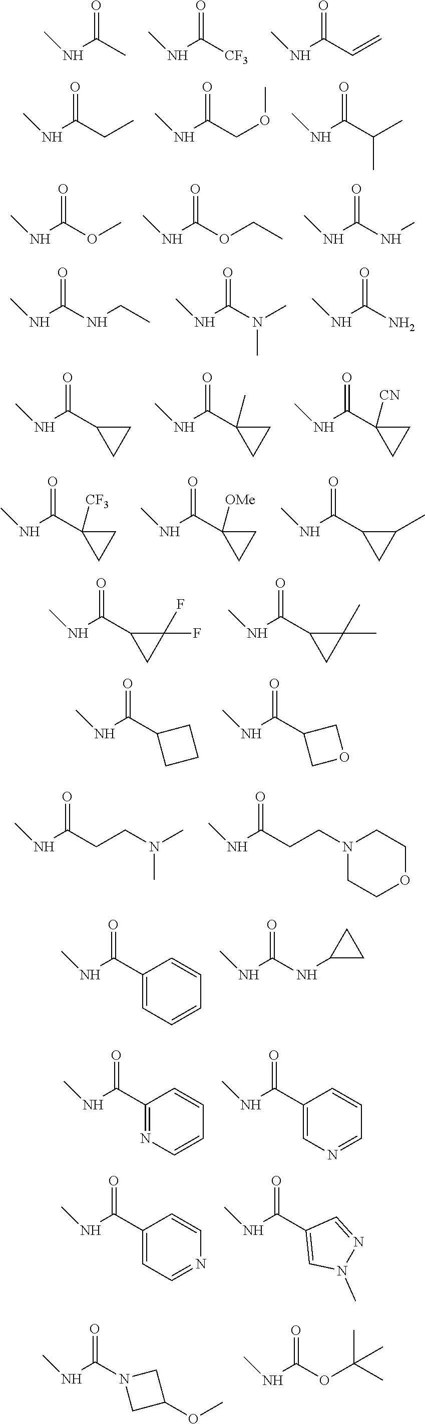

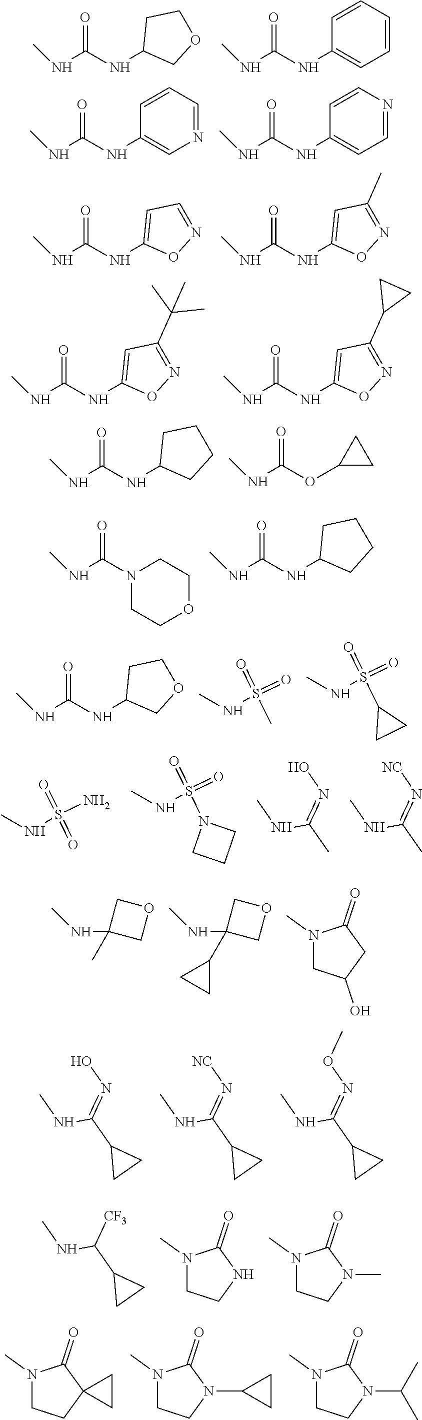

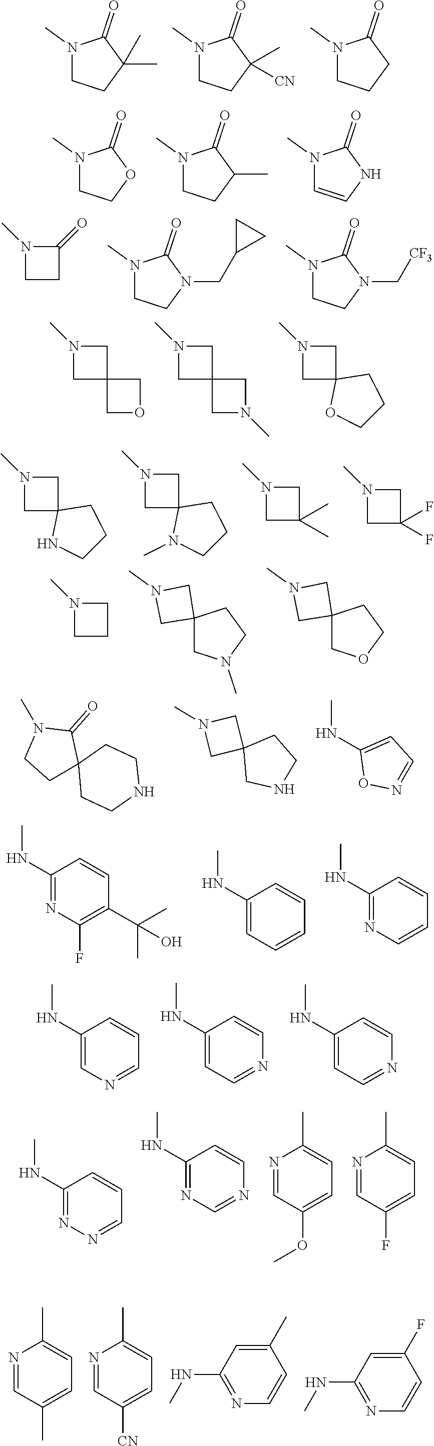

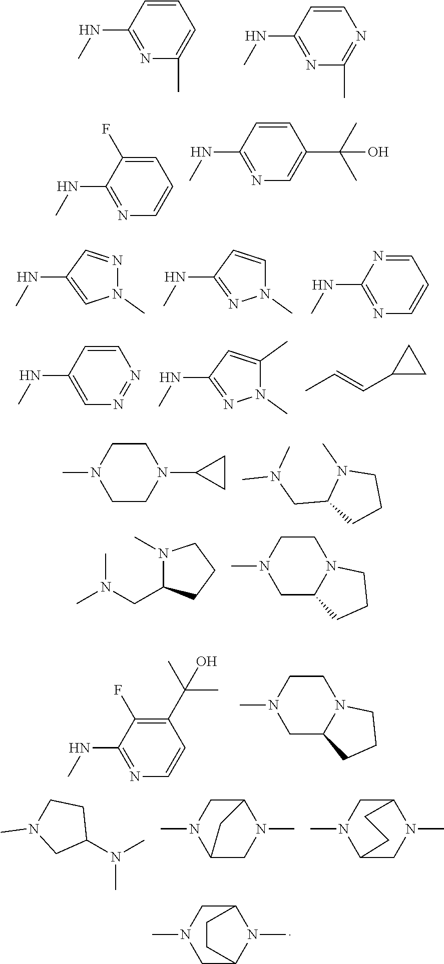

12. The compound of claim 1, wherein the compound is of Formula (VI): 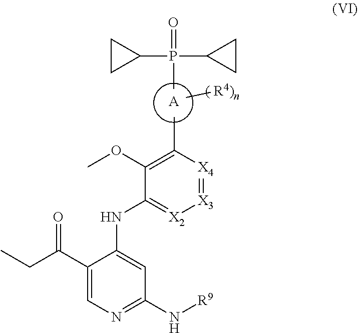
or a pharmaceutically acceptable salt, ester, solvate, prodrug, isotope-labeled derivative, or isomer thereof, wherein:
n is 0, 1, 2 or 3;
each of X₂, X₃ and X₄ is independently N or CR⁸;
ring A is C₆₋₁₀ aryl or 5-10 membered heteroaryl;
if present, each R⁴ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R^{bb} is hydrogen, deuterium, halide, amino, —NO₂, —CN, and —OH;
if present, each R⁸ is independently hydrogen, deuterium, halide, —OH, amino, —CN, CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R⁹ is C₆₋₁₀ aryl, 5-10 membered heteroaryl, —C(O)R^{a}, —C(O)NR^{b}R^{c}, —S(O)R^{a}, —S(O)₂R^{a}, —S(O)NR^{b}R^{c}, —S(O)₂NR^{b}R^{c}, or —C(O)OR^{a}, wherein C₆₋₁₀ aryl and 5-10 membered heteroaryl are substituted by one or more groups selected from hydrogen, deuterium, halide, amino, —NO₂, —CN, —OH, C₁₋₃ alkyl, C₁₋₃ deuterated alkyl, C₁₋₃ halo-alkyl C₁₋₃ alkoxy, C₁₋₃ halo-alkoxy, C₂₋₃ alkenyl, and C₂₋₃ alkynyl; and
each of R^{a}, R^{b}, and R^{c} is independently hydrogen, deuterium, halide, amino, —NO₂, —CN, —OH, C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl, wherein C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl are unsubstituted or substituted with one or more groups independently selected from hydrogen, deuterium, halide, amino, —NO₂, —CN, —OH, C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted C₆₋₁₀ aryl, and substituted or unsubstituted 5-10 membered heteroaryl.

13. The compound of claim 1, wherein the compound is of Formula (VII): 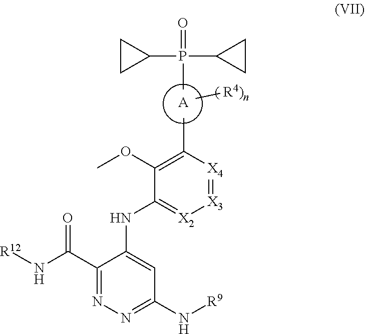
or a pharmaceutically acceptable salt, ester, solvate, prodrug, isotope-labeled derivative, or isomer thereof, wherein:
n is 0, 1, 2 or 3;
each of X₂, X₃ and X₄ is independently N or CR⁸;
ring A is C₆₋₁₀ aryl or 5-10 membered heteroaryl;
if present, each R⁴ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R^{bb} is hydrogen, deuterium, halide, amino, —NO₂, —CN, and —OH;
if present, each R⁸ is independently hydrogen, deuterium, halide, —OH, amino, —CN, —CF₃, C₁₋₆ alkyl, C₃₋₆ cycloalkyl, —O(C₁₋₆ alkyl), —NH(C₁₋₆ alkyl), —N(C₁₋₆ alkyl)₂, C₂₋₆ alkenyl or C₂₋₆ alkynyl, wherein C₁₋₆ alkyl, C₃₋₆ cycloalkyl, C₂₋₆ alkenyl and C₂₋₆ alkynyl are unsubstituted or substituted with one or more groups independently selected from R^{bb};
R⁹ is C₆₋₁₀ aryl, 5-10 membered heteroaryl, —C(O)R^{a}, —NR^{d}C(O)NR^{b}R^{c}, —S(O)R^{a}, —S(O)₂R^{a}, —S(O)NR^{b}R^{c}, —S(O)₂NR^{b}R^{c}, or —C(O)OR^{a}, wherein C₆₋₁₀ aryl and 5-10 membered heteroaryl are substituted by one or more groups selected from hydrogen, deuterium, halide, amino, —NO₂, —CN, —OH, C₁₋₃ alkyl, C₁₋₃ deuterated alkyl, C₁₋₃ halo-alkyl C₁₋₃ alkoxy, C₁₋₃ halo-alkoxy, C₂₋₃ alkenyl, and C₂₋₃ alkynyl;
R¹² is methyl, CH₂D, —CHD₂, or —CD₃; and
each of R^{a}, R^{b}, and R^{c} is independently hydrogen, deuterium, halide, amino, —NO₂, —CN, —OH, C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl, wherein C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, C₃₋₆ cycloalkyl, 3-6 membered heterocycloalkyl, C₆₋₁₀ aryl, or 5-10 membered heteroaryl are unsubstituted or substituted with one or more groups independently selected from hydrogen, deuterium, halide, amino, —NO₂, —CN, —OH, C₁₋₆ alkyl, C₁₋₆ deuterated alkyl, C₁₋₆ halo-alkyl, C₁₋₆ alkoxy, C₁₋₆ halo-alkoxy, C₂₋₆ alkenyl, C₂₋₆ alkynyl, substituted or unsubstituted C₃₋₆ cycloalkyl, substituted and unsubstituted 3-6 membered heterocycloalkyl, substituted or unsubstituted C₆₋₁₀ aryl, and substituted or unsubstituted 5-10 membered heteroaryl.

14. A compound or a pharmaceutically acceptable salt, ester, solvate, prodrug, isotope-labeled derivative, or isomer thereof, wherein the compound is selected from the group consisting of:

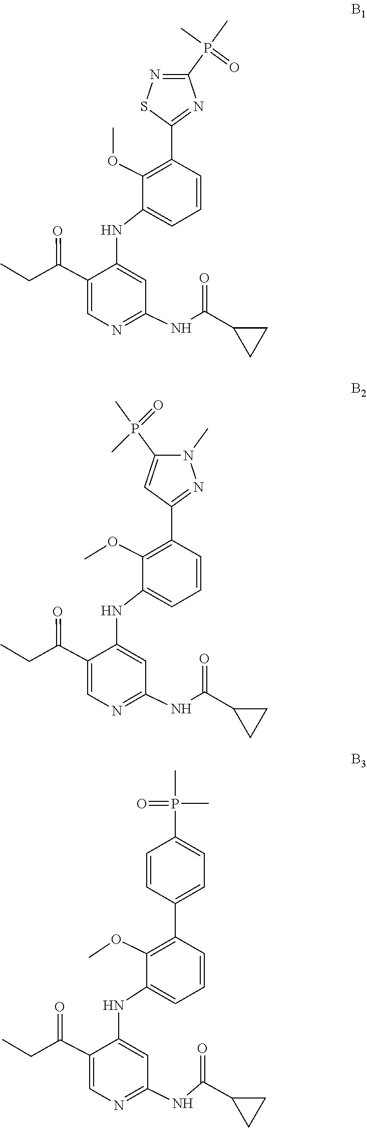 [SMILES: CCC(=O)c1cnc(NC(=O)C2CC2)cc1Nc1cccc(-c2nc(P(C)(C)=O)ns2)c1OC ; CCC(=O)c1cnc(NC(=O)C2CC2)cc1Nc1cccc(-c2cc(P(C)(C)=O)n(C)n2)c1OC ; CCC(=O)c1cnc(NC(=O)C2CC2)cc1Nc1cccc(-c2ccc(P(C)(C)=O)cc2)c1OC]

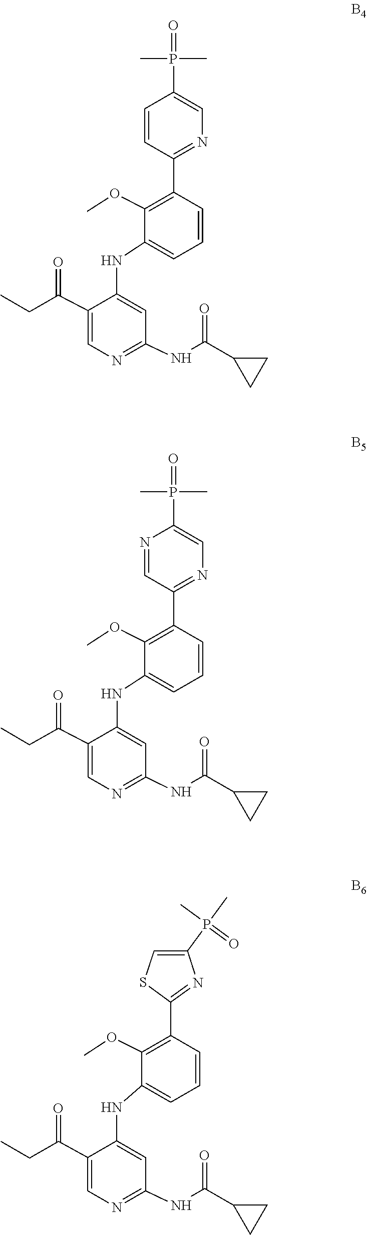 [SMILES: CCC(=O)c1cnc(NC(=O)C2CC2)cc1Nc1cccc(-c2ccc(P(C)(C)=O)cn2)c1OC ; CCC(=O)c1cnc(NC(=O)C2CC2)cc1Nc1cccc(-c2cnc(P(C)(C)=O)cn2)c1OC ; CCC(=O)c1cnc(NC(=O)C2CC2)cc1Nc1cccc(-c2nc(P(C)(C)=O)cs2)c1OC]

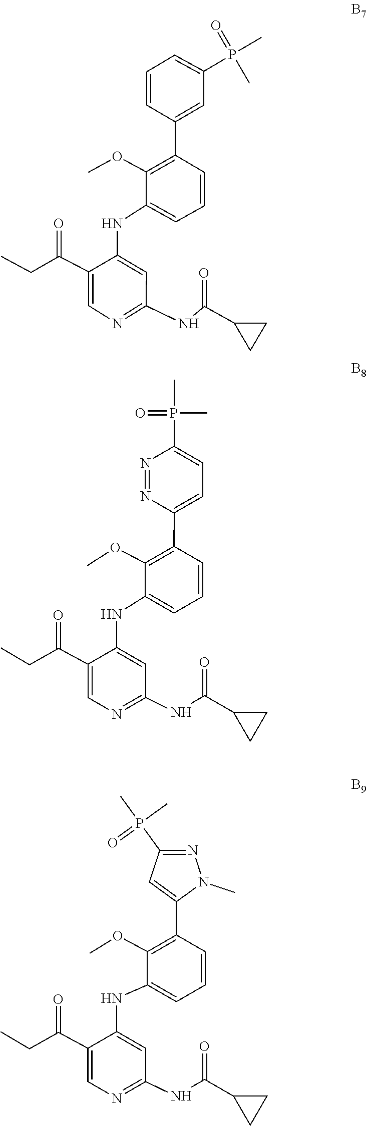 [SMILES: CCC(=O)c1cnc(NC(=O)C2CC2)cc1Nc1cccc(-c2cccc(P(C)(C)=O)c2)c1OC ; CCC(=O)c1cnc(NC(=O)C2CC2)cc1Nc1cccc(-c2ccc(P(C)(C)=O)nn2)c1OC ; CCC(=O)c1cnc(NC(=O)C2CC2)cc1Nc1cccc(-c2cc(P(C)(C)=O)nn2C)c1OC]

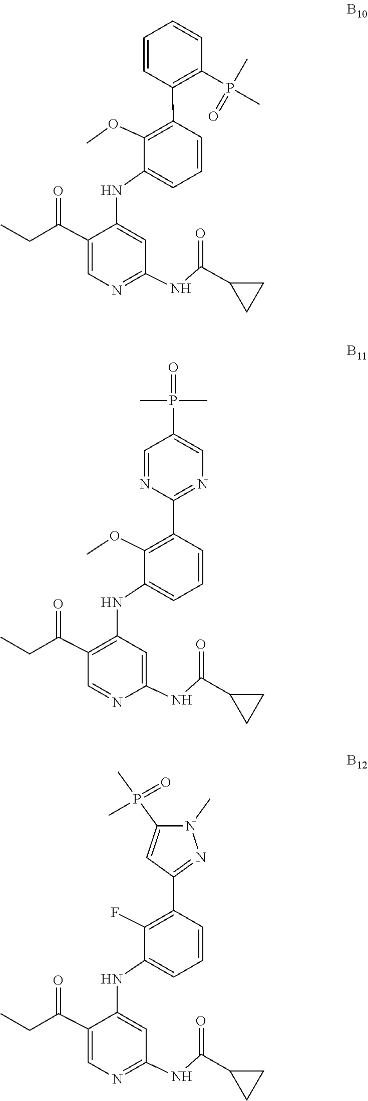 [SMILES: CCC(=O)c1cnc(NC(=O)C2CC2)cc1Nc1cccc(-c2ccccc2P(C)(C)=O)c1OC ; CCC(=O)c1cnc(NC(=O)C2CC2)cc1Nc1cccc(-c2ncc(P(C)(C)=O)cn2)c1OC ; CCC(=O)c1cnc(NC(=O)C2CC2)cc1Nc1cccc(-c2cc(P(C)(C)=O)n(C)n2)c1F]

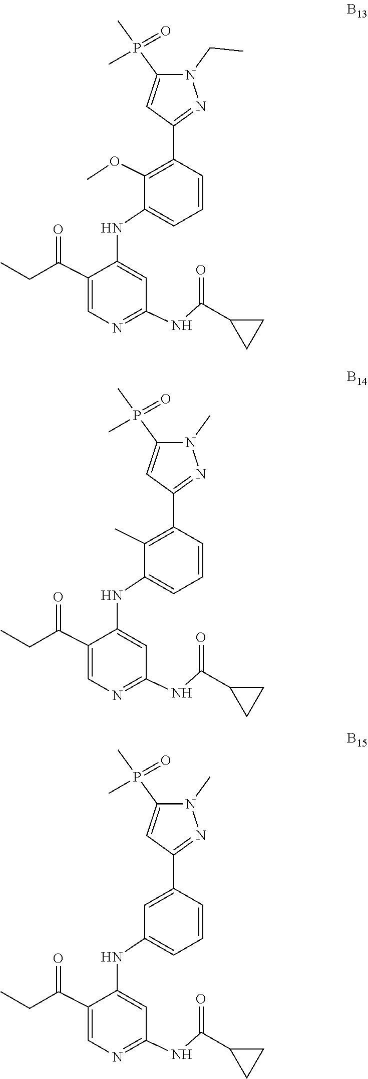 [SMILES: CCC(=O)c1cnc(NC(=O)C2CC2)cc1Nc1cccc(-c2cc(P(C)(C)=O)n(CC)n2)c1OC ; CCC(=O)c1cnc(NC(=O)C2CC2)cc1Nc1cccc(-c2cc(P(C)(C)=O)n(C)n2)c1C ; CCC(=O)c1cnc(NC(=O)C2CC2)cc1Nc1cccc(-c2cc(P(C)(C)=O)n(C)n2)c1]

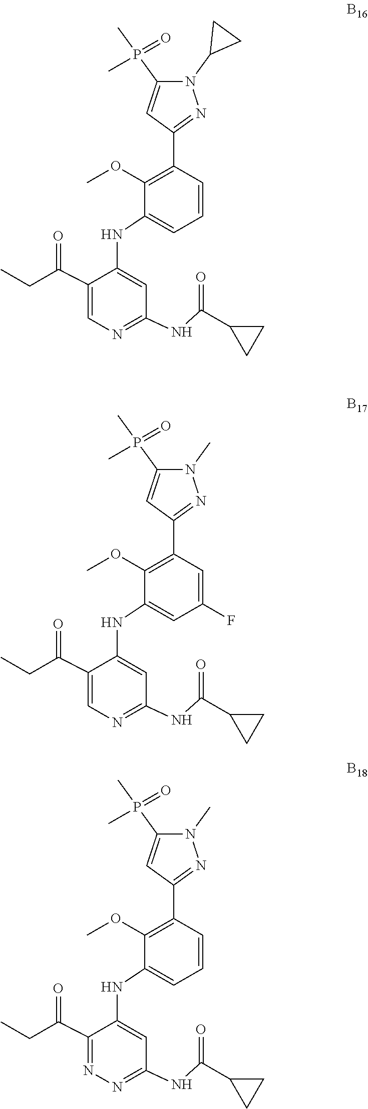 [SMILES: CCC(=O)c1cnc(NC(=O)C2CC2)cc1Nc1cccc(-c2cc(P(C)(C)=O)n(C3CC3)n2)c1OC ; CCC(=O)c1cnc(NC(=O)C2CC2)cc1Nc1cc(F)cc(-c2cc(P(C)(C)=O)n(C)n2)c1OC ; CCC(=O)c1nnc(NC(=O)C2CC2)cc1Nc1cccc(-c2cc(P(C)(C)=O)n(C)n2)c1OC]

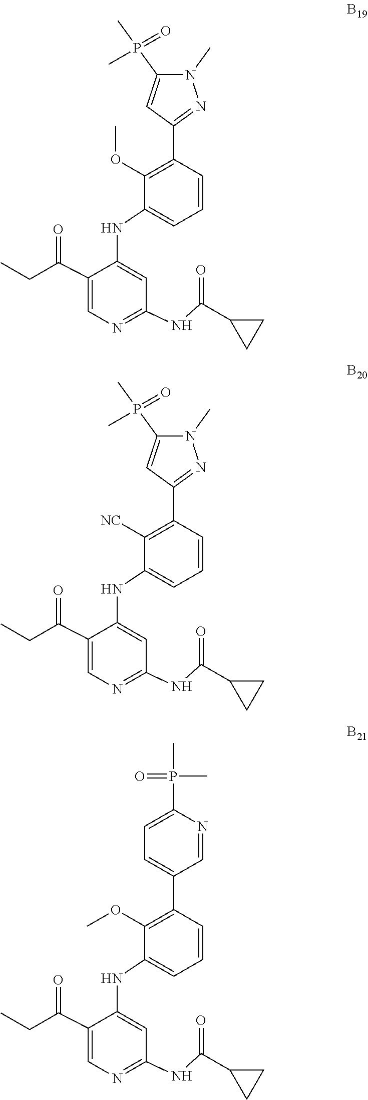 [SMILES: CCC(=O)c1cnc(NC(=O)C2CC2)cc1Nc1cccc(-c2cc(P(C)(C)=O)n(C)n2)c1OC ; CCC(=O)c1cnc(NC(=O)C2CC2)cc1Nc1cccc(-c2cc(P(C)(C)=O)n(C)n2)c1C#N ; CCC(=O)c1cnc(NC(=O)C2CC2)cc1Nc1cccc(-c2ccc(P(C)(C)=O)nc2)c1OC]

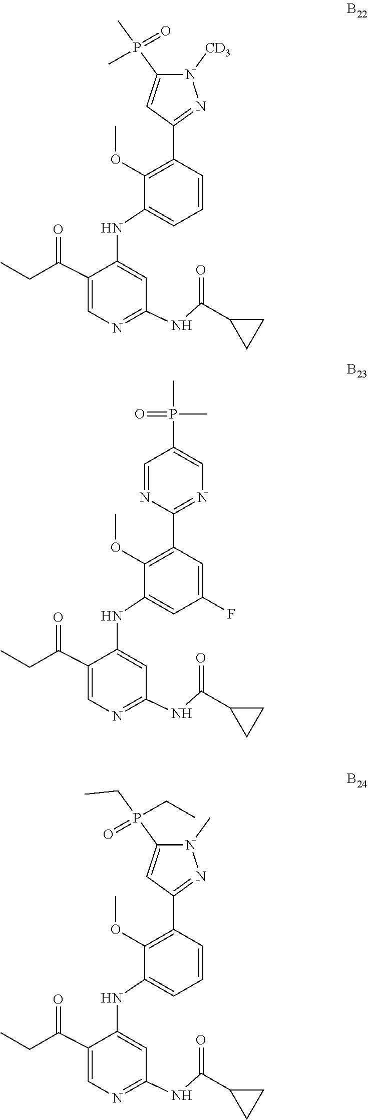 [SMILES: [2H]C([2H])([2H])n1nc(-c2cccc(Nc3cc(NC(=O)C4CC4)ncc3C(=O)CC)c2OC)cc1P(C)(C)=O ; CCC(=O)c1cnc(NC(=O)C2CC2)cc1Nc1cc(F)cc(-c2ncc(P(C)(C)=O)cn2)c1OC ; CCC(=O)c1cnc(NC(=O)C2CC2)cc1Nc1cccc(-c2cc(P(=O)(CC)CC)n(C)n2)c1OC]

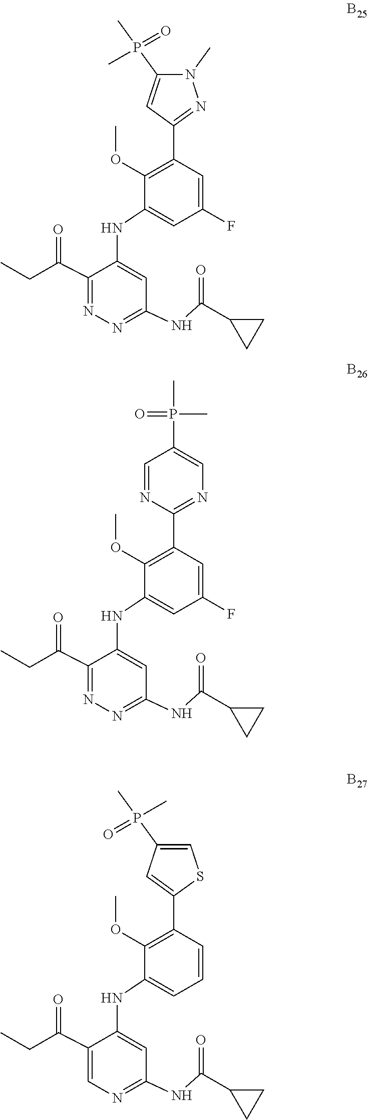 [SMILES: CCC(=O)c1nnc(NC(=O)C2CC2)cc1Nc1cc(F)cc(-c2cc(P(C)(C)=O)n(C)n2)c1OC ; CCC(=O)c1nnc(NC(=O)C2CC2)cc1Nc1cc(F)cc(-c2ncc(P(C)(C)=O)cn2)c1OC ; CCC(=O)c1cnc(NC(=O)C2CC2)cc1Nc1cccc(-c2cc(P(C)(C)=O)cs2)c1OC]

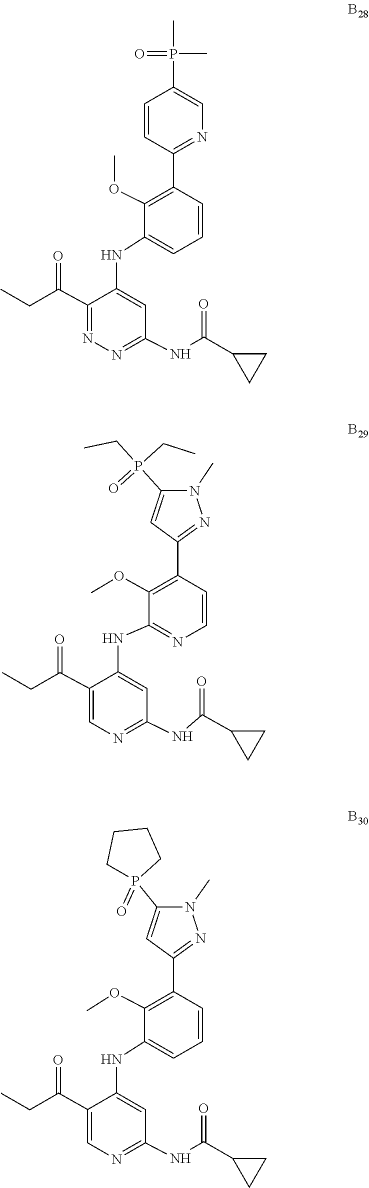 [SMILES: CCC(=O)c1nnc(NC(=O)C2CC2)cc1Nc1cccc(-c2ccc(P(C)(C)=O)cn2)c1OC ; CCC(=O)c1cnc(NC(=O)C2CC2)cc1Nc1nccc(-c2cc(P(=O)(CC)CC)n(C)n2)c1OC ; CCC(=O)c1cnc(NC(=O)C2CC2)cc1Nc1cccc(-c2cc(P3(=O)CCCC3)n(C)n2)c1OC]

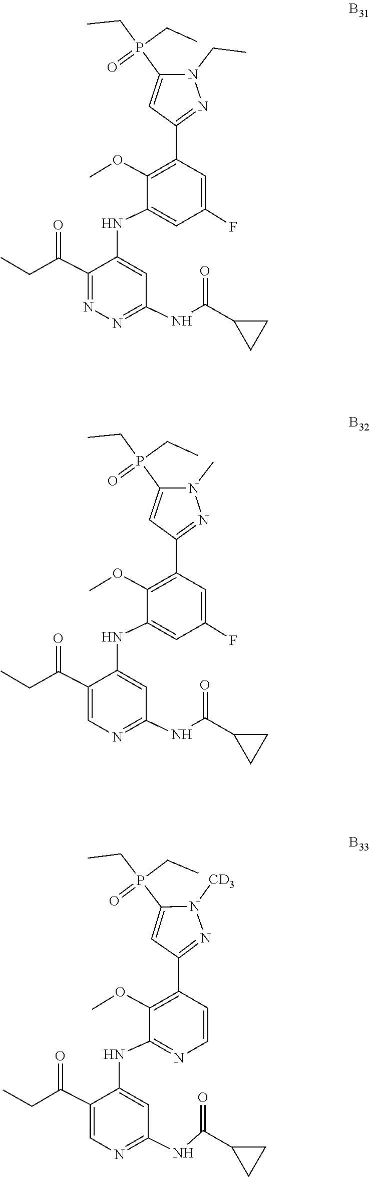 [SMILES: CCC(=O)c1nnc(NC(=O)C2CC2)cc1Nc1cc(F)cc(-c2cc(P(=O)(CC)CC)n(CC)n2)c1OC ; CCC(=O)c1cnc(NC(=O)C2CC2)cc1Nc1cc(F)cc(-c2cc(P(=O)(CC)CC)n(C)n2)c1OC ; [2H]C([2H])([2H])n1nc(-c2ccnc(Nc3cc(NC(=O)C4CC4)ncc3C(=O)CC)c2OC)cc1P(=O)(CC)CC]

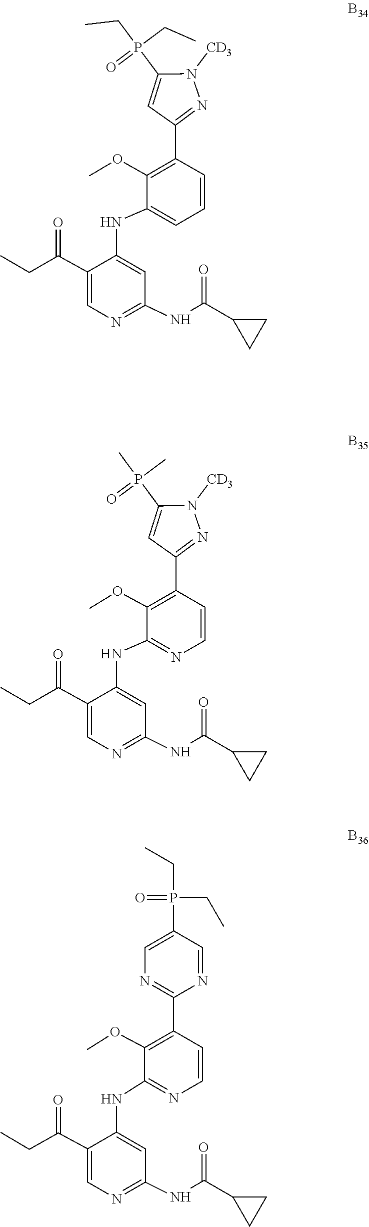 [SMILES: [2H]C([2H])([2H])n1nc(-c2cccc(Nc3cc(NC(=O)C4CC4)ncc3C(=O)CC)c2OC)cc1P(=O)(CC)CC ; [2H]C([2H])([2H])n1nc(-c2ccnc(Nc3cc(NC(=O)C4CC4)ncc3C(=O)CC)c2OC)cc1P(C)(C)=O ; CCC(=O)c1cnc(NC(=O)C2CC2)cc1Nc1nccc(-c2ncc(P(=O)(CC)CC)cn2)c1OC]

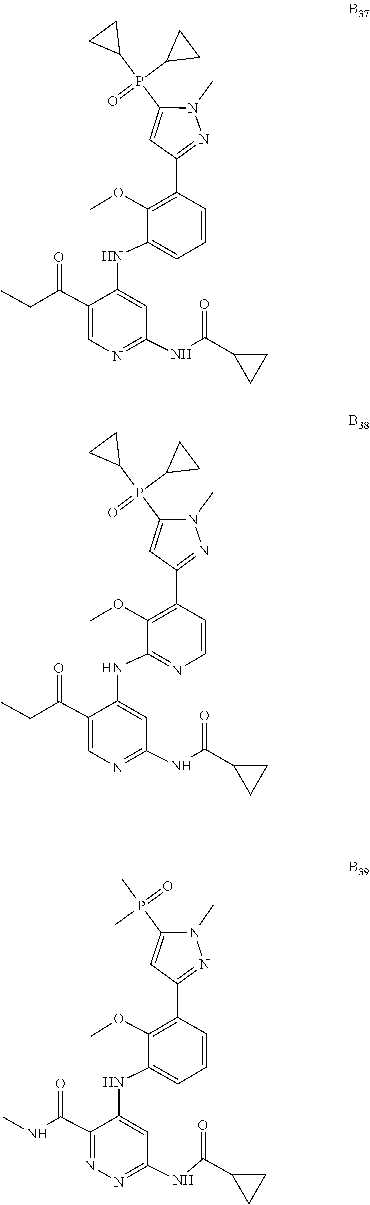 [SMILES: CCC(=O)c1cnc(NC(=O)C2CC2)cc1Nc1cccc(-c2cc(P(=O)(C3CC3)C3CC3)n(C)n2)c1OC ; CCC(=O)c1cnc(NC(=O)C2CC2)cc1Nc1nccc(-c2cc(P(=O)(C3CC3)C3CC3)n(C)n2)c1OC ; CNC(=O)c1nnc(NC(=O)C2CC2)cc1Nc1cccc(-c2cc(P(C)(C)=O)n(C)n2)c1OC]

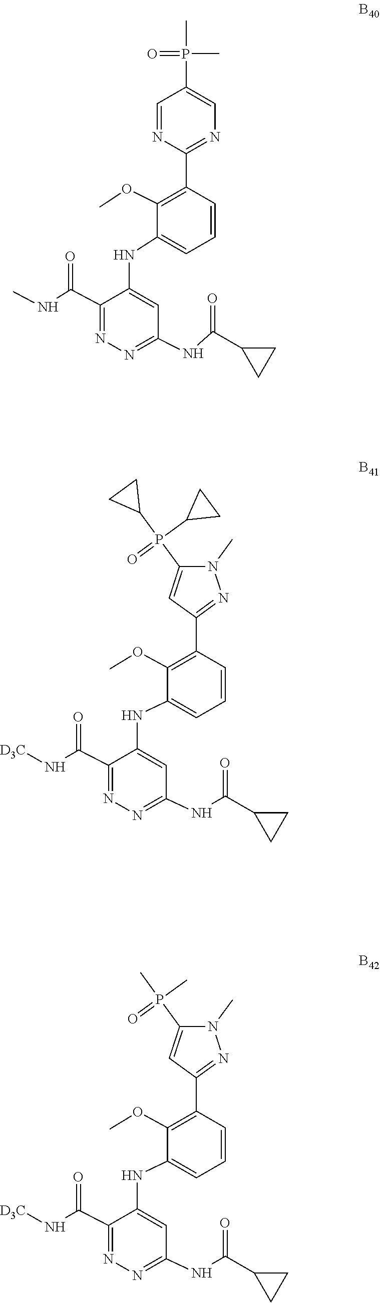 [SMILES: CNC(=O)c1nnc(NC(=O)C2CC2)cc1Nc1cccc(-c2ncc(P(C)(C)=O)cn2)c1OC ; [2H]C([2H])([2H])NC(=O)c1nnc(NC(=O)C2CC2)cc1Nc1cccc(-c2cc(P(=O)(C3CC3)C3CC3)n(C)n2)c1OC ; [2H]C([2H])([2H])NC(=O)c1nnc(NC(=O)C2CC2)cc1Nc1cccc(-c2cc(P(C)(C)=O)n(C)n2)c1OC]

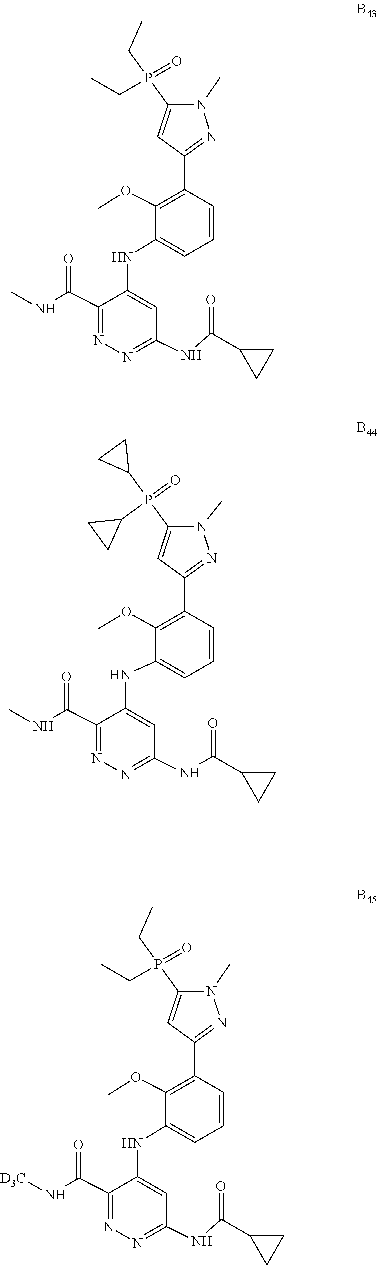 [SMILES: CCP(=O)(CC)c1cc(-c2cccc(Nc3cc(NC(=O)C4CC4)nnc3C(=O)NC)c2OC)nn1C ; CNC(=O)c1nnc(NC(=O)C2CC2)cc1Nc1cccc(-c2cc(P(=O)(C3CC3)C3CC3)n(C)n2)c1OC ; [2H]C([2H])([2H])NC(=O)c1nnc(NC(=O)C2CC2)cc1Nc1cccc(-c2cc(P(=O)(CC)CC)n(C)n2)c1OC]

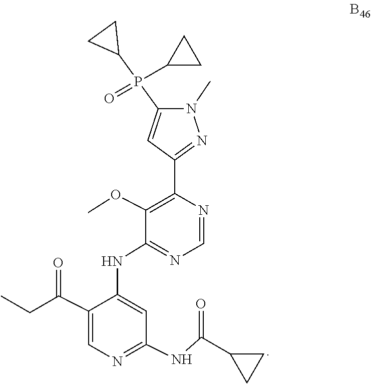 [SMILES: CCC(=O)c1cnc(NC(=O)C2CC2)cc1Nc1ncnc(-c2cc(P(=O)(C3CC3)C3CC3)n(C)n2)c1OC]

15. A pharmaceutical composition comprising a therapeutically effective amount of the compound of claim 1 or a pharmaceutically acceptable salt, ester, solvate, prodrug, isotope-labeled derivative, or isomer thereof, and a pharmaceutically acceptable carrier.

16. A composition comprising:
(i) the compound of claim 1; and
(ii) one or more additional therapeutic agent selected from the group consisting of anti-autoimmune/anti-inflammatory agent, anti-tumor/anti-cancer agent, anti-allergic agent, anti-transplant rejection agent, anti-neurodegenerative agent, anti-asthma agent and other anti-obstructive airway disease agent.

17. A method for treating a disease or disorder by inhibiting TYK2 mediated signal transduction in a subject suffering therefrom, comprising administering to the subject a therapeutically effective amount of the compound of claim 1, or a pharmaceutically acceptable salt, ester, solvate, prodrug, isotope-labeled derivative, or isomer thereof, wherein the disease or disorder is autoimmune disease or inflammation disease, cancer or tumor, allergy, transplant rejection, neurodegenerative disease, asthma or other obstructive airway diseases;
wherein the autoimmune disease or inflammation disease is enteritis, skin disease, eye disease, arthritis, Hashimoto's thyroiditis, autoimmune hemolytic anemia, autoimmune atrophic gastritis, autoimmune encephalomyelitis, Goodpasture syndrome, autoimmune thrombocytopenia, sympathetic ophthalmitis, myositis, primary biliary cirrhosis, hepatitis, primary sclerosing cholangitis, chronic invasive hepatitis, nonalcoholic fatty liver disease, nonalcoholic steatohepatitis, ulcerative colitis, membranous glomerulopathy, systemic lupus erythematosus, rheumatoid arthritis, psoriatic arthritis, polyarthritis dermatomyositis, type I Interferonopathies (including Aicardi-Goutières syndrome) and other systemic sclerosis caused by overexpression of type I interferon, Mendelian disease, multiple arteritis nodosa, multiple sclerosis, relapsing multiple sclerosis, primary progressive multiple sclerosis, secondary progressive multiple sclerosis and bullous pemphigus, Cogan's syndrome, ankylosing spondylitis, Wegener's granulomatosis, autoimmune alopecia, diabetes, or thyroid inflammation;
wherein the enteritis is Crohn's disease, ulcerative colitis, inflammatory bowel disease, celiac disease, proctitis, cosinophilic gastroenteritis, or mastocytosis;
wherein the skin disease is atopic dermatitis, eczema, psoriasis, scleroderma, pruritus or other symptoms of itching, vitiligo, or alopecia;
wherein the eye disease is keratoconjunctivitis, uveitis (including uveitis associated with Behçet's disease and uveitis caused by the lens), keratitis, herpetic keratitis, keratoconus, muscular dystrophic epithelial keratitis inflammation, corneal leukopenia, anterior uveitis, scleritis, Mooren's ulcer, Graves ophthalmopathy, Vogt-Koyanagi-Harada syndrome, keratoconjunctivitis sicca, vesicular, iridocyclitis iridosarcoidosis, endocrine ophthalmopathy, sympathetic ophthalmitis, allergic conjunctivitis, or ocular neovascularization;
wherein the diabetes is type 1 diabetes or diabetic complications;
wherein the cancer or tumor is digestive/gastrointestinal cancers, colon cancers, liver cancers, skin cancers (including mast cell and squamous cell carcinomas), breast cancers, ovarian cancers, leukemia), kidney cancer, lung cancer, muscle cancer, bone cancer, bladder cancer, brain cancer, melanoma (including oral and metastatic melanoma), Kaposi's sarcoma (including multiple myeloma), myeloproliferative disorders, proliferative diabetic retinopathy, or diseases/tumors associated with vascular hyperplasia;
wherein the neurodegenerative disease is motor neuron disease, Alzheimer's disease, Parkinson's disease, amyotrophic lateral sclerosis, Huntington's disease, cerebral ischemia, neurodegenerative diseases caused by trauma, injury, glutamate neurotoxicity or hypoxia, stroke, myocardial ischemia, renal ischemia, heart disease, cardiac hypertrophy, atherosclerosis, arteriosclerosis, ischemia/reperfusion injury of organ hypoxia or platelet aggregation;
wherein the allergy is allergic dermatitis in subjects (including allergic diseases in horses, such as allergy to bites), summer eczema, itchy horseshoes, cramps, airway inflammation, recurrent airway obstruction, airway hyperresponsiveness, and chronic obstructive pulmonary disease;
wherein the asthma or other obstructive airway diseases are chronic or excessive asthma, delayed asthma, bronchitis, bronchial asthma, allergic asthma, intrinsic asthma, extrinsic asthma or dusty asthma; and
wherein the transplant rejection is islet transplant rejection, bone marrow transplant rejection, graft-versus-host disease, organ and cell transplant rejection (the organ and cell are bone marrow, cartilage, cornea, heart, intervertebral disc, islet, kidney, extremity, liver, lung, muscle, myoblasts, nerves, pancreas, skin, small intestine or trachea), or xenograft rejection.
wherein the autoimmune disease or inflammation disease is enteritis, skin disease, eye disease, arthritis, Hashimoto's thyroiditis, autoimmune hemolytic anemia, autoimmune atrophic gastritis, autoimmune encephalomyelitis, Goodpasture syndrome, autoimmune thrombocytopenia, sympathetic ophthalmitis, myositis, primary biliary cirrhosis, hepatitis, primary sclerosing cholangitis, chronic invasive hepatitis, nonalcoholic fatty liver disease, nonalcoholic steatohepatitis, ulcerative colitis, membranous glomerulopathy, systemic lupus erythematosus, rheumatoid arthritis, psoriatic arthritis, polyarthritis dermatomyositis, type I Interferonopathies (including Aicardi-Goutières syndrome) and other systemic sclerosis caused by overexpression of type I interferon, Mendelian disease, multiple arteritis nodosa, multiple sclerosis, relapsing multiple sclerosis, primary progressive multiple sclerosis, secondary progressive multiple sclerosis and bullous pemphigus, Cogan's syndrome, ankylosing spondylitis, Wegener's granulomatosis, autoimmune alopecia, diabetes, or thyroid inflammation;
wherein the enteritis is Crohn's disease, ulcerative colitis, inflammatory bowel disease, celiac disease, proctitis, cosinophilic gastroenteritis, or mastocytosis;
wherein the skin disease is atopic dermatitis, eczema, psoriasis, scleroderma, pruritus or other symptoms of itching, vitiligo, or alopecia;
wherein the eye disease is keratoconjunctivitis, uveitis (including uveitis associated with Behçet's disease and uveitis caused by the lens), keratitis, herpetic keratitis, keratoconus, muscular dystrophic epithelial keratitis inflammation, corneal leukopenia, anterior uveitis, scleritis, Mooren's ulcer, Graves ophthalmopathy, Vogt-Koyanagi-Harada syndrome, keratoconjunctivitis sicca, vesicular, iridocyclitis iridosarcoidosis, endocrine ophthalmopathy, sympathetic ophthalmitis, allergic conjunctivitis, or ocular neovascularization;
wherein the diabetes is type 1 diabetes or diabetic complications;
wherein the cancer or tumor is digestive/gastrointestinal cancers, colon cancers, liver cancers, skin cancers (including mast cell and squamous cell carcinomas), breast cancers, ovarian cancers, prostate cancers, lymphomas, leukemia (including acute myeloid leukemia and chronic myeloid leukemia), kidney cancer, lung cancer, muscle cancer, bone cancer, bladder cancer, brain cancer, melanoma (including oral and metastatic melanoma), Kaposi's sarcoma (including multiple myeloma), myeloproliferative disorders, proliferative diabetic retinopathy, or diseases/tumors associated with vascular hyperplasia;
wherein the neurodegenerative disease is motor neuron disease, Alzheimer's disease, Parkinson's disease, amyotrophic lateral sclerosis, Huntington's disease, cerebral ischemia, neurodegenerative diseases caused by trauma, injury, glutamate neurotoxicity or hypoxia, stroke, myocardial ischemia, renal ischemia, heart disease, cardiac hypertrophy, atherosclerosis, arteriosclerosis, ischemia/reperfusion injury of organ hypoxia or platelet aggregation;
wherein the allergy is allergic dermatitis in mammals (including allergic diseases in horses, such as allergy to bites), summer eczema, itchy horseshoes, cramps, airway inflammation, recurrent airway obstruction, airway hyperresponsiveness, and chronic obstructive pulmonary disease;
wherein the asthma or other obstructive airway diseases are chronic or excessive asthma, delayed asthma, bronchitis, bronchial asthma, allergic asthma, intrinsic asthma, extrinsic asthma or dusty asthma; and
wherein the transplant rejection is islet transplant rejection, bone marrow transplant rejection, graft-versus-host disease, organ and cell transplant rejection (the organ and cell are bone marrow, cartilage, cornea, heart, intervertebral disc, islet, kidney, extremity, liver, lung, muscle, myoblasts, nerves, pancreas, skin, small intestine or trachea), or xenograft rejection.

18. The compound of claim 1, or a pharmaceutically acceptable salt, ester, solvate, prodrug, isotope-labeled derivative, or isomer thereof, for use in the treatment of a disease or disorder by inhibiting TYK2 mediated signal transduction in a subject suffering therefrom,
wherein the disease or disorder is autoimmune disease or inflammation disease, cancer or tumor, allergy, transplant rejection, neurodegenerative disease, asthma or other obstructive airway diseases;
wherein the autoimmune disease or inflammation disease is enteritis, skin disease, eye disease, arthritis, Hashimoto's thyroiditis, autoimmune hemolytic anemia, autoimmune atrophic gastritis, autoimmune encephalomyelitis, Goodpasture syndrome, autoimmune thrombocytopenia, sympathetic ophthalmitis, myositis, primary biliary cirrhosis, hepatitis, primary sclerosing cholangitis, chronic invasive hepatitis, nonalcoholic fatty liver disease, nonalcoholic steatohepatitis, ulcerative colitis, membranous glomerulopathy, systemic lupus erythematosus, rheumatoid arthritis, psoriatic arthritis, polyarthritis dermatomyositis, type I Interferonopathies (including Aicardi-Goutières syndrome) and other systemic sclerosis caused by overexpression of type I interferon, Mendelian disease, multiple arteritis nodosa, multiple sclerosis, relapsing multiple sclerosis, primary progressive multiple sclerosis, secondary progressive multiple sclerosis and bullous pemphigus, Cogan's syndrome, ankylosing spondylitis, Wegener's granulomatosis, autoimmune alopecia, diabetes, or thyroid inflammation;
wherein the enteritis is Crohn's disease, ulcerative colitis, inflammatory bowel disease, celiac disease, proctitis, cosinophilic gastroenteritis, or mastocytosis;
wherein the skin disease is atopic dermatitis, eczema, psoriasis, scleroderma, pruritus or other symptoms of itching, vitiligo, or alopecia;
wherein the eye disease is keratoconjunctivitis, uveitis (including uveitis associated with Behçet's disease and uveitis caused by the lens), keratitis, herpetic keratitis, keratoconus, muscular dystrophic epithelial keratitis inflammation, corneal leukopenia, anterior uveitis, scleritis, Mooren's ulcer, Graves ophthalmopathy, Vogt-Koyanagi-Harada syndrome, keratoconjunctivitis sicca, vesicular, iridocyclitis iridosarcoidosis, endocrine ophthalmopathy, sympathetic ophthalmitis, allergic conjunctivitis, or ocular neovascularization;
wherein the diabetes is type 1 diabetes or diabetic complications;
wherein the cancer or tumor is digestive/gastrointestinal cancers, colon cancers, liver cancers, skin cancers (including mast cell and squamous cell carcinomas), breast cancers, ovarian cancers, leukemia), kidney cancer, lung cancer, muscle cancer, bone cancer, bladder cancer, brain cancer, melanoma (including oral and metastatic melanoma), Kaposi's sarcoma (including multiple myeloma), myeloproliferative disorders, proliferative diabetic retinopathy, or diseases/tumors associated with vascular hyperplasia;
wherein the neurodegenerative disease is motor neuron disease, Alzheimer's disease, Parkinson's disease, amyotrophic lateral sclerosis, Huntington's disease, cerebral ischemia, neurodegenerative diseases caused by trauma, injury, glutamate neurotoxicity or hypoxia, stroke, myocardial ischemia, renal ischemia, heart disease, cardiac hypertrophy, atherosclerosis, arteriosclerosis, ischemia/reperfusion injury of organ hypoxia or platelet aggregation;
wherein the allergy is allergic dermatitis in subjects (including allergic diseases in horses, such as allergy to bites), summer eczema, itchy horseshoes, cramps, airway inflammation, recurrent airway obstruction, airway hyperresponsiveness, and chronic obstructive pulmonary disease;
wherein the asthma or other obstructive airway diseases are chronic or excessive asthma, delayed asthma, bronchitis, bronchial asthma, allergic asthma, intrinsic asthma, extrinsic asthma or dusty asthma; and
wherein the transplant rejection is islet transplant rejection, bone marrow transplant rejection, graft-versus-host disease, organ and cell transplant rejection (the organ and cell are bone marrow, cartilage, cornea, heart, intervertebral disc, islet, kidney, extremity, liver, lung, muscle, myoblasts, nerves, pancreas, skin, small intestine or trachea), or xenograft rejection.
